(19) United States
(12) Patent Application Publication
(10) Pub. No.: US 2025/0059297 A1
(43) Pub. Date: Feb. 20, 2025

(54) ANTI-HER2/TROP2 ANTIBODIES AND USES THEREOF

(71) Applicant: Biocytogen Pharmaceuticals (Beijing) Co., Ltd., Beijing (CN)

(72) Inventors: Chengzhang Shang, Beijing (CN); Liu Yang, Beijing (CN); Yanfei Han, Beijing (CN); Yuelei Shen, Beijing (CN)

(21) Appl. No.: 18/724,021

(22) PCT Filed: Jan. 19, 2023
(86) PCT No.: PCT/CN2023/073039

§ 371 Date: Jun. 25, 2024

(30) Foreign Application Priority Data
Jan. 26, 2022 (WO) PCT/CN2022/074078
Aug. 4, 2022 (WO) PCT/CN2022/110153
Nov. 1, 2022 (WO) PCT/CN2022/128951

(51) Int. Cl. C07K 16/32; A61K 39/00; A61P 35/00; C07K 16/30

(52) U.S. Cl. CPC C07K 16/32; A61P 35/00; C07K 16/30; A61K 2039/505; C07K 2317/14; C07K 2317/24; C07K 2317/31; C07K 2317/33; C07K 2317/565; C07K 2317/622

(57) ABSTRACT

Provided are anti-HER2 antibodies or antigen binding fragments thereof, anti-TROP2 antibodies or antigen binding fragments thereof, antigen-binding protein constructs (e.g., bispecific antibodies or antigen-binding fragments thereof) that specifically bind to two different antigens (e.g., HER2 and TROP2), and antibody drug conjugates.

### CLAIM OF PRIORITY

This application claims the benefit of PCT Application No. PCT/CN2022/074078, filed on Jan. 26, 2022, PCT Application No. PCT/CN2022/110153, filed on Aug. 4, 2022, and PCT Application No. PCT/CN2022/128951, filed on Nov. 1, 2022. The entire contents of the foregoing are incorporated herein by reference.

### TECHNICAL FIELD

This disclosure relates to antibodies or antigen-binding fragments thereof, antigen-binding protein constructs (e.g., bispecific antibodies), and antibody drug conjugates.

### BACKGROUND

Cancer is currently one of the diseases that have the highest human mortality. According to the World Health Organization statistical data, in 2012, the number of global cancer incidence and death cases reached 14 million and 8.2 million, respectively. In China, the newly diagnosed cancer cases are 3.07 million, and the death toll is 2.2 million.

Recent clinical and commercial success of anticancer antibodies has created great interest in antibody-based therapeutics. There is a need to further develop antibodies and antibody-drug conjugates for treating cancers.

### SUMMARY

This disclosure relates to anti-HER2 antibodies or antigen binding fragments thereof, anti-TROP2 antibodies or antigen binding fragments thereof, antigen-binding protein constructs (e.g., bispecific antibodies or antigen-binding fragments thereof) that specifically bind to two different antigens (e.g., HER2 and TROP2), and antibody drug conjugates involving these antibodies or antigen binding fragments thereof.

In one aspect, the disclosure is related to an antibody or antigen-binding fragment thereof that binds to HER2 (Human epidermal growth factor receptor 2) comprising:
a heavy chain variable region (VH) comprising complementarity determining regions (CDRs) 1, 2, and 3, wherein the VH CDR1 region comprises an amino acid sequence that is at least 80%, 85%, 90%, 95% or 100% identical to a selected VH CDR1 amino acid sequence, the VH CDR2 region comprises an amino acid sequence that is at least 80%, 85%, 90%, 95% or 100% identical to a selected VH CDR2 amino acid sequence, and the VH CDR3 region comprises an amino acid sequence that is at least 80%, 85%, 90%, 95% or 100% identical to a selected VH CDR3 amino acid sequence; and
a light chain variable region (VL) comprising CDRs 1, 2, and 3, wherein the VL CDR1 region comprises an amino acid sequence that is at least 80%, 85%, 90%, 95% or 100% identical to a selected VL CDR1 amino acid sequence, the VL CDR2 region comprises an amino acid sequence that is at least 80%, 85%, 90%, 95% or 100% identical to a selected VL CDR2 amino acid sequence, and the VL CDR3 region comprises an amino acid sequence that is at least 80%, 85%, 90%, 95% or 100% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 4-6, respectively;
(7) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 4-6, respectively;
(8) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 4-6, respectively;
(9) the selected VH CDRs 1, 2, 3 amino acid sequences are set forth in SEQ ID NOs: 31-33, respectively, and the selected VL CDRs 1, 2, 3 amino acid sequences are set forth in SEQ ID NOs: 4-6, respectively;
(10) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 4-6, respectively.

In some embodiments, the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 1-3, respectively, according to the Kabat numbering scheme.

In some embodiments, the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 1-3, respectively, according to the Kabat numbering scheme.

In some embodiments, the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 1-3, respectively, according to the Kabat numbering scheme.

In some embodiments, the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 1-3, respectively, according to the Kabat numbering scheme.

In some embodiments, the VH comprises CDRs 1, 2, 3 with the amino acid sequences set forth in SEQ ID NOs: 19-21 respectively, and the VL comprises CDRs 1, 2, 3 with the amino acid sequences set forth in SEQ ID NOs: 1-3, respectively, according to the Kabat numbering scheme.

In some embodiments, the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 4-6, respectively, according to the Chothia numbering scheme.

In some embodiments, the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 4-6, respectively, according to the Chothia numbering scheme.

In some embodiments, the VH comprises CDRs 1, 2, 3 with the amino acid sequences set forth in SEQ ID NOs: 28-30 respectively, and the VL comprises CDRs 1, 2, 3 with the amino acid sequences set forth in SEQ ID NOs: 4-6, respectively, according to the Chothia numbering scheme.

In some embodiments, the VH comprises CDRs 1, 2, 3 with the amino acid sequences set forth in SEQ ID NOs: 31-33, respectively, and the VL comprises CDRs 1, 2, 3 with the amino acid sequences set forth in SEQ ID NOs: 4-6, respectively, according to the Chothia numbering scheme.

In some embodiments, the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 4-6, respectively, according to the Chothia numbering scheme.

In some embodiments, the antibody or antigen-binding fragment specifically binds to human HER2 or canine HER2.

In some embodiments, the antibody or antigen-binding fragment is a human or humanized antibody or antigen-binding fragment thereof.

In some embodiments, the antibody or antigen-binding fragment is a single-chain variable fragment (scFV).

In one aspect, the disclosure is related to an antibody or antigen-binding fragment thereof that binds to HER2 comprising
a heavy chain variable region (VH) comprising an amino acid sequence that is at least 80%, 85%, 90%, 95% or 100% identical to a selected VH sequence, and a light chain variable region (VL) comprising an amino acid sequence that is at least 80%, 85%, 90%, 95% or 100% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37;
(5) the selected VH sequence is SEQ ID NO:42, and the selected VL sequence is SEQ ID NO: 37.

In some embodiments, the VH comprises the sequence of SEQ ID NO: 38 and the VL comprises the sequence of SEQ ID NO: 37.

In some embodiments, the VH comprises the sequence of SEQ ID NO: 39 and the VL comprises the sequence of SEQ ID NO: 37.

In some embodiments, the VH comprises the sequence of SEQ ID NO: 40 and the VL comprises the sequence of SEQ ID NO: 37.

In some embodiments, the VH comprises the sequence of SEQ ID NO: 41 and the VL comprises the sequence of SEQ ID NO: 37.

In some embodiments, the VH comprises the sequence of SEQ ID NO: 42 and the VL comprises the sequence of SEQ ID NO: 37.

In some embodiments, the antibody or antigen-binding fragment specifically binds to human HER2 or canine HER2.

In some embodiments, the antibody or antigen-binding fragment is a human or humanized antibody or antigen-binding fragment thereof.

In some embodiments, the antibody or antigen-binding fragment is a single-chain variable fragment (scFV).

In one aspect, the disclosure is related to an antibody or antigen-binding fragment thereof that cross-competes with the antibody or antigen-binding fragment thereof described herein.

In one aspect, the disclosure is related to an antibody or antigen-binding fragment thereof that binds to HER2 comprising
a heavy chain variable region (VH) comprising VH CDR1, VH CDR2, and VH CDR3 that are identical to VH CDR1, VH CDR2, and VH CDR3 of a selected VH sequence; and
a light chain variable region (VL) comprising VL CDR1, VL CDR2, and VL CDR3 that are identical to VL CDR1, VL CDR2, and VL CDR3 of a selected VL sequence,
wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37;
(5) the selected VH sequence is SEQ ID NO: 42, and the selected VL sequence is SEQ ID NO: 37.

In some embodiments, the antibody or antigen-binding fragment thereof is a bispecific or a multispecific antibody or an antigen-binding fragment thereof.

In some embodiments, the antibody or antigen-binding fragment thereof further specifically binds to TROP2.

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7-9, respectively, and wherein the VH, when paired with a light chain variable region (VL) comprising the amino acid sequence set forth in SEQ ID NO: 37, binds to HER2;
(2) an immunoglobulin heavy chain or a fragment thereof comprising a VH comprising CDRs 1, 2, and 3 comprising the amino acid sequences set forth in SEQ ID NOs: 10-12, respectively, and wherein the VH, when paired with a VL comprising the amino acid sequence set forth in SEQ ID NO: 37, binds to HER2;
(3) an immunoglobulin heavy chain or a fragment thereof comprising a VH comprising CDRs 1, 2, and 3 comprising the amino acid sequences set forth in SEQ ID NOs: 13-15, respectively, and wherein the VH, when paired with a VL comprising the amino acid sequence set forth in SEQ ID NO: 37, binds to HER2;
(4) an immunoglobulin heavy chain or a fragment thereof comprising a VH comprising CDRs 1, 2, and 3 comprising the amino acid sequences set forth in SEQ ID NOs: 16-18, respectively, and wherein the VH, when paired with a VL comprising the amino acid sequence set forth in SEQ ID NO: 37, binds to HER2;
(5) an immunoglobulin heavy chain or a fragment thereof comprising a VH comprising CDRs 1, 2, and 3 comprising the amino acid sequences set forth in SEQ ID NOs: 19-21, respectively, and wherein the VH, when paired with a VL comprising the amino acid sequence set forth in SEQ ID NO: 37 binds to HER2;
(6) an immunoglobulin heavy chain or a fragment thereof comprising a VH comprising CDRs 1, 2, and 3 comprising the amino acid sequences set forth in SEQ ID NOs: 22-24, respectively, and wherein the VH, when paired with a VL comprising the amino acid sequence set forth in SEQ ID NO: 37, binds to HER2;
(7) an immunoglobulin heavy chain or a fragment thereof comprising a VH comprising CDRs 1, 2, and 3 comprising the amino acid sequences set forth in SEQ ID NOs: 25-27, respectively, and wherein the VH, when paired with a VL comprising the amino acid sequence set forth in SEQ ID NO: 37, binds to HER2;
(8) an immunoglobulin heavy chain or a fragment thereof comprising a VH comprising CDRs 1, 2, and 3 comprising the amino acid sequences set forth in SEQ ID NOs: 28-30, respectively, and wherein the VH, when paired with a VL comprising the amino acid sequence set forth in SEQ ID NO: 37, binds to HER2;
(9) an immunoglobulin heavy chain or a fragment thereof comprising a VH comprising CDRs 1, 2, and 3 comprising the amino acid sequences set forth in SEQ ID NOs: 31-33, respectively, and wherein the VH, when paired with a VL comprising the amino acid sequence set forth in SEQ ID NO: 37, binds to HER2;
(10) an immunoglobulin heavy chain or a fragment thereof comprising a VH comprising CDRs 1, 2, and 3 comprising the amino acid sequences set forth in SEQ ID NOs: 34-36, respectively, and wherein the VH, when paired with a VL comprising the amino acid sequence set forth in SEQ ID NO: 37, binds to HER2;
(11) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 38, binds to HER2;
(12) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 39, binds to HER2;
(13) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 40, binds to HER2;
(14) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 41, binds to HER2;
(15) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 42, binds to HER2;
(16) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 38, binds to HER2;
(17) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 39, binds to HER2;
(18) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 40, binds to HER2;
(19) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 41, binds to HER2; or
(20) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 42, binds to HER2.

In some embodiments, the VH when paired with a VL specifically binds to human HER2 or canine HER2.

In some embodiments, the immunoglobulin heavy chain or the fragment thereof is a human or humanized immunoglobulin heavy chain or a fragment thereof.

In some embodiments, the nucleic acid encodes a single-chain variable fragment (scFv).

In some embodiments, the nucleic acid is cDNA.

In one aspect, the disclosure is related to an antibody or antigen-binding fragment thereof that binds to TROP2 (Trophoblast cell-surface antigen 2) comprising:
a heavy chain variable region (VH) comprising complementarity determining regions (CDRs) 1, 2, and 3, wherein the VH CDR1 region comprises an amino acid sequence that is at least 80%, 85%, 90%, 95% or 100% identical to a selected VH CDR1 amino acid sequence, the VH CDR2 region comprises an amino acid sequence that is at least 80%, 85%, 90%, 95% or 100% identical to a selected VH CDR2 amino acid sequence, and the VH CDR3 region comprises an amino acid sequence that is at least 80%, 85%, 90%, 95% or 100% identical to a selected VH CDR3 amino acid sequence; and
a light chain variable region (VL) comprising CDRs 1, 2, and 3, wherein the VL CDR1 region comprises an amino acid sequence that is at least 80%, 85%, 90%, 95% or 100% identical to a selected VL CDR1 amino acid sequence, the VL CDR2 region comprises an amino acid sequence that is at least 80%, 85%, 90%, 95% or 100% identical to a selected VL CDR2 amino acid sequence, and the VL CDR3 region comprises an amino acid sequence that is at least 80%, 85%, 90%, 95% or 100% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 43-45, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 46-48, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 49-51, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 52-54, respectively, and the selected VL CDRs 1, 2, 3 amino acid sequences are set forth in SEQ ID NOs: 4-6, respectively;
(5) the selected VH CDRs 1, 2, 3 amino acid sequences are set forth in SEQ ID NOs: 55-57, respectively, and the selected VL CDRs 1, 2, 3 amino acid sequences are set forth in SEQ ID NOs: 4-6, respectively;
(6) the selected VH CDRs 1, 2, 3 amino acid sequences are set forth in SEQ ID NOs: 58-60, respectively, and the selected VL CDRs 1, 2, 3 amino acid sequences are set forth in SEQ ID NOs: 4-6, respectively.

In some embodiments, the VH comprises CDRs 1, 2, 3 with the amino acid sequences set forth in SEQ ID NOs: 43-45, respectively, and the VL comprises CDRs 1, 2, 3 with the amino acid sequences set forth in SEQ ID NOs: 1-3, respectively, according to the Kabat numbering scheme.

In some embodiments, the VH comprises CDRs 1, 2, 3 with the amino acid sequences set forth in SEQ ID NOs: 46-48, respectively, and the VL comprises CDRs 1, 2, 3 with the amino acid sequences set forth in SEQ ID NOs: 1-3, respectively, according to the Kabat numbering scheme.

In some embodiments, the VH comprises CDRs 1, 2, 3 with the amino acid sequences set forth in SEQ ID NOs: 49-51, respectively, and the VL comprises CDRs 1, 2, 3 with the amino acid sequences set forth in SEQ ID NOs: 1-3, respectively, according to the Kabat numbering scheme.

In some embodiments, the VH comprises CDRs 1, 2, 3 with the amino acid sequences set forth in SEQ ID NOs: 52-54, respectively, and the VL comprises CDRs 1, 2, 3 with the amino acid sequences set forth in SEQ ID NOs: 4-6, respectively, according to the Chothia numbering scheme.

In some embodiments, the VH comprises CDRs 1, 2, 3 with the amino acid sequences set forth in SEQ ID NOs: 55-57, respectively, and the VL comprises CDRs 1, 2, 3 with the amino acid sequences set forth in SEQ ID NOs: 4-6, respectively, according to the Chothia numbering scheme.

In some embodiments, the VH comprises CDRs 1, 2, 3 with the amino acid sequences set forth in SEQ ID NOs: 58-60 respectively, and the VL comprises CDRs 1, 2, 3 with the amino acid sequences set forth in SEQ ID NOs: 4-6, respectively, according to the Chothia numbering scheme.

In some embodiments, the antibody or antigen-binding fragment specifically binds to human TROP2 or canine TROP2.

In some embodiments, the antibody or antigen-binding fragment is a human or humanized antibody or antigen-binding fragment thereof.

In some embodiments, the antibody or antigen-binding fragment is a single-chain variable fragment (scFV).

In one aspect, the disclosure is related to an antibody or antigen-binding fragment thereof that binds to TROP2 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 61, and the selected VL sequence is SEQ ID NO: 37;
(2) the selected VH sequence is SEQ ID NO: 62, and the selected VL sequence is SEQ ID NO: 37;
(3) the selected VH sequence is SEQ ID NO: 63, and the selected VL sequence is SEQ ID NO: 37.

In some embodiments, the VH comprises the sequence of SEQ ID NO: 61 and the VL comprises the sequence of SEQ ID NO: 37.

In some embodiments, the VH comprises the sequence of SEQ ID NO: 62 and the VL comprises the sequence of SEQ ID NO: 37.

In some embodiments, the VH comprises the sequence of SEQ ID NO: 63 and the VL comprises the sequence of SEQ ID NO: 37.

In some embodiments, the antibody or antigen-binding fragment specifically binds to human TROP2 or canine TROP2.

In some embodiments, the antibody or antigen-binding fragment is a human or humanized antibody or antigen-binding fragment thereof.

In some embodiments, the antibody or antigen-binding fragment is a single-chain variable fragment (scFV).

In one aspect, the disclosure is related to an antibody or antigen-binding fragment thereof that cross-competes with the antibody or antigen-binding fragment thereof described herein.

In one aspect, the disclosure is related to an antibody or antigen-binding fragment thereof that binds to TROP2 comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence,
wherein the selected VH sequence and the selected VL sequence are one of the following:
(1) the selected VH sequence is SEQ ID NO: 61, and the selected VL sequence is SEQ ID NO: 37;
(2) the selected VH sequence is SEQ ID NO: 62, and the selected VL sequence is SEQ ID NO: 37;
(3) the selected VH sequence is SEQ ID NO: 63, and the selected VL sequence is SEQ ID NO: 37.

In some embodiments, the antibody or antigen-binding fragment thereof is a bispecific or multispecific antibody or an antigen-binding fragment thereof.

In some embodiments, the antibody or antigen-binding fragment thereof further specifically binds to HER2.

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 43-45, respectively, and wherein the VH, when paired with a light chain variable region (VL) comprising the amino acid sequence set forth in SEQ ID NO: 37, binds to TROP2;
(2) an immunoglobulin heavy chain or a fragment thereof comprising a VH comprising CDRs 1, 2, and 3 comprising the amino acid sequences set forth in SEQ ID NOs: 46-48, respectively, and wherein the VH, when paired with a VL comprising the amino acid sequence set forth in SEQ ID NO: 37, binds to TROP2;
(3) an immunoglobulin heavy chain or a fragment thereof comprising a VH comprising CDRs 1, 2, and 3 comprising the amino acid sequences set forth in SEQ ID NOs: 49-51, respectively, and wherein the VH, when paired with a VL comprising the amino acid sequence set forth in SEQ ID NO: 37, binds to TROP2; or
(4) an immunoglobulin heavy chain or a fragment thereof comprising a VH comprising CDRs 1, 2, and 3 comprising the amino acid sequences set forth in SEQ ID NOs: 52-54, respectively, and wherein the VH, when paired with a VL comprising the amino acid sequence set forth in SEQ ID NO: 37, binds to TROP2;
(5) an immunoglobulin heavy chain or a fragment thereof comprising a VH comprising CDRs 1, 2, and 3 comprising the amino acid sequences set forth in SEQ ID NOs: 55-57, respectively, and wherein the VH, when paired with a VL comprising the amino acid sequence set forth in SEQ ID NO: 37, binds to TROP2;
(6) an immunoglobulin heavy chain or a fragment thereof comprising a VH comprising CDRs 1, 2, and 3 comprising the amino acid sequences set forth in SEQ ID NOs: 58-60, respectively, and wherein the VH, when paired with a VL comprising the amino acid sequence set forth in SEQ ID NO: 37, binds to TROP2;
(7) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 61, binds to TROP2;
(8) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 62, binds to TROP2;
(9) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 63, binds to TROP2;
(10) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 61, binds to TROP2;
(11) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 62, binds to TROP2;
(12) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 63, binds to TROP2.

In some embodiments, the VH when paired with a VL specifically binds to human TROP2 or canine TROP2.

In some embodiments, the immunoglobulin heavy chain or the fragment thereof is a human or humanized immunoglobulin heavy chain or a fragment thereof.

In some embodiments, the nucleic acid encodes a single-chain variable fragment (scFv).

In some embodiments, the nucleic acid is cDNA.

In one aspect, the disclosure is related to an antigen-binding protein construct, comprising: a first antigen-binding domain that specifically binds to HER2; and a second antigen-binding domain that specifically binds to TROP2.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprising complementarity determining regions (CDRs) 1, 2, and 3, wherein the VH1 CDR1 region comprises an amino acid sequence that is at least 80%, 85%, 90%, 95% or 100% identical to a selected VH1 CDR1 amino acid sequence, the VH1 CDR2 region comprises an amino acid sequence that is at least 80%, 85%, 90%, 95% or 100% identical to a selected VH1 CDR2 amino acid sequence, and the VH1 CDR3 region comprises an amino acid sequence that is at least 80%, 85%, 90%, 95% or 100% identical to a selected VH1 CDR3 amino acid sequence; and

the first light chain variable region (VL1) comprising CDRs 1, 2, and 3, wherein the VL1 CDR1 region comprises an amino acid sequence that is at least 80%, 85%, 90%, 95% or 100% identical to a selected VL1 CDR1 amino acid sequence, the VL1 CDR2 region comprises an amino acid sequence that is at least 80%, 85%, 90%, 95% or 100% identical to a selected VL1 CDR2 amino acid sequence, and the VL1 CDR3 region comprises an amino acid sequence that is at least 80%, 85%, 90%, 95% or 100% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOS: 13-15, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 4-6, respectively;
(7) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 4-6, respectively;
(8) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 4-6, respectively;
(9) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 4-6, respectively;
(10) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 4-6, respectively.

In some embodiments, the second heavy chain variable region (VH2) comprising CDRs 1, 2, and 3, wherein the VH2 CDR1 region comprises an amino acid sequence that is at least 80%, 85%, 90%, 95% or 100% identical to a selected VH2 CDR1 amino acid sequence, the VH2 CDR2 region comprises an amino acid sequence that is at least 80%, 85%, 90%, 95% or 100% identical to a selected VH2 CDR2 amino acid sequence, and the VH2 CDR3 region comprises an amino acid sequence that is at least 80%, 85%, 90%, 95% or 100% identical to a selected VH2 CDR3 amino acid sequence; and
the second light chain variable region (VL2) comprising CDRs 1, 2, and 3, wherein the VL2 CDR1 region comprises an amino acid sequence that is at least 80%, 85%, 90%, 95% or 100% identical to a selected VL2 CDR1 amino acid sequence, the VL2 CDR2 region comprises an amino acid sequence that is at least 80%, 85%, 90%, 95% or 100% identical to a selected VL2 CDR2 amino acid sequence, and the VL2 CDR3 region comprises an amino acid sequence that is at least 80%, 85%, 90%, 95% or 100% identical to a selected VL2 CDR3 amino acid sequence, wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOS: 43-45, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(4) the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 4-6, respectively;
(5) the selected VH2 CDRs 1, 2, 3 amino acid sequences are set forth in SEQ ID NOS: 55-57, respectively, and the selected VL2 CDRs 1, 2, 3 amino acid sequences are set forth in SEQ ID NOs: 4-6, respectively;
(6) the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 4-6, respectively.

In some embodiments,
(1) the selected VH1 CDRs 1, 2, 3 amino acid sequences are set forth in SEQ ID NOS: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(2) the selected VH1 CDRs 1, 2, 3 amino acid sequences are set forth in SEQ ID NOS: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(6) the selected VH1 CDRs 1, 2, 3 amino acid sequences are set forth in SEQ ID NOS: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(15) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively.

In some embodiments, the first heavy chain variable region comprises a sequence that is at least 80%, 85%, 90%, 95% or 100% identical to SEQ ID NO: 39, the first light chain variable region comprises a sequence that is at least 80%, 85%, 90%, 95% or 100% identical to SEQ ID NO: 37, the second heavy chain variable region comprises a sequence that is at least 80%, 85%, 90%, 95% or 100% identical to SEQ ID NO: 63, and the second light chain variable region comprises a sequence that is at least 80%, 85%, 90%, 95% or 100% identical to SEQ ID NO: 37.

In some embodiments, the first heavy chain variable region comprises a sequence that is at least 80%, 85%, 90%, 95% or 100% identical to SEQ ID NO: 42, the first light chain variable region comprises a sequence that is at least 80%, 85%, 90%, 95% or 100% identical to SEQ ID NO: 37, the second heavy chain variable region comprises a sequence that is at least 80%, 85%, 90%, 95% or 100% identical to SEQ ID NO: 63, and the second light chain variable region comprises a sequence that is at least 80%, 85%, 90%, 95% or 100% identical to SEQ ID NO: 37.

In some embodiments, the first heavy chain variable region comprises a sequence that is at least 80%, 85%, 90%, 95% or 100% identical to SEQ ID NO: 38, the first light chain variable region comprises a sequence that is at least 80%, 85%, 90%, 95% or 100% identical to SEQ ID NO: 37, the second heavy chain variable region comprises a sequence that is at least 80%, 85%, 90%, 95% or 100% identical to SEQ ID NO: 63, and the second light chain variable region comprises a sequence that is at least 80%, 85%, 90%, 95% or 100% identical to SEQ ID NO: 37.

In some embodiments, the first heavy chain variable region comprises a sequence that is at least 80%, 85%, 90%, 95% or 100% identical to SEQ ID NO: 38, the first light chain variable region comprises a sequence that is at least 80%, 85%, 90%, 95% or 100% identical to SEQ ID NO: 37, the second heavy chain variable region comprises a sequence that is at least 80%, 85%, 90%, 95% or 100% identical to SEQ ID NO: 62, and the second light chain variable region comprises a sequence that is at least 80%, 85%, 90%, 95% or 100% identical to SEQ ID NO: 37.

In some embodiments, the first heavy chain variable region comprises a sequence that is at least 80%, 85%, 90%, 95% or 100% identical to SEQ ID NO: 39, the first light chain variable region comprises a sequence that is at least 80%, 85%, 90%, 95% or 100% identical to SEQ ID NO: 37, the second heavy chain variable region comprises a sequence that is at least 80%, 85%, 90%, 95% or 100% identical to SEQ ID NO: 62, and the second light chain variable region comprises a sequence that is at least 80%, 85%, 90%, 95% or 100% identical to SEQ ID NO: 37.

In some embodiments, the first heavy chain variable region comprises a sequence that is at least 80%, 85%, 90%, 95% or 100% identical to SEQ ID NO: 42, the first light chain variable region comprises a sequence that is at least 80%, 85%, 90%, 95% or 100% identical to SEQ ID NO: 37, the second heavy chain variable region comprises a sequence that is at least 80%, 85%, 90%, 95% or 100% identical to SEQ ID NO: 62, and the second light chain variable region comprises a sequence that is at least 80%, 85%, 90%, 95% or 100% identical to SEQ ID NO: 37.

In some embodiments, the first heavy chain variable region comprises a sequence that is at least 80%, 85%, 90%, 95% or 100% identical to SEQ ID NO: 38, the first light chain variable region comprises a sequence that is at least 80%, 85%, 90%, 95% or 100% identical to SEQ ID NO: 37, the second heavy chain variable region comprises a sequence that is at least 80%, 85%, 90%, 95% or 100% identical to SEQ ID NO: 61, and the second light chain variable region comprises a sequence that is at least 80%, 85%, 90%, 95% or 100% identical to SEQ ID NO: 37.

In some embodiments, the first heavy chain variable region comprises a sequence that is at least 80%, 85%, 90%, 95% or 100% identical to SEQ ID NO: 39, the first light chain variable region comprises a sequence that is at least 80%, 85%, 90%, 95% or 100% identical to SEQ ID NO: 37, the second heavy chain variable region comprises a sequence that is at least 80%, 85%, 90%, 95% or 100% identical to SEQ ID NO: 61, and the second light chain variable region comprises a sequence that is at least 80%, 85%, 90%, 95% or 100% identical to SEQ ID NO: 37.

In some embodiments, the first heavy chain variable region comprises a sequence that is at least 80%, 85%, 90%, 95% or 100% identical to SEQ ID NO: 42, the first light chain variable region comprises a sequence that is at least 80%, 85%, 90%, 95% or 100% identical to SEQ ID NO: 37, the second heavy chain variable region comprises a sequence that is at least 80%, 85%, 90%, 95% or 100% identical to SEQ ID NO: 61, and the second light chain variable region comprises a sequence that is at least 80%, 85%, 90%, 95% or 100% identical to SEQ ID NO: 37.

In some embodiments, the first heavy chain variable region comprises a sequence that is at least 80%, 85%, 90%, or 95% identical to SEQ ID NO: 40, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 63, and the second light chain variable region comprises a sequence that is at least 80%, 85%, 90%, or 95% identical to SEQ ID NO: 37.

In some embodiments, the first heavy chain variable region comprises a sequence that is at least 80%, 85%, 90%, or 95% identical to SEQ ID NO: 41, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 63, and the second light chain variable region comprises a sequence that is at least 80%, 85%, 90%, or 95% identical to SEQ ID NO: 37.

In some embodiments, the first heavy chain variable region comprises a sequence that is at least 80%, 85%, 90%, or 95% identical to SEQ ID NO: 40, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 62, and the second light chain variable region comprises a sequence that is at least 80%, 85%, 90%, or 95% identical to SEQ ID NO: 37.

In some embodiments, the first heavy chain variable region comprises a sequence that is at least 80%, 85%, 90%, or 95% identical to SEQ ID NO: 41, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 62, and the second light chain variable region comprises a sequence that is at least 80%, 85%, 90%, or 95% identical to SEQ ID NO: 37.

In some embodiments, the first heavy chain variable region comprises a sequence that is at least 80%, 85%, 90%, or 95% identical to SEQ ID NO: 40, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 61, and the second light chain variable region comprises a sequence that is at least 80%, 85%, 90%, or 95% identical to SEQ ID NO: 37.

In some embodiments, the first heavy chain variable region comprises a sequence that is at least 80%, 85%, 90%, or 95% identical to SEQ ID NO: 41, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 61, and the second light chain variable region comprises a sequence that is at least 80%, 85%, 90%, or 95% identical to SEQ ID NO: 37.

In some embodiments, the antigen-binding protein construct is a bispecific antibody.

In some embodiments, the first light chain variable region and the second light chain variable region are identical.

In one aspect, the disclosure is related to a vector comprising one or more of the nucleic acids described herein, or a nucleic acid encoding the antibody or antigen-binding fragment thereof described herein, or a nucleic acid encoding the antigen-binding protein construct described herein.

In one aspect, the disclosure is related to a cell comprising the vector described herein.

In some embodiments, the cell is a CHO cell.

In one aspect, the disclosure is related to a cell comprising one or more of the nucleic acids described herein, or a nucleic acid encoding the antibody or antigen-binding fragment thereof described herein, or a nucleic acid encoding the antigen-binding protein construct described herein.

In one aspect, the disclosure is related to a method of producing an antibody or an antigen-binding fragment thereof, or an antigen-binding protein construct, the method comprising
(a) culturing the cell described herein under conditions sufficient for the cell to produce the antibody or the antigen-binding fragment thereof, or the antigen-binding protein construct; and
(b) collecting the antibody or the antigen-binding fragment thereof, or the antigen-binding protein construct produced by the cell.

In one aspect, the disclosure is related to an antibody-drug conjugate comprising a therapeutic agent covalently bound to:
(a) the antibody or antigen-binding fragment thereof described herein; or
(b) the antigen-binding protein construct described herein.

In some embodiments, the therapeutic agent is a cytotoxic or cytostatic agent.

In some embodiments, the therapeutic agent is MMAE or MMAF.

In one aspect, the disclosure is related to a method of treating a subject having cancer, the method comprising administering a therapeutically effective amount of a composition comprising the antibody or antigen-binding fragment thereof described herein, the antigen-binding protein construct described herein, or the antibody-drug conjugate described herein, to the subject.

In some embodiments, the subject has a solid tumor.

In some embodiments, the cancer is thyroid cancer, urothelial cancer, breast cancer, colorectal cancer, renal cancer, cervical cancer, ovarian cancer, lung cancer, endometrial cancer, skin cancer, stomach cancer, pancreatic cancer, prostate cancer, liver cancer, lymphoma, or glioma.

In some embodiments, the cancer is cervical cancer, prostate cancer, thyroid cancer, urothelial cancer, head and neck cancer, endometrial cancer, ovarian cancer, lung cancer, breast cancer, carcinoid, skin cancer, liver cancer, or testis cancer.

In some embodiments, the cancer is multiple myeloma or renal carcinoma.

In some embodiments, the subject is a human.

In some embodiments, the subject is a non-human animal.

In one aspect, the disclosure is related to a method of decreasing the rate of tumor growth, the method comprising contacting a tumor cell with an effective amount of a composition comprising the antibody or antigen-binding fragment thereof described herein, the antigen-binding protein construct described herein, or the antibody-drug conjugate described herein.

In one aspect, the disclosure is related to a method of killing a tumor cell, the method comprising contacting a tumor cell with an effective amount of a composition comprising the antibody or antigen-binding fragment thereof described herein, the antigen-binding protein construct described herein, or the antibody-drug conjugate described herein.

In one aspect, the disclosure is related to a pharmaceutical composition comprising a pharmaceutically acceptable carrier and
(a) the antibody or antigen-binding fragment thereof described herein,
(b) the antigen-binding protein construct described herein, or
(c) the antibody-drug conjugate described herein.

In one aspect, the disclosure relates to an antigen-binding protein construct, including a first antigen-binding domain that specifically binds to HER2; and a second antigen-binding domain that specifically binds to TROP2.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “human antibody” refers to an antibody that is encoded by an endogenous nucleic acid (e.g., rearranged human immunoglobulin heavy or light chain locus) derived from a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different species (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “antigen-binding protein construct” is (i) a single polypeptide that includes at least one antigen-binding domain or (ii) a complex of two or more polypeptides (e.g., the same or different polypeptides) that together form at least one or more different antigen-binding domains. Non-limiting examples and aspects of antigen-binding protein constructs are described herein. Additional examples and aspects of antigen-binding protein constructs are known in the art. In some embodiments, the antigen-binding protein construct has 1, 2, 3, 4, 5, 6, 7, 8, or more than 8 antigen-binding domains.

As used herein, the term “antigen-binding domain” refers to one or more protein domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. One example of an antigen-binding domain is an antigen-binding domain formed by a VH-VL dimer. In some embodiments, the antigen-binding domain is a VHH. Non-limiting examples of antigen-binding domains are described herein. Additional examples of antigen-binding domains are known in the art. In some examples, an antigen-binding domain can bind to a single antigen.

As used herein, the term “bispecific antibody” refers to an antibody that binds to two different epitopes. The epitopes can be on the same antigen or on different antigens.

As used herein, the term “multispecific antibody” refers to an antibody that binds to two or more different epitopes. The epitopes can be on the same antigen or on different antigens.

As used herein, a “VHH” refers to the variable domain of a heavy chain antibody. In some embodiments, the VHH is a humanized VHH.

As used herein, the term “common light chain” refers to a light chain that can interact with two or more different heavy chains, forming different antigen-binding domains, wherein these different antigen-binding domains can specifically bind to different antigens or epitopes. Similarly, the term “common light chain variable region” refers to a light chain variable region that can interact with two or more different heavy chain variable regions, forming different antigen-binding domains, wherein these different antigen-binding domains can specifically bind to different antigens or epitopes. In some embodiments, the antigen-binding construct can have a common light chain. In some embodiments, the antigen-binding construct can have a common light chain variable region.

As used herein, when referring to an antibody, the phrases “specifically binding” and “specifically binds” mean that the antibody interacts with its target molecule (e.g., HER2) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a HER2 molecule may be referred to as a HER2-specific antibody or an anti-HER2 antibody.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing the structure of an exemplary anti-HER2/TROP2 bispecific antibody.

FIG. 2 shows the endocytosis results of the purified antibodies in NCI-N87 cells.

FIG. 3 shows the endocytosis results of the purified antibodies in NCI-N87 cells.

FIG. 4 shows the endocytosis results of the purified antibodies in NCI-H292 cells.

FIG. 5 shows the endocytosis results of the purified antibodies in NCI-H292 cells.

FIG. 6 shows accelerated stability of anti-HER2/TROP2 bispecific antibodies H-1H2-T-6F7, H-2B2-T-6F7, T-6F7-H-1H2 and H-3C8-T-6F7. ND: not detected. Freeze 1: freeze-thaw once; freeze 10: freeze-thaw repeatedly 10 times.

FIG. 7 is a graph showing average tumor volume in different groups of mice that were injected with lung adenocarcinoma cells, and were treated with different Antibody Drug Conjugates (ADC).

FIG. 8 lists CDR sequences of anti-HER2 antibodies H-1H2, H-2B2, H-3E5, H-3C6 and H-3C8 as defined by Kabat numbering.

FIG. 9 lists CDR sequences of anti-HER2 antibodies H-1H2, H-2B2, H-3E5, H-3C6 and H-3C8 as defined by Chothia numbering.

FIG. 10 lists amino acid sequences of heavy chain variable regions and light chain variable regions of anti-HER2 antibodies.

FIG. 11 lists CDR sequences of anti-TROP2 antibodies T-3A4, T-4B9, T-4C12, T-5C8 and T-6F7 as defined by Kabat numbering.

FIG. 12 lists CDR sequences of anti-TROP2 antibodies T-3A4, T-4B9, T-4C12, T-5C8 and T-6F7 as defined by Chothia numbering.

FIG. 13 lists amino acid sequences of heavy chain variable regions and light chain variable regions of anti-TROP2 antibodies.

FIG. 14 lists amino acid sequences as described in the present disclosure.

FIG. 15 is a graph showing average tumor volumes in different groups of mice that were injected with lung adenocarcinoma cells, and were treated with PBS, MMAE, H-2B2-T-6F7 or H-2B2-T-6F7-ADC.

FIG. 16 is a graph showing average tumor volumes in different groups of mice that were injected with lung adenocarcinoma cells, and were treated with PBS, H-2B2-T-6F7-ADC, Sacituzumab govitecan analog, Disitamab vedotin or Trastuzumab deruxtecan.

FIG. 17 is a graph showing average tumor volumes in different groups of mice that were injected with ovarian cancer cells, and were treated with PBS, Isotype-Control-ADC, H-2B2-T-6F7-ADC, H-2B2-IgG1-ADC, T-6F7-IgG1-SI-ADC, Trastuzumab deruxtecan analog or DS-1062 analog.

FIG. 18 is a graph showing average tumor volumes in different groups of mice that were injected with breast cancer cells, and were treated with PBS, Isotype-Control-ADC, H-2B2-T-6F7-ADC, H-2B2-IgG1-ADC, T-6F7-IgG1-SI-ADC, Sacituzumab govitecan analog, Disitamab vedotin or Trastuzumab deruxtecan.

FIG. 19 is a graph showing average tumor volumes in different groups of mice that were injected with patient-derived pancreatic tumor fragments, and were treated with PBS, H-2B2-T-6F7-ADC, T-6F7-IgG1-SI-ADC, Sacituzumab govitecan analog, Disitamab vedotin or Trastuzumab deruxtecan at 5 mg/kg.

FIG. 20 is a graph showing average tumor volumes in different groups of mice that were injected with patient-derived pancreatic tumor fragments, and were treated with PBS, H-2B2-T-6F7-ADC, T-6F7-IgG1-SI-ADC, Sacituzumab govitecan analog, Disitamab vedotin or Trastuzumab deruxtecan at 3 mg/kg.

FIG. 21A is a graph showing average body weight in different groups of mice that were administered with physiological saline or H-2B2-T-6F7-ADC.

FIG. 21B is a graph showing average body weight changes in different groups of mice that were administered with physiological saline or H-2B2-T-6F7-ADC.

FIG. 22 is a graph showing average tumor volumes in different groups of mice that were injected with patient-derived colorectal tumor fragments, and were treated with PBS, 6 mg/kg H-2B2-T-6F7-ADC, 10 mg/kg Sacituzumab govitecan, 6 mg/kg Disitamab vedotin or 6 mg/kg Trastuzumab deruxtecan at 6 mg/kg.

FIG. 23 is a graph showing average tumor volumes in different groups of mice that were injected with patient-derived lung tumor fragments, and were treated with PBS, 3 mg/kg or 6 mg/kg H-2B2-T-6F7-ADC, 3 mg/kg H-2B2-IgG1-ADC, 3 mg/kg T-6F7-IgG1-SI-ADC, 10 mg/kg Sacituzumab govitecan, 6 mg/kg Disitamab vedotin or 6 mg/kg Trastuzumab deruxtecan.

FIG. 24 is a graph showing average tumor volumes in different groups of mice that were injected with patient-derived gastric tumor fragments, and were treated with PBS, 3 mg/kg or 6 mg/kg H-2B2-T-6F7-ADC, 10 mg/kg Sacituzumab govitecan, 6 mg/kg Disitamab vedotin or 6 mg/kg Trastuzumab deruxtecan.

FIG. 25 shows the results of plasma stability test for H-2B2-T-6F7-ADC of the present disclosure.

### DETAILED DESCRIPTION

A bispecific antibody or antigen-binding fragment thereof is an artificial protein that can simultaneously bind to two different epitopes (e.g., on two different antigens). In some embodiments, a bispecific antibody or antigen-binding fragment thereof can have two arms (Arms A and B). Each arm has one heavy chain variable region and one light chain variable region, forming an antigen-binding domain (or an antigen-binding region). In some embodiments, the bispecific antibody has a common light chain.

The present disclosure relates to anti-HER2 antibodies or antigen binding fragments thereof, anti-TROP2 antibodies or antigen binding fragments thereof, antigen-binding protein constructs (e.g., bispecific antibodies or antigen-binding fragments thereof) that specifically bind to two different antigens (e.g., HER2 and TROP2), and antibody drug conjugates.

### Anti-HER2 Antibodies and Antigen-Binding Fragments

Human epidermal growth factor receptor 2 (HER2) (also known as ERBB2) is a transmembrane receptor belonging to the epidermal growth factor receptor subfamily of receptor protein tyrosine kinases.

HER2 is overexpressed in various cancer types such as breast cancer and gastric cancer and has been reported to be a negative prognostic factor in breast cancer. As anti-HER2 drugs effective for HER2-overexpressing cancers, trastuzumab, trastuzumab emtansine, pertuzumab, lapatinib, and the like are known.

The disclosure provides several antibodies and antigen-binding fragments thereof that specifically bind to HER2. In some embodiments, the anti-HER2/TROP2 antigen-binding protein constructs (e.g., bispecific antibodies) can include an antigen binding region that is derived from these antibodies.

The antibodies and antigen-binding fragments described herein are capable of binding to HER2. The disclosure provides e.g., anti-HER2 antibodies H-1H2 (“1H2”), H-2B2 (“2B2”), H-3E5 (“3E5”), H-3C6 (“3C6”), H-3C8 (“3C8”), and the antibodies derived therefrom.

The CDR sequences for 1H2, and 1H2 derived antibodies (e.g., human antibodies) include CDRs of the heavy chain variable domain, SEQ ID NOs: 7-9, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 22-24, and CDR sequences of the light chain variable domain are set forth in SEQ ID NOs: 4-6.

Similarly, the CDR sequences for 2B2, and 2B2 derived antibodies include CDRs of the heavy chain variable domain, SEQ ID NOs: 10-12, and CDRs of the light chain variable domain, SEQ ID NOs: 1-3, as defined by Kabat numbering. Under Chothia numbering, the CDR sequences of the heavy chain variable domain are set forth in SEQ ID NOs: 25-27, and CDRs of the light chain variable domain are set forth in SEQ ID NOs: 4-6.

The CDR sequences for 3E5, and 3E5 derived antibodies include CDRs of the heavy chain variable domain, SEQ ID NOs: 13-15, and CDRs of the light chain variable domain, SEQ ID NOs: 1-3, as defined by Kabat numbering. Under Chothia numbering, the CDR sequences of the heavy chain variable domain are set forth in SEQ ID NOs: 28-30, and CDRs of the light chain variable domain are set forth in SEQ ID NOs: 4-6.

The CDR sequences for 3C6, and 3C6 derived antibodies include CDRs of the heavy chain variable domain, SEQ ID NOs: 16-18, and CDRs of the light chain variable domain, SEQ ID NOs: 1-3, as defined by Kabat numbering. Under Chothia numbering, the CDR sequences of the heavy chain variable domain are set forth in SEQ ID NOs: 31-33, and CDRs of the light chain variable domain are set forth in SEQ ID NOs: 4-6.

The CDR sequences for 3C8, and 3C8 derived antibodies include CDRs of the heavy chain variable domain, SEQ ID NOs: 19-21, and CDRs of the light chain variable domain, SEQ ID NOs: 1-3, as defined by Kabat numbering. Under Chothia numbering, the CDR sequences of the heavy chain variable domain are set forth in SEQ ID NOs: 34-36, and CDRs of the light chain variable domain are set forth in SEQ ID NOs: 4-6.

Furthermore, in some embodiments, the antibodies or antigen-binding fragments thereof described herein can also contain one, two, or three heavy chain variable region CDRs selected from the group of SEQ ID NOs: 7-9, SEQ ID NOs: 10-12, SEQ ID NOs: 13-15, SEQ ID NOS: 16-18, SEQ ID NOs: 19-21, SEQ ID NOs: 22-24, SEQ ID NOs: 25-27, SEQ ID NOs: 28-30, SEQ ID NOs: 31-33 and SEQ ID NOs: 34-36; and/or one, two, or three light chain variable region CDRs selected from the group of SEQ ID NOs: 1-3, and SEQ ID NOs: 4-6.

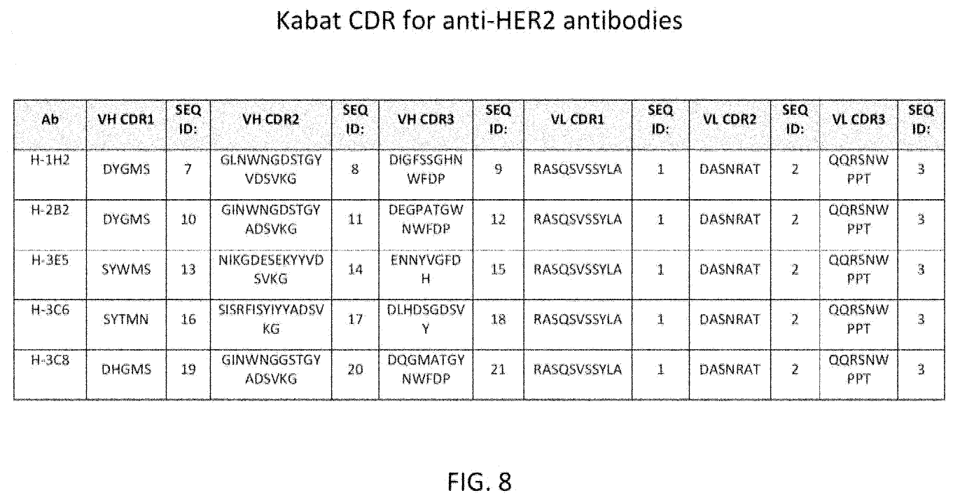

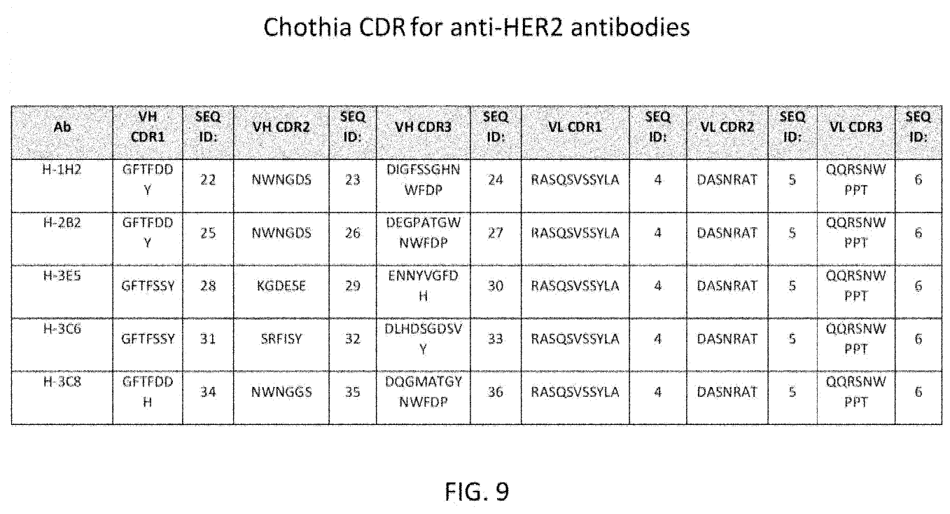

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 8 (Kabat CDR) and FIG. 9 (Chothia CDR).

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a light chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that bind to HER2. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NOs: 38, 39, 40, 41, or 42, and the selected VL sequence is SEQ ID NOs: 37.

In some embodiments, the antibody or antigen binding fragment thereof can have 3 VH CDRs that are identical to the CDRs of any VH sequences as described herein. In some embodiments, the antibody or antigen binding fragment thereof can have 3 VL CDRs that are identical to the CDRs of any VL sequences as described herein.

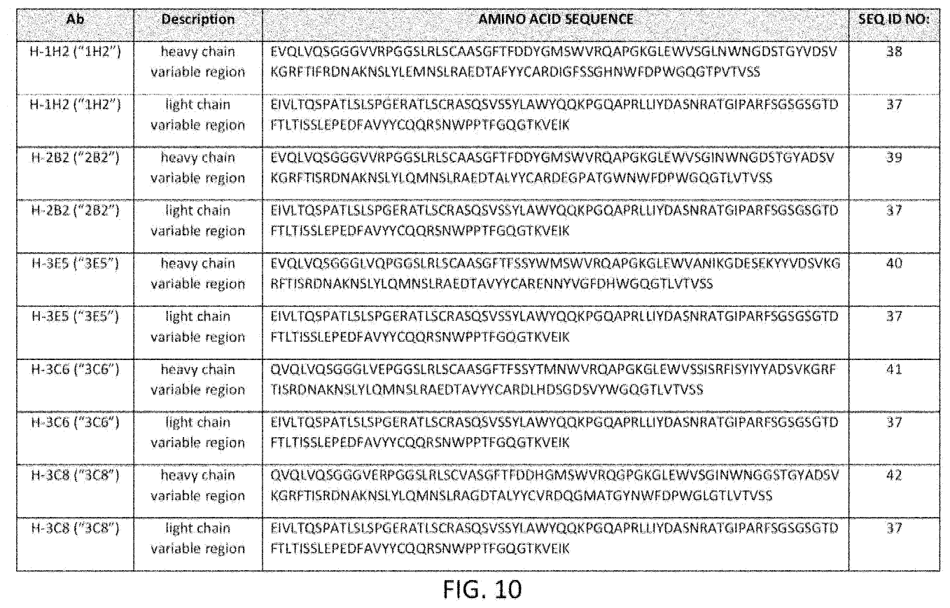

The disclosure also provides nucleic acid comprising a polynucleotide encoding a polypeptide comprising an immunoglobulin heavy chain or an immunoglobulin heavy chain. The immunoglobulin heavy chain or immunoglobulin light chain comprises CDRs as shown in FIG. 8 or FIG. 9, or have sequences as shown in FIG. 10. When the polypeptides are paired with corresponding polypeptide (e.g., a corresponding heavy chain variable region or a corresponding light chain variable region), the paired polypeptides bind to HER2 (e.g., human HER2).

The anti-HER2 antibodies and antigen-binding fragments can also be antibody variants (including derivatives and conjugates) of antibodies or antibody fragments and multi-specific (e.g., bispecific) antibodies or antibody fragments. Additional antibodies provided herein are polyclonal, monoclonal, multi-specific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to HER2 will retain an ability to bind to HER2. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

### Anti-TROP2 Antibodies and Antigen-Binding Fragments

Trophoblast cell-surface antigen 2 (TROP2), also known as Tumor-associated calcium signal transducer 2 (TACSTD2), is a cell surface glycoprotein encoded and expressed by the TACSTD2 gene. It has high structural sequence similarity with epithelial adhesion molecule Epcam. TROP2 is a protein closely related to tumors. It mainly promotes tumor cell growth, proliferation and metastasis by regulating calcium ion signaling pathways, cyclin expression, and reducing fibronectin adhesion. Studies have found that TROP2 protein is highly expressed in breast cancer, colon cancer, bladder cancer, gastric cancer, oral squamous cell carcinoma and ovarian cancer. The protein can promote tumor cell proliferation, invasion, metastasis, spread and other processes. In addition, in breast cancer and other cancers, the high expression of TROP2 has also been found to be closely related to more aggressive diseases and poor clinical prognosis of tumors.

The disclosure provides antibodies and antigen-binding fragments thereof that specifically bind to TROP2. The anti-HER2/TROP2 antigen-binding protein construct (e.g., bispecific antibodies) can include an antigen binding region that is derived from these antibodies.

The antibodies and antigen-binding fragments described herein are capable of binding to TROP2. The disclosure provides anti-TROP2 antibodies T-3A4 (“3A4”), T-4B9 (“4B9”), and T-6F7 (“6F7”), and the antibodies derived therefrom.

The CDR sequences for 3A4, and 3A4 derived antibodies (e.g., human antibodies) include CDRs of the heavy chain variable domain, SEQ ID NOs: 43-45, and CDRs of the light chain variable domain, SEQ ID NOs: 1-3 as defined by Kabat numbering. The CDRs can also be defined by Chothia system. Under the Chothia numbering, the CDR sequences of the heavy chain variable domain are set forth in SEQ ID NOs: 52-54, and CDR sequences of the light chain variable domain are set forth in SEQ ID NOs: 4-6.

Similarly, the CDR sequences for 4B9, and 4B9 derived antibodies include CDRs of the heavy chain variable domain, SEQ ID NOs: 46-48, and CDRs of the light chain variable domain, SEQ ID NOs: 1-3, as defined by Kabat numbering. Under Chothia numbering, the CDR sequences of the heavy chain variable domain are set forth in SEQ ID NOs: 55-57, and CDRs of the light chain variable domain are set forth in SEQ ID NOs: 4-6.

The CDR sequences for 6F7, and 6F7 derived antibodies include CDRs of the heavy chain variable domain, SEQ ID NOs: 49-51, and CDRs of the light chain variable domain, SEQ ID NOs: 1-3, as defined by Kabat numbering. Under Chothia numbering, the CDR sequences of the heavy chain variable domain are set forth in SEQ ID NOs: 58-60, and CDRs of the light chain variable domain are set forth in SEQ ID NOs: 4-6.

Furthermore, in some embodiments, the antibodies or antigen-binding fragments thereof described herein can also contain one, two, or three heavy chain variable region CDRs selected from the group of SEQ ID NOs: 43-45, SEQ ID NOs: 46-48, SEQ ID NOs: 49-51, SEQ ID NOs: 52-54, SEQ ID NOs: 55-57, SEQ ID NOs: 58-60; and/or one, two, or three light chain variable region CDRs selected from the group of SEQ ID NOs: 1-3, SEQ ID NOs: 4-6.

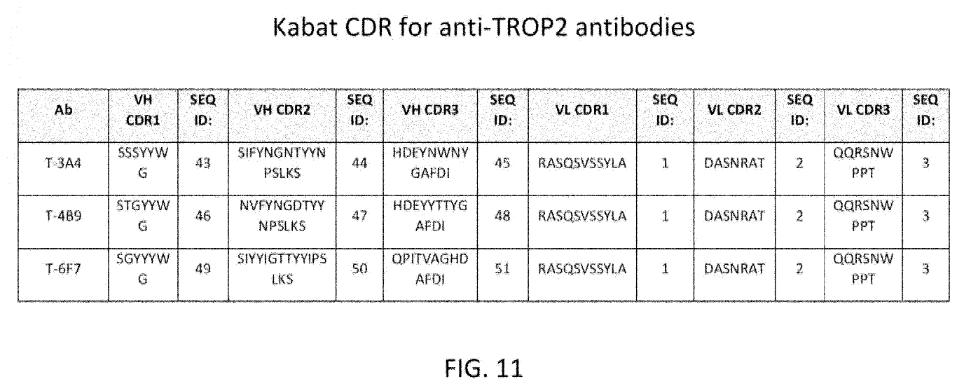

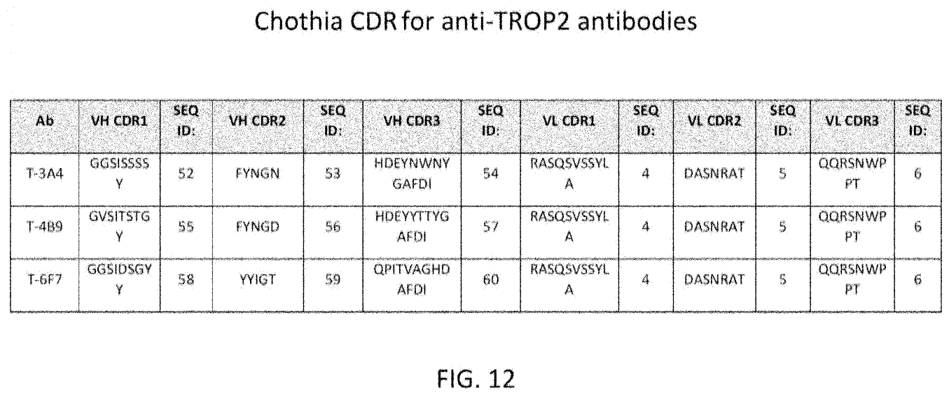

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence, and a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 11 (Kabat CDR) and FIG. 12 (Chothia CDR).

In some embodiments, the antibody or an antigen-binding fragment described herein can contain a heavy chain variable domain containing one, two, or three of the CDRs of SEQ ID NO: 43 with zero, one or two amino acid insertions, deletions, or substitutions; SEQ ID NO: 44 with zero, one or two amino acid insertions, deletions, or substitutions; SEQ ID NO: 45 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 46 with zero, one or two amino acid insertions, deletions, or substitutions; SEQ ID NO: 47 with zero, one or two amino acid insertions, deletions, or substitutions; SEQ ID NO: 48 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 49 with zero, one or two amino acid insertions, deletions, or substitutions; SEQ ID NO: 50 with zero, one or two amino acid insertions, deletions, or substitutions; SEQ ID NO: 51 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 52 with zero, one or two amino acid insertions, deletions, or substitutions; SEQ ID NO: 53 with zero, one or two amino acid insertions, deletions, or substitutions; SEQ ID NO: 54 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 55 with zero, one or two amino acid insertions, deletions, or substitutions; SEQ ID NO: 56 with zero, one or two amino acid insertions, deletions, or substitutions; SEQ ID NO: 57 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 58 with zero, one or two amino acid insertions, deletions, or substitutions; SEQ ID NO: 59 with zero, one or two amino acid insertions, deletions, or substitutions; SEQ ID NO: 60 with zero, one or two amino acid insertions, deletions, or substitutions.

In some embodiments, the antibody or an antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a light chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that binds to TROP2. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 61, 62 or 63, and the selected VL sequence is SEQ ID NO: 37.

In some embodiments, the antibody or antigen binding fragments thereof can have 3 VH CDRs that are identical to the CDRs of any VH sequences as described herein. In some embodiments, the antibody or antigen binding fragments thereof can have 3 VL CDRs that are identical to the CDRs of any VL sequences as described herein.

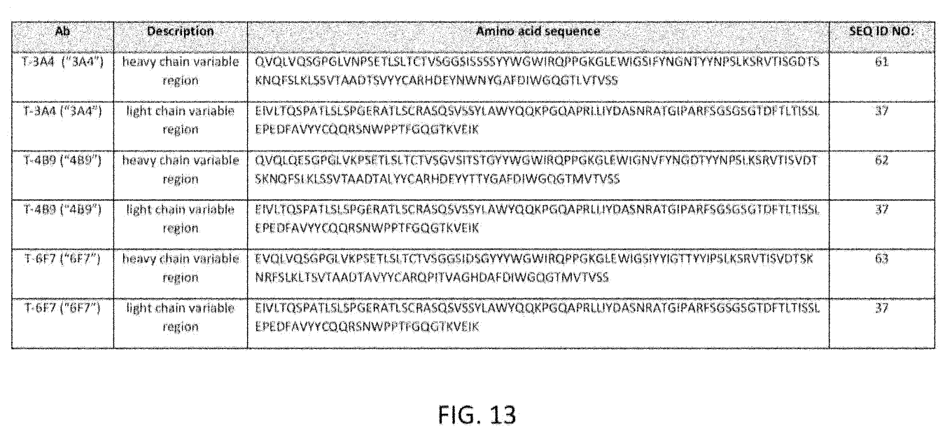

The disclosure also provides nucleic acid comprising a polynucleotide encoding a polypeptide comprising an immunoglobulin heavy chain or an immunoglobulin heavy chain. The immunoglobulin heavy chain or immunoglobulin light chain comprises CDRs as shown in FIG. 11 or FIG. 12, or have sequences as shown in FIG. 13. When the polypeptides are paired with corresponding polypeptide (e.g., a corresponding heavy chain variable region or a corresponding light chain variable region), the paired polypeptides bind to TROP2.

The anti-TROP2 antibodies and antigen-binding fragments can also be antibody variants (including derivatives and conjugates) of antibodies or antibody fragments and multi-specific (e.g., bispecific) antibodies or antibody fragments. Additional antibodies provided herein are polyclonal, monoclonal, multi-specific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to TROP2 will retain an ability to bind to TROP2. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

### Antibodies, Antigen Binding Fragments and Antigen Binding Protein Constructs

The present disclosure provides antibodies, the antigen-binding fragments thereof, or the antigen-binding protein constructs (e.g., bispecific antibodies). The antigen-binding protein construct (e.g., bispecific antibody) can comprise an anti-HER2 antibody or antigen-binding fragment thereof, and anti-TROP2 antibody or antigen-binding fragment thereof. These antigen-binding protein constructs (e.g., bispecific antibody), anti-HER2 antibodies, anti-TROP2 antibodies, and antigen-binding fragments thereof can have various forms.

In general, antibodies (also called immunoglobulins) can be made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68 (1-3): 9-16 (October 1997); Morea et al., J Mol Biol. 275 (2): 269-94 (January 1998); Chothia et al., Nature 342 (6252): 877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The antigen binding domain or antigen binding fragment is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFV has two heavy chain variable domains, and two light chain variable domains. In some embodiments, the scFV has two antigen binding regions (Antigen binding regions: A and B), and the two antigen binding regions can bind to the respective target antigens with different affinities.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the antibodies, the antigen-binding fragments thereof, or the antigen-binding protein constructs (e.g., bispecific antibodies) can bind to two different antigens or two different epitopes.

In some embodiments, the antibodies, the antigen-binding fragments thereof, or the antigen-binding protein constructs (e.g., bispecific antibodies) can comprises one, two, or three heavy chain variable region CDRs selected from FIGS. 8, 9, 11, and 12. In some embodiments, the antibodies, the antigen-binding fragments thereof, or the antigen-binding protein constructs (e.g., bispecific antibodies) can comprises one, two, or three light chain variable region CDRs selected from FIGS. 8, 9, 11, and 12.

In some embodiments, the antibodies, the antigen-binding fragments thereof, or the antigen-binding protein constructs (e.g., bispecific antibodies) described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

In some embodiments, the multi-specific antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the antibodies, the antigen-binding fragments thereof, or the antigen-binding protein constructs (e.g., bispecific antibodies) described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

The antibodies, the antigen-binding fragments thereof, or the antigen-binding protein constructs (e.g., bispecific antibodies) can also have various forms. Many different formats of antigen binding constructs are known in the art, and are described e.g., in Suurs, et al. “A review of bispecific antibodies and antibody constructs in oncology and clinical challenges,” Pharmacology & therapeutics (2019), which is incorporated herein by reference in the entirety.

In some embodiments, the antigen-binding protein construct is a BiTe, a (scFv)2, a nanobody, a nanobody-HSA, a DART, a TandAb, a scDiabody, a scDiabody-CH3, scFv-CH-CL-scFv, a HSAbody, scDiabody-HAS, or a tandem-scFv. In some embodiments, the antigen-binding protein construct is a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a KA-body, an orthogonal Fab, a DVD-IgG, a IgG (H)-scFv, a scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, or a scFv1-PEG-scFv2.

In some embodiments, the antigen-binding protein construct can be a TrioMab. In a TrioMab, the two heavy chains are from different species, wherein different sequences restrict the heavy-light chain pairing.

In some embodiments, the antigen-binding protein construct has two different heavy chains and one common light chain. Heterodimerization of heavy chains can be based on the knob-in-holes or some other heavy chain pairing technique.

In some embodiments, CrossMAb technique can be used produce bispecific antibodies. CrossMAb technique can be used enforce correct light chain association in bispecific heterodimeric IgG antibodies, this technique allows the generation of various bispecific antibody formats, including bi-(1+1), tri-(2+1) and tetra-(2+2) valent bispecific antibodies, as well as non-Fc tandem antigen-binding fragment (Fab)-based antibodies. These formats can be derived from any existing antibody pair using domain crossover, without the need for the identification of common light chains, post-translational processing/in vitro chemical assembly or the introduction of a set of mutations enforcing correct light chain association. The method is described in Klein et al., “The use of CrossMAb technology for the generation of bi- and multispecific antibodies.” MAbs. Vol. 8. No. 6. Taylor & Francis, 2016, which is incorporated by reference in its entirety. In some embodiments, the CH1 in the heavy chain and the CL domain in the light chain are swapped.

The antigen-binding protein construct can be a Duobody. The Fab-exchange mechanism naturally occurring in IgG4 antibodies is mimicked in a controlled matter in IgG1 antibodies, a mechanism called controlled Fab exchange. This format can ensure specific pairing between the heavy-light chains.

In Dual-variable-domain antibody (DVD-Ig), additional VH and variable light chain (VL) domain are added to each N-terminus for bispecific targeting. This format resembles the IgG-scFv, but the added binding domains are bound individually to their respective N-termini instead of a scFv to each heavy chain N-terminus.

In scFv-IgG, the two scFv are connected to the C-terminus of the heavy chain (CH3). The scFv-IgG format has two different bivalent binding sites and is consequently also called tetravalent. There are no heavy-chain and light-chain pairing problem in the scFv-IgG.

In some embodiments, the antigen-binding protein construct can be have a IgG-IgG format. Two intact IgG antibodies are conjugated by chemically linking the C-terminals of the heavy chains.

The antigen-binding protein construct can also have a Fab-scFv-Fc format. In Fab-scFv-Fc format, a light chain, heavy chain and a third chain containing the Fc region and the scFv are assembled. It can ensure efficient manufacturing and purification.

In some embodiments, antigen-binding protein construct can be a TF. Three Fab fragments are linked by disulfide bridges. Two fragments target the tumor associated antigen (TAA) and one fragment targets a hapten. The TF format does not have an Fc region.

ADAPTIR has two scFvs bound to each sides of an Fc region. It abandons the intact IgG as a basis for its construct, but conserves the Fc region to extend the half-life and facilitate purification.

Bispecific T cell Engager (“BiTE”) consists of two scFvs, VLA VHA and VHB VLB on one peptide chain. It has only binding domains, no Fc region.

In BiTE-Fc, an Fc region is fused to the BiTE construct. The addition of Fc region enhances half-life leading to longer effective concentrations, avoiding continuous IV.

Dual affinity retargeting (DART) has two peptide chains connecting the opposite fragments, thus VLA with VHB and VLB with VHA, and a sulfur bond at their C-termini fusing them together. In DART, the sulfur bond can improve stability over BiTEs.

In DART-Fc, an Fc region is attached to the DART structure. It can be generated by assembling three chains, two via a disulfide bond, as with the DART. One chain contains half of the Fc region which will dimerize with the third chain, only expressing the Fc region. The addition of Fc region enhances half-life leading to longer effective concentrations, avoiding continuous IV.

In tetravalent DART, four peptide chains are assembled. Basically, two DART molecules are created with half an Fc region and will dimerize. This format has bivalent binding to both targets, thus it is a tetravalent molecule.

Tandem diabody (TandAb) comprises two diabodies. Each diabody consists of an VHA and VLB fragment and a VHA and VLB fragment that are covalently associated. The two diabodies are linked with a peptide chain. It can improve stability over the diabody consisting of two scFvs. It has two bivalent binding sites.

The ScFv-scFv-toxin includes toxin and two scFv with a stabilizing linker. It can be used for specific delivery of payload.

In modular scFv-scFv-scFv, one scFv directed against the TAA is tagged with a short recognizable peptide is assembled to a bsAb consisting of two scFvs, one directed against CD3 and one against the recognizable peptide.

In ImmTAC, a stabilized and soluble T cell receptor is fused to a scFv recognizing CD3. By using a TCR, the ImmTAC is suitable to target processed, e.g. intracellular, proteins.

Tri-specific nanobody has two single variable domains (nanobodies) with an additional module for half-life extension. The extra module is added to enhance half-life.

In Trispecific Killer Engager (TriKE), two scFvs are connected via polypeptide linkers incorporating human IL-15. The linker to IL-15 is added to increase survival and proliferation of NKs.

In some embodiments, the antigen-binding protein construct is a bispecific antibody. In some embodiments, the bispecific antibody in present disclosure is designed to be 1+1 (monovalent for each target) and has an IgG1 subtype structure. This can reduce the avidity to cells with low expression levels of HER2 and TROP2, and increase the avidity to cells that co-express HER2 and TROP2, to achieve enhanced targeting function. Mutations S239D and/or I332E (SI mutations) can also be introduced in antibody heavy chains to enhance the antibody affinity to FcγRIIIA.

In some embodiments, the antibodies, the antigen-binding fragments thereof, the antigen-binding protein constructs (e.g., the anti-TROP2 antibody, the anti-HER2 antibody, or the bispecific antibody), or the related antibody drug conjugates (ADC), have a light chain constant region that is at least 80%, 85%, 86%, 87%, 88%, 89%, 90%, 91%, 92%, 93%, 94%, 95%, 96%, 97%, 98%, 99% or 100% identical to SEQ ID NO: 80, and a heavy chain constant region that is at least 80%, 85%, 86%, 87%, 88%, 89%, 90%, 91%, 92%, 93%, 94%, 95%, 96%, 97%, 98%, 99% or 100% identical to SEQ ID NO: 81 or SEQ ID NO: 82.

In some embodiments, the bispecific antibody or antigen-binding fragment thereof described herein has a common light chain.

### Antibody Drug Conjugates (ADC)

The antibodies, the antigen-binding fragments thereof, or the antigen-binding protein constructs (e.g., bispecific antibodies) described herein can be conjugated to a therapeutic agent (a drug). The therapeutic agent can be covalently or non-covalently bind to the antibody or antigen-binding fragment or the antigen binding protein construct (e.g., a bispecific antibody). In some embodiments, the bispecific antibody is an anti-HER2/TROP2 bispecific antibody. In some embodiments, the bispecific antibody has a common light chain.

In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). Useful classes of cytotoxic, cytostatic, or immunomodulatory agents include, for example, antitubulin agents, DNA minor groove binders, DNA replication inhibitors, and alkylating agents.

In some embodiments, the therapeutic agent can include, but not limited to, cytotoxic reagents, such as chemo-therapeutic agents, immunotherapeutic agents and the like, antiviral agents or antimicrobial agents. In some embodiments, the therapeutic agent to be conjugated can be selected from, but not limited to, MMAE (monomethyl auristatin E), MMAD (monomethyl auristatin D), or MMAF (monomethyl auristatin F).

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

In some embodiments, the antigen-binding construct is coupled to the drug via a cleavable linker e.g. a SPBD linker or a maleimidocaproyl-valine-citrulline-p-aminobenzyloxycarbonyl (VC) linker. In some embodiments, the antigen-binding construct is coupled to the drug via a non-cleavable linker e.g. a MCC linker formed using SMCC or sulfo-SMCC. Selection of an appropriate linker for a given ADC can be readily made by the skilled person having knowledge of the art and taking into account relevant factors, such as the site of attachment to the antigen binding construct, any structural constraints of the drug and the hydrophobicity of the drug (see, for example, review in Nolting, Chapter 5, Antibody-Drug Conjugates: Methods in Molecular Biology, 2013, Ducry (Ed.), Springer). A number of specific linker-toxin combinations have been described and may be used with the antigen binding constructs described herein to prepare ADCs in certain embodiments. Examples include, but are not limited to, cleavable peptide-based linkers with auristatins such as MMAE and MMAF, camptothecins such as SN-38, duocarmycins and PBD dimers; non-cleavable MC-based linkers with auristatins MMAF and MMAE; acid-labile hydrazone-based linkers with calicheamicins and doxorubicin; disulfide-based linkers with maytansinoids such as DM1 and DM4, and bis-maleimido-trioxyethylene glycol (BMPEO)-based linkers with maytansinoid DM1. Some these therapeutic agents and linkers are described, e.g., in Peters & Brown, (2015) Biosci. Rep. e00225; Dosio et al., (2014) Recent Patents on Anti-Cancer Drug Discovery 9:35-65; US Patent Publication No. US 2015/0374847, and US20180193477A1; which are incorporated herein by reference in the entirety.

Depending on the desired drug and selected linker, those skilled in the art can select suitable method for coupling them together. For example, some conventional coupling methods, such as amine coupling methods, can be used to form the desired drug-linker complex which still contains reactive groups for conjugating to the antibodies through covalent linkage. In some embodiments, a drug-maleimide complex (i.e., maleimide linking drug) can be used for the payload bearing reactive group in the present disclosure. Most common reactive group capable of bonding to thiol group in ADC preparation is maleimide. Additionally, organic bromides, iodides also are frequently used.

The ADC can be prepared by one of several routes known in the art, employing organic chemistry reactions, conditions, and reagents known to those skilled in the art (see, for example, Bioconjugate Techniques (G. T. Hermanson, 2013, Academic Press). For example, conjugation can be achieved by (1) reaction of a nucleophilic group or an electrophilic group of an antibody with a bivalent linker reagent, to form antibody-linker intermediate Ab-L, via a covalent bond, followed by reaction with an activated drug moiety D; or (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) can be employed with a variety of antibodies, drug moieties, and linkers to prepare the ADCs described here. Various prepared linkers, linker components and toxins are commercially available or may be prepared using standard synthetic organic chemistry techniques. These methods are described e.g., in March's Advanced Organic Chemistry (Smith & March 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press); US20210379193A1, and US20180193477A1, which are incorporated herein by reference in the entirety. In addition, a number of pre-formed drug-linkers suitable for reaction with a selected antigen binding construct are also available commercially, for example, linker-toxins comprising DM1, DM4, MMAE, MMAF or Duocarmycin SA are available from Creative BioLabs (Shirley, N.Y.).

Several specific examples of methods of preparing ADCs are known in the art and are described in U.S. Pat. No. 8,624,003 (pot method), U.S. Pat. No. 8,163,888 (one-step), and U.S. Pat. No. 5,208,020 (two-step method), and US20180193477A1, which are incorporated herein by reference in the entirety. Other methods are known in the art and include those described in Antibody-Drug Conjugates: Methods in Molecular Biology, 2013, Ducry (Ed.), Springer.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, higher drug loading, e.g. p≥5, may cause aggregation, insolubility, toxicity, or loss of cellular permeability of certain antibody-drug conjugates. In certain embodiments, the average drug loading for an antibody-drug conjugate ranges from 1 to about 8; from about 2 to about 6; or from about 3 to about 5. Indeed, it has been shown that for certain antibody-drug conjugates, the optimal ratio of drug moieties per antibody can be around 4. In some embodiments, the DAR is about or at least 1, 2, 3, 4, 5, 6, 7, or 8. In some embodiments, the average DAR in the composition is about 1˜ about 2, about 2˜ about 3, about 3˜ about 4, about 4˜ about 5, about 5˜ about 6, about 6˜ about 7, or about 7˜ about 8.

### Antibody and ADC Characteristics

The anti-HER2 antigen-binding protein construct (e.g., antibodies, bispecific antibodies, or antibody fragments thereof) or ADC derived therefrom can include an antigen-binding region that is derived from any anti-HER2 antibody or any antigen-binding fragment thereof as described herein.

General techniques can be used to measure the affinity of an antibody for an antigen include, e.g., ELISA, RIA, and surface plasmon resonance (SPR). Affinities can be deduced from the quotient of the kinetic rate constants (KD=koff/ka). In some implementations, the antibodies, the antigen-binding fragments thereof, or the antigen-binding protein construct (e.g., bispecific antibody), can bind to ERBB2 (e.g., human HER2, dog ERBB2, monkey ERBB2, and/or mouse ERBB2) with a dissociation rate (koff) of less than 0.1 s⁻¹, less than 0.01 s⁻¹, less than 0.001 s⁻¹, less than 0.0001 s⁻¹, or less than 0.0001 s⁻¹. In some embodiments, the dissociation rate (koff) is greater than 0.01 s⁻¹, greater than 0.001 s⁻¹, greater than 0.0001 s⁻¹, greater than 0.0001 s⁻¹, or greater than 0.00001 s⁻¹. In some embodiments, the dissociation rate (koff) is less than 7×10⁻⁴ s⁻¹.

In some embodiments, kinetic association rates (ka) is greater than 1×10²/Ms, greater than 1×10³/Ms, greater than 1×10⁴/Ms, greater than 1×10⁵/Ms, or greater than 1×10⁶/Ms. In some embodiments, kinetic association rates (ka) is less than 1×10⁵/Ms, less than 1×10⁶/Ms, or less than 1×10²/Ms. In some embodiments, kinetic association rates (ka) is greater than 1.3×10⁵/Ms.

In some embodiments, the antibodies, the antigen-binding fragments thereof, or the antigen-binding protein construct (e.g., bispecific antibody) can bind to ERBB2 (e.g., human HER2, dog ERBB2, monkey ERBB2, and/or mouse ERBB2) with a KD of less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 5 nM, 4 nM, 3 nM, 2 nM, or 1 nM. In some embodiments, KD is greater than 1×10⁻⁷M, greater than 1×10⁻⁸ M, greater than 1×10⁻⁹ M, or greater than 1×10⁻¹⁰ M. In some embodiments, the antibody binds to human HER2 with KD less than or equal to about 5 nM, 4.5 nM, 4 nM, 3 nM or 0.27 nM.

The anti-TROP2 antigen-binding protein construct (e.g., bispecific antibodies) or ADC derived therefrom can also include an antigen-binding region that is derived from any anti-TROP2 antibody or antigen-binding fragment thereof as described herein.

In some implementations, the antibodies, the antigen-binding fragments thereof, or the antigen-binding protein constructs (e.g., bispecific antibody) can bind to TROP2 (e.g., human TROP2, dog TROP2, monkey TROP2, and/or mouse TROP2) with a dissociation rate (koff) of less than 0.1 s⁻¹, less than 0.01 s⁻¹, less than 0.001 s⁻¹, less than 0.0001 s⁻¹, or less than 0.0001 s⁻¹. In some embodiments, the dissociation rate (koff) is greater than 0.01 s⁻¹, greater than 0.001 s⁻¹, greater than 0.0001 s⁻¹, greater than 0.0001 s⁻¹, or greater than 0.00001 s⁻¹.

In some embodiments, kinetic association rates (ka) is greater than 1×10²/Ms, greater than 1×10³/Ms, greater than 1×10⁴/Ms, greater than 1×10⁵/Ms, or greater than 1×10⁶/Ms. In some embodiments, kinetic association rates (ka) is less than 1×10⁵/Ms, less than 1×10⁶/Ms, or less than 1×10⁷/Ms.

Affinities can be deduced from the quotient of the kinetic rate constants (KD=koff/ka). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M. In some embodiments, the antibody binds to human TROP2 with KD less than or equal to about 15 nM or 10 nM.

Because the antigen-binding protein construct (e.g., bispecific antibody) binds to both TROP2 and HER2, for cells that express both TROP2 and HER2, the antigen-binding protein construct has a higher binding affinity to these cells. Avidity can be used to measure the binding affinity of an antigen-binding protein construct to these cells. Avidity is the accumulated strength of multiple affinities of individual non-covalent binding interactions.

Thermal stabilities can also be determined. The antibodies, the antigen-binding fragments thereof, or the antigen-binding protein constructs (e.g., bispecific antibody), or the ADC derived therefrom as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. As IgG can be described as a multi-domain protein, the melting curve sometimes shows two transitions, with a first denaturation temperature, Tm D1, and a second denaturation temperature Tm D2. The presence of these two peaks often indicate the denaturation of the Fc domains (Tm D1) and Fab domains (Tm D2), respectively. When there are two peaks, Tm usually refers to Tm D2. Thus, in some embodiments, the antibodies or antigen binding fragments as described herein has a Tm D1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm D2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm, Tm D1, Tm D2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies, the antigen-binding fragments thereof, the antigen-binding protein constructs (e.g., the anti-TROP2 antibody, the anti-HER2 antibody, or the bispecific antibody), or the ADC derived therefrom, have an endocytosis rate in cells (e.g., in NCI-N87 cells or NCI-H292 cells) that is at least 50%, 60%, 70%, 72.5%, 75%, 77.5%, 80%, 82.5%, 85%, 87.5%, 90%, 91%, 92%, 93%, 94%, 95%, 96%, 97%, 98%, or 99%. In some embodiments, the endocytosis rate that is less than 50%, 60%, 70%, 72.5%, 75%, 77.5%, 80%, 82.5%, 85%, 87.5%, 90%, 91%, 92%, 93%, 94%, 95%, 96%, 97%, 98%, or 99%.

In some embodiments, the antibodies, the antigen-binding fragments thereof, the antigen-binding protein constructs (e.g., bispecific antibody), or the ADC derived therefrom, can bind to dog ERRB2, monkey ERRB2, or mouse ERRB2. In some embodiments, the binding is measured by the percentage of positive cells as determined by FACS. In some embodiments, the percentage of positive cells is greater than 50%, 60%, 70%, 72.5%, 75%, 77.5%, 80%, 82.5%, 85%, 87.5%, 90%, 91%, 92%, 93%, 94%, 95%, 96%, 97%, 98%, or 99%. In some embodiments, the percentage of positive cells is less than 50%, 60%, 70%, 72.5%, 75%, 77.5%, 80%, 82.5%, 85%, 87.5%, 90%, 91%, 92%, 93%, 94%, 95%, 96%, 97%, 98%, or 99%. In some embodiments, the antibodies, the antigen-binding fragments thereof, the antigen-binding protein constructs (e.g., bispecific antibody), or the ADC derived therefrom, cannot bind to dog ERRB2, monkey ERRB2, or mouse ERRB2.

In some embodiments, the antibodies, the antigen-binding fragments thereof, the antigen-binding protein constructs (e.g., bispecific antibody), or the ADC derived therefrom, can bind to dog TROP2, monkey TROP2, or mouse TROP2. In some embodiments, the binding is measured by the percentage of positive cells as determined by FACS. In some embodiments, the percentage of positive cells is greater than 50%, 60%, 70%, 72.5%, 75%, 77.5%, 80%, 82.5%, 85%, 87.5%, 90%, 91%, 92%, 93%, 94%, 95%, 96%, 97%, 98%, or 99%. In some embodiments, the percentage of positive cells is less than 50%, 60%, 70%, 72.5%, 75%, 77.5%, 80%, 82.5%, 85%, 87.5%, 90%, 91%, 92%, 93%, 94%, 95%, 96%, 97%, 98%, or 99%. In some embodiments, the antibodies, the antigen-binding fragments thereof, the antigen-binding protein constructs (e.g., bispecific antibody), or the ADC derived therefrom, cannot bind to dog TROP2, monkey TROP2, or mouse TROP2.

In some embodiments, the antibodies, the antigen-binding fragments thereof, the antigen-binding protein constructs (e.g., the anti-TROP2 antibody, the anti-HER2 antibody, or the bispecific antibody), or the ADC derived therefrom, has a purity that is greater than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by HPLC. In some embodiments, the antibodies, the purity is less than 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by HPLC.

In some embodiments, the antibodies, the antigen-binding fragments thereof, the antigen-binding protein constructs (e.g., the anti-TROP2 antibody, the anti-HER2 antibody, or the bispecific antibody), or or the ADC derived therefrom, has a yield that is greater than 20, 30, 40, 50, 60, 70, 80, 90, 100, 200, 300, 400, 500, 600, or 700 (μg/mL). In some embodiments, the yield is less than 20, 30, 40, 50, 60, 70, 80, 90, 100, 200, 300, 400, 500, 600, or 700 (μg/mL).

In some embodiments, the stability of the antibodies, the antigen-binding fragments thereof, the antigen-binding protein constructs (e.g., the anti-TROP2 antibody, the anti-HER2 antibody, or the bispecific antibody), or the ADC derived therefrom, is measured by the Capillary Isoelectric Focusing (cIEF) method (indicated as the percentages of the main component, acidic component, and alkaline component). In some embodiments, the percentage of the main component is greater than 10%, 20%, 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, after being subject to various conditions e.g., as measured by cIEF. In some embodiments, the condition is storing at 4° C. for at least or about 1, 2, 3, 4, 5, 6, 7, 8, 9, or 10 days, storing at 25° C. for at least or about 1, 2, 3, 4, 5, 6, 7, 8, 9, or 10 days, or storing at 40° C. for at least or about 1, 2, 3, 4, 5, 6, 7, 8, 9, or 10 days. In some embodiments, the condition is freeze-thaw for at least or about 1, 2, 3, 4, 5, 6, 7, 8, 9, 10, 11, 12, 13, 14, 15, 16, 17, 18, 19, 20, 30, or 50 times. In some embodiments, the condition is storing the composition at pH3.5 for about or at least 0, 1, 2, 3, 4, 5, 6, 7, 8, 9, or 10 hours. In some embodiments, after the treatment, the percentage of the acidic component is greater than 10%, 20%, 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by cIEF. In some embodiments, after the treatment, the percentage of the alkaline component is greater than 10%, 20%, 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by cIEF. In some embodiments, after the treatment, the percentage of the main component is less than 10%, 20%, 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by cIEF. In some embodiments, the percentage of the acidic component is less than 10%, 20%, 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by cIEF. In some embodiments, the percentage of the alkaline component is less than 10%, 20%, 30%, 40%, 50%, 60%, 70%, 72.5%, 75%, 77.5%, 80%, 82.5%, 85%, 87.5%, 90%, 91%, 92%, 93%, 94%, 95%, 96%, 97%, 98%, or 99%, e.g., as measured by cIEF.

In some embodiments, the antibodies, the cell killing ability of the antigen-binding fragments thereof, the antigen-binding protein constructs (e.g., the anti-TROP2 antibody, the anti-HER2 antibody, or the bispecific antibody), or the ADC derived therefrom, is measured by IC50 (ng/ml) (e.g., in NCI-N87 or NCI-H292 cells). In some embodiments, the IC50 is greater than 2, 5, 10, 20, 30, 40, 50, 100, 200, 500, 1000, 2000, 5000, 10000, 20000, 50000, 100000, 200000, or 500000 ng/mL. In some embodiments, the IC50 is less than 2, 5, 10, 20, 30, 40, 50, 100, 200, 500, 1000, 2000, 5000, 10000, 20000, 50000, 100000, 200000, or 500000 ng/mL.

In some embodiments, the antibodies, the antigen-binding fragments thereof, the antigen-binding protein constructs (e.g., the anti-TROP2 antibody, the anti-HER2 antibody, or the bispecific antibody), or the ADC derived therefrom, has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( Ti - T 0 ) / ( Vi - V 0 ) ] \times 100$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibody, the antigen-binding fragment thereof, or the antigen-binding protein construct (e.g., bispecific antibody) has a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4. In some embodiments, one or both mutations S239D and/or I332E (SI mutations) are introduced in antibody Fc region to enhance the antibody affinity to FcγRIIIA, thereby increasing ADCC effects. A detailed description of SI mutations can be found in U.S. Pat. No. 7,662,925, which is incorporated by reference in their entirety.

In some embodiments, the antibody, the antigen-binding fragment thereof, or the antigen-binding protein construct (e.g., bispecific antibody) does not have a functional Fc region. For example, the antibodies or antigen binding fragments are Fab, Fab′, F(ab′)2, and Fv fragments.

In some embodiments, the antibody, the antigen-binding fragment thereof, or the antigen-binding protein construct (e.g., bispecific antibody) is incorporated in an antibody drug conjugate.

### Recombinant Vectors

The present disclosure also provides recombinant vectors (e.g., expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153:516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

The disclosure provides one or more nucleic acid encoding any of the polypeptides as described herein. In some embodiments, the nucleic acid (e.g., cDNA) includes a polynucleotide encoding a polypeptide of a heavy chain as described herein. In some embodiments, the nucleic acid includes a polynucleotide encoding a polypeptide of a light chain as described herein. In some embodiments, the nucleic acid includes a polynucleotide encoding a scFv polypeptide as described herein.

In some embodiments, the vector can have two of the nucleic acids as described herein, wherein the vector encodes the VL region and the VH region that together bind to HER2. In some embodiments, a pair of vectors is provided, wherein each vector comprises one of the nucleic acids as described herein, wherein together the pair of vectors encodes the VL region and the VH region that together bind to HER2. In some embodiments, the vector includes two of the nucleic acids as described herein, wherein the vector encodes the VL region and the VH region that together bind to TROP2. In some embodiments, a pair of vectors is provided, wherein each vector comprises one of the nucleic acids as described herein, wherein together the pair of vectors encodes the VL region and the VH region that together bind to TROP2. In some embodiments, the VL regions are identical.

Vectors can also be constructed to express specific antibodies or polypeptides. In some embodiments, a vector can be constructed to co-express anti-HER2 antibody light chain (HER2-K) and heavy chain (HER2-H). In some embodiments, a vector can contain sequences of, from 5′ end to 3′ end, cytomegalovirus promotor (CMV), HER2-K, polyadenylation (PolyA), CMV, HER2-H, PolyA, simian vacuolating virus 40 terminator (SV40) and glutamine synthetase marker (GS). In some embodiments, a vector can be constructed to co-express anti-TROP2 antibody light chain (TROP2-K) and anti-TROP2 antibody heavy chain (TROP2-H). In some embodiments, a vector can contain sequences of, from 5′ end to 3′ end, CMV, HER2-K, PolyA, CMV, HER2-H, SV40 and GS. In some embodiments, a vector can be constructed to express anti-TROP2 antibody scFv polypeptide chain.

### Methods of Making Antibodies, Antigen Binding Fragments and Antigen Binding Protein Constructs

An isolated fragment of human protein can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Phage display (panning) can be used to optimize antibody sequences with desired binding affinities. In this technique, a gene encoding single chain Fv (comprising VH or VL) can be inserted into a phage coat protein gene, causing the phage to “display” the scFv on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding affinities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

In some embodiments, a covalent modification can be made to the antibodies, the antigen-binding fragments thereof, or the antigen-binding protein constructs (e.g., bispecific antibodies). These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, knob-into-hole (KIH) technology can be used, which involves engineering CH3 domains to create either a “knob” or a “hole” in each heavy chain to promote heterodimerization. The KIH technique is described e.g., in Xu, Yiren, et al. “Production of bispecific antibodies in ‘knobs-into-holes’ using a cell-free expression system.” MAbs. Vol. 7. No. 1. Taylor & Francis, 2015, which is incorporated by reference in its entirety. In some embodiments, one heavy chain has a T366W, and/or S354C (knob) substitution (EU numbering), and the other heavy chain has an Y349C, T366S, L368A, and/or Y407V (hole) substitution (EU numbering). In some embodiments, one heavy chain has one or more of the following substitutions Y349C and T366W (EU numbering). The other heavy chain can have one or more the following substitutions E356C, T366S, L368A, and Y407V (EU numbering). Furthermore, a substitution (-ppcpScp-->-ppcpPcp-) can also be introduced at the hinge regions of both substituted IgG.

Furthermore, an anion-exchange chromatography can be used to purify bispecific antibodies. Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion. Anion exchange chromatography can be used to purify proteins based on their isoelectric point (pI). The isoelectric point is defined as the pH at which a protein has no net charge. When the pH>pI, a protein has a net negative charge and when the pH<pI, a protein has a net positive charge. Thus, in some embodiments, different amino acid substitution can be introduced into two heavy chains, so that the pI for the homodimer comprising two Arm A and the pI for the homodimer comprising two Arm B is different. The pI for the bispecific antibody having Arm A and Arm B will be somewhere between the two pIs of the homodimers. Thus, the two homodimers and the bispecific antibody can be released at different pH conditions. The present disclosure shows that a few amino acid residue substitutions can be introduced to the heavy chains to adjust pI.

Bispecific antibodies can also include e.g., cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

### Methods of Treatment

The methods described herein include methods for the treatment of disorders associated with cancer. Generally, the methods include administering a therapeutically effective amount of engineered antibodies, the antigen-binding fragments thereof, the antigen-binding protein constructs (e.g., bispecific antibodies), or the antibody drug conjugates as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the esophageal, cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. In some embodiments, the cancer is a chemotherapy resistant cancer.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of antibodies, the antigen-binding fragments thereof, the antigen-binding protein constructs (e.g., bispecific antibodies), or an antibody drug conjugate described herein to a subject in need thereof, e.g., a subject having, or identified or diagnosed as having, a cancer, e.g., breast cancer, carcinoid, cervical cancer, colorectal cancer, endometrial cancer, glioma, head and neck cancer, liver cancer, lung cancer, lymphoma, melanoma, ovarian cancer, pancreatic cancer, prostate cancer, renal cancer, skin cancer, stomach cancer, esophageal carcinoma, testis cancer, thyroid cancer, or urothelial cancer.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals. In some embodiments, the subject is a human. In some embodiments, the subject is a dog.

In some embodiments, the cancer is thyroid cancer, urothelial cancer, breast cancer, colorectal cancer, renal cancer, cervical cancer, ovarian cancer, lung cancer, endometrial cancer, skin cancer, stomach cancer, esophageal carcinoma, pancreatic cancer, prostate cancer, liver cancer, lymphoma, or glioma.

In some embodiments, the cancer is cervical cancer, prostate cancer, thyroid cancer, urothelial cancer, head and neck cancer, endometrial cancer, ovarian cancer, lung cancer, breast cancer, carcinoid, skin cancer, liver cancer, or testis cancer.

In some embodiments, the cancer is pancreas cancer, lung cancer, stomach cancer, prostate cancer, breast cancer, ovary cancer, colon cancer, skin cancer, or brain cancer.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, or an antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment, or antibody-drug conjugate may vary, depending on, inter alia, patient history as well as other factors such as the type (and/or dosage) of the composition used.

Effective amounts and schedules for administering the antibodies, antibody-encoding polynucleotides, antibody-drug conjugates, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, antibody-drug conjugates, and/or compositions disclosed herein, the route of administration, the particular type of antibodies, antibody-encoding polynucleotides, antigen binding fragments, antibody-drug conjugates, and/or compositions disclosed herein used and other drugs being administered to the mammal.

A typical daily dosage of an effective amount of an antibody, the antigen-binding fragment thereof, the antigen-binding protein construct (e.g., a bispecific antibody) or the antibody drug conjugate is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about or at least 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg. 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, the antigen-binding fragment thereof, or the antigen-binding protein construct (e.g., a bispecific antibody), antibody-drug conjugates, or pharmaceutical composition (e.g., any of the antibodies, antigen-binding fragments, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody, the antigen-binding fragment thereof, the antigen-binding protein construct (e.g., a bispecific antibody), or antibody-drug conjugate, and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, antibody-drug conjugate, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, antibody-drug conjugate, or pharmaceutical composition (e.g., any of the antibodies, antigen-binding antibody fragments, antibody-drug conjugate, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) or antibody-drug conjugate in the subject.

In some embodiments, the subject can be administered the at least one antibody, antigen-binding antibody fragment, antibody-drug conjugate, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments, antibody-drug conjugates (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAMI agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD-1 antibody, an anti-PD-L1 antibody, an anti-LAG-3 antibody, an anti-TIGIT antibody, an anti-BTLA antibody, or an anti-GITR antibody.

### Pharmaceutical Compositions and Routes of Administration

Also provided herein are pharmaceutical compositions that contain at least one (e.g., one, two, three, or four) of the antigen-binding protein constructs, antibodies (e.g., bispecific antibodies), antigen-binding fragments, or antibody-drug conjugates described herein. Two or more (e.g., two, three, or four) of any of the antigen-binding protein constructs, antibodies, antigen-binding fragments, or antibody-drug conjugates described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antigen-binding protein constructs, antibodies, antigen-binding fragments, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Exemplary doses include milligram or microgram amounts of any of the antigen-binding protein constructs, antibodies or antigen-binding fragments, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 0.1 mg/kg to about 0.5 mg/kg).

The pharmaceutical compositions can be included in a container, pack, or dispenser together with instructions for administration. The disclosure also provides methods of manufacturing the antibodies or antigen binding fragments thereof, or antibody-drug conjugates for various uses as described herein.

### EXAMPLES

The invention is further described in the following examples, which do not limit the scope of the invention described in the claims.

### Example 1. Detection of HER2 and TROP2 Expression

Functional genomic RNA-seq was performed to determine the expression level of HER2 and TROP2 in different human tumor cell lines. The results are shown in the following table.

TABLE 1
RNA seq results (FPKM screening threshold: 15)

| | | Tissue | | | |
|---|---|---|---|---|---|
| NO. | Cell Name | types | Source | HER2 | TROP2 |
| 1 | Capan-1 | Pancreas | ATCC, Cat #: HTB-79 | 48 | 163 |
| 2 | Panc 02.03 | Pancreas | ATCC, Cat #: CRL-2553 | 44 | 1236 |
| 3 | NCI-H1781 | Lung | ATCC, Cat #: CRL-5894 | 88 | 370 |
| 4 | NCI-H1650 | Lung | ATCC, Cat #: CRL-5883 | 32 | 1100 |
| 5 | HCC827 | Lung | ATCC, Cat #: CRL-2868 | 28 | 368 |
| 6 | NCI-N87 | Stomach | ATCC, Cat #: CRL-5822 | 1444 | 761 |
| 7 | NUGC-4 | Stomach | Cobioer, Cat #: | 61 | 72 |
| | | | CBP60493 | | |
| 8 | DU 145 | Prostate | ATCC, Cat #: HTB-81 | 17 | 162 |
| 9 | BT_20 | Breast | ATCC, Cat #: HTB-19 | 27 | 221 |
| 10 | ZR-75-1 | Breast | ATCC, Cat #: CRL-1500 | 69 | 70 |
| 11 | SK-BR-3 | Breast | ATCC, Cat #: HTB-30 | 811 | 336 |
| 12 | HCC70 | Breast | ATCC, Cat #: CRL-2315 | 20 | 1187 |
| 13 | BT-474 | Breast | ATCC, Cat #: HTB-20 | 519 | 22 |
| 14 | HCC1954 | Breast | ATCC, Cat #: CRL-2338 | 1005 | 1057 |
| 15 | OVCAR-3 | Ovary | ATCC, Cat #: HTB-161 | 15 | 150 |
| 16 | SK-OV-3 | Ovary | ATCC, Cat #: HTB-77 | 331 | 145 |
| 17 | DLD-1 | Colon | ATCC, Cat #: CCL-221 | 19 | 137 |
| 18 | COLO 205 | Colon | ATCC, Cat #: CCL-222 | 33 | 52 |
| 19 | COLO 201 | Colon | ATCC, Cat #: CCL-224 | 47 | 102 |
| 20 | HCT-8 | Colon | ATCC, Cat #: CCL-244 | 26 | 35 |
| 21 | A-431 | Skin | ATCC, Cat #: CRL-1555 | 18 | 1797 |

FPKM: Reads per kilo base per million mapped reads

Furthermore, flow cytometry (FACS) was used to detect the expression of HER2 and TROP2 proteins in the following human tumor cells. Labeled antibodies including anti-hTROP2-PE (R&D Systems, Inc., Cat #: FAB650P) and PE anti-human CD340 (erbB2/HER-2) (Biolegend Inc., Cat #: 324406) were used. The geometric mean fluorescence intensity (MFI) results are shown in the table below.

TABLE 2
HER2 and TROP expression results in different cell lines

| | | Tissue | | | |
|---|---|---|---|---|---|
| NO. | Cell line | types | Source | HER2 | TROP2 |
| 1 | NCI-H1650 | Lung | ATCC, Cat #: CRL-5883 | 25741 | 123249 |
| 2 | HCC827 | Lung | ATCC, Cat #: CRL-2868 | 11094 | 82675 |
| 3 | NCI-N87 | Stomach | ATCC, Cat #: CRL-5822 | 392949 | 14348 |
| 4 | DU 145 | Prostate | ATCC, Cat #: HTB-81 | 9788 | 19741 |
| 5 | ZR-75-1 | Breast | ATCC, Cat #: CRL-1500 | 45502 | 43987 |
| 6 | BT-474 | Breast | ATCC, Cat #: HTB-20 | 309012 | 17643 |
| 7 | HCC1954 | Breast | ATCC, Cat #: CRL-2338 | 229418 | 161383 |
| 8 | A-431 | Skin | ATCC, Cat #: CRL-1555 | 15353 | 280176 |
| 9 | NCI-H1975 | Lung | ATCC, Cat #: CRL-5908 | 14768 | 12442 |
| 10 | MDA- | Breast | ATCC, Cat #: HTB-26 | 6803 | 2477 |
| | MB-23 | | | | |
| 11 | NCI-H292 | Lung | ATCC, Cat #: CRL-1848 | 11963 | 224754 |
| 12 | U-87 MG | Brain | ATCC, Cat #: HTB-14 | 584 | 373 |
| 13 | SNU-5 | Stomach | ATCC, Cat #: CRL-5973 | 15301 | 533 |
| 14 | Hs 746T | Stomach | ATCC, Cat #: HTB-135 | 161 | 172 |
| 15 | MDA- | Breast | Shanghai Institutes for | 617 | 13546 |
| | MB-46 | | Biological Sciences | | |
| 16 | BxPC-3 | Pancreas | ATCC, Cat #: CRL-1687 | 7968 | 14495 |
| 17 | A549 | Lung | ATCC, Cat #: CCL-185 | 8542 | 183 |
| 18 | MCF7 | Breast | ATCC, Cat #: HTB-22 | 6814 | 57151 |
| 19 | LoVo | Colon | ATCC, Cat #: CCL-229 | 6455 | 166 |

The data showed that HER2 and TROP2 were highly expressed in a variety of tumor cell lines.

### Example 2. Anti-HER2 Antibody

### Generating Anti-HER2 Antibody

Human HER2 protein (ACRO Biosystems Inc., Cat #: HE2-H5253, including positions 23-652 of SEQ ID NO: 64 of human HER2 protein) or DNA encoding this protein was emulsified with adjuvants, and was used to immunize RenLite™ mice (Biocytogen, complete human heavy chain variable domain combined with a common light chain substitution in situ). The mice are described e.g., in PCT/CN2021/097652, which is incorporated herein by reference in its entirety. Before immunization, retro-orbital blood was collected as a negative control.

Freund's complete adjuvant CFA was used for the first immunization and Freund's incomplete adjuvant IFA was used for the second, third, and fourth immunizations. A total of four immunizations were performed. The first and second immunizations were separated by two weeks, and the remaining immunizations were separated by one week. One week after the fourth immunization, retro-orbital blood was collected, and the antibody titer of serum was detected by ELISA. One week later, mice with high titer were further injected with human HER2 Protein through the tail vein for impulse immunization.

Antigen-specific immune cells were isolated from the immunized mice to further obtain anti-HER2 antibodies or to obtain the light chain and heavy chain variable region sequences of the anti-HER2 antibodies. For example, single cell technology (for example, using Beacon® Optofluidic System, Berkeley Lights Inc.) was used to screen and find plasma cells that secrete antigen-specific monoclonal antibodies, and reverse transcription and PCR sequencing were used to obtain antibody variable region sequences. The obtained variable region sequences were used for antibody expression to verify the binding affinity to HER2 using FACS. Exemplary antibodies obtained by this method included: H-2A10, H-1H2, H-2B2, H-3E5, H-3C6, and H-3C8. These antibodies contained substantially the same light chain, and their VL and VL CDR 1-3 sequences are shown in FIGS. 8 & 9. The VH and VL regions of H-1H2, H-2B2, H-3E5, H-3C6 and H-3C8 are shown in FIG. 10.

Various IgG1, IgG2 and IgG4 antibodies were made. With respect to the name of the antibodies, when the antibody VH\VL is connected to different isotypes, the isotype is added to the name. For example, if the VH and VL of H-1H2 are connected to IgG1 constant regions, the antibody is named as H-1H2-IgG1. Examples of other isotypes are as follows: H-1H2-IgG2, H-1H2-IgG4.

### Anti-HER2 Antibody Endocytosis Test

Sample anti-HER2 antibodies (2.5 μg/mL) and pHAb-Goat Anti-Human IgG Secondary Antibody (Promega (Beijing) Biotech Co., Ltd., Cat #: G9845) were added to BT-474 cells with a high HER2 expression. After incubating for 1 hour, the cells were centrifuged and washed with FACS buffer. The MFI values were measured with a flow cytometer, and the endocytosis rates of the anti-HER2 antibodies were calculated. The results are shown in the table below (Table 3). For isotype control, an antibody targeting an irrelevant target protein was used.

TABLE 3
Anti-HER2 Antibody Endocytosis Rates

| Antibody | MFI | Percentage of Positive (%) |
|---|---|---|
| Isotype control | 9841 | 1.1% |
| Trastuzumab analog | 50377 | 88.0% |
| Pertuzumab analog | 100376 | 97.1% |
| H-1H2-IgG1 | 364980 | 99.9% |
| H-2B2-IgG1 | 282143 | 99.9% |
| H-3E5-IgG1 | 290833 | 99.8% |
| H-3C6-IgG1 | 324006 | 99.9% |
| H-3C8-IgG1 | 269321 | 99.9% |
| H-2A10-IgG1 | 115617 | 99.8% |

Trastuzumab is a HER2 targeting humanized monoclonal antibody, and its heavy chain and light chain sequences are shown in SEQ ID NOs: 65-66.

Pertuzumab is also a HER2 targeting antibody, and its heavy chain and light chain sequences are shown in SEQ ID NOs: 67-68.

The data showed that H-1H2-IgG1, H-2B2-IgG1, H-3E5-IgG1, H-3C6-IgG1 and H-3C8-IgG1 had good cell endocytosis rates compared with isotype control (ISO), positive control Trastuzumab and Pertuzumab.

### Anti-HER2 Antibody Cross-Species Binding Analyses

BT-474 cells expressing human HER2 (hHER2), CHO-S cells (CHO-fasHER2) expressing monkey HER2 (fasHER2), and NIH3T3 cells (NIH3T3-dHER2) expressing Canis lupus familiaris HER2 (dHER2) were transferred to a 96-well plate at a density of 5×10⁴ cells/well. Serially diluted sample anti-HER2 antibodies were added to the 96-well plate, and incubated at 4° C. for 30 min. Then, the cells were incubated with the secondary antibody Anti-hIgG-Fc-Alex Flour 647 (RL1-H) (Jackson ImmunoResearch Laboratories, Inc., 109-606-170) at 4° C. in the dark for 15 minutes before flow cytometry analysis.

CHO-fasHER2 cells were obtained by transfecting CHO-S cells with positions 174-804 of Macaca fascicularis (crab-eating macaque) HER2 amino acid sequence (SEQ ID NO: 74). NIH3T3-dHER2 cells were obtained by transfecting NIH3T3 (ATCC, Cat #: CRL1658) cells with a construct containing the amino acid sequence of Canis lupus familiaris (dog) HER2 (SEQ ID NO: 75) (the construct contains CAG Promoter-dog HER2 CDS-P2A-EGFP-WPRE-PA).

The test results are shown in the table below. All five anti-HER2 antibodies can bind to hHER2 and fasHER2. Among these five antibodies, H-1H2-IgG1, H-2B2-IgG1 and H-3C8-IgG1 also showed cross-species binding with hHER2, fasHER2 and dHER2.

TABLE 4
Anti-HER2 antibody cross-species binding analyses

| Antibody | HER2 Protein | Percentage of Positive Cells | Evaluation |
|---|---|---|---|
| H-1H2-IgG1 | hHER2 | 100% | Binding |
| | fasHER2 | 46.6% | Binding |
| | dHER2 | 79.6% | Binding |
| H-2B2-IgG1 | hHER2 | 99.9% | Binding |
| | fasHER2 | 42.3% | Binding |
| | dHER2 | 90.3% | Binding |
| H-3E5-IgG1 | hHER2 | 100% | Binding |
| | fasHER2 | 43.4% | Binding |
| | dHER2 | 1.87% | No binding |
| H-3C6-IgG1 | hHER2 | 99.9% | Binding |
| | fasHER2 | 67.1% | Binding |
| | dHER2 | 2.66% | No binding |
| H-3C8-IgG1 | hHER2 | 100% | Binding |
| | fasHER2 | 48.1% | Binding |
| | dHER2 | 95.6% | Binding |

### Anti-HER2 Antibody Affinity Test

The affinity of the anti-HER2 antibodies to His-tagged HER2 protein of human (hHER2, ACROBiosystems Inc., Cat #: HE2-H5225) (positions 23-652 of SEQ ID NO: 64), mouse (mHER2, ACROBiosystems Inc., Cat #: ER2-M5220) (SEQ ID NO: 76), dog (dHER2, Sino Biological Inc., Cat #: 70024-D08H) (positions 1-652 of SEQ ID NO: 75) or monkey (fasHER2, ACROBiosystems Inc., Cat #: HE2-C52Hb) (SEQ ID NO: 74) were measured by Biolayer Interferometry (BLI) using ForteBio Octet system at 30° C. A total of 5 monoclonal antibodies were tested: H-1H2-IgG1, H-2B2-IgG1, H-3E5-IgG1, H-3C6-IgG1 and H-3C8-IgG1.

Anti-HER2 antibodies were loaded onto AHC biosensor (ForteBio Inc., Cat #: 18-5060) at 10 μg/mL to yield a response of 1.0 nm. Kinetic measurements were performed at the concentrations 12.5 nM, 25 nM, 50 nM, 100 nM, 200 nM, and 400 nM of the recombinant His-tagged HER2 protein. The association phase lasted for 180 s and the dissociation phase lasted 300 s followed by a regeneration step with 10 mM Glycine-HCl, pH1.7. Data analysis was performed using the Octet data analysis program (DataAnalysis 11) using a standard 1:1 binding model. Affinity values were deduced from the quotient of the kinetic rate constants (KD=koff/kon).

As a person of ordinary skill in the art would understand, the same method with appropriate adjustments for parameters (e.g., antibody concentration) was performed for each tested anti-HER2 antibody. The results for the tested antibodies are summarized in the table below.

TABLE 5
Affinity test results

| Antibody | HER2 protein | kon (1/Ms) | koff (1/s) | KD (M) |
|---|---|---|---|---|
| H-1H2-IgG1 | hHER2 | 4.19E+05 | 1.65E−02 | 3.93E−08 |
| | fasHER2 | 4.16E+05 | 1.67E−02 | 4.00E−08 |
| | mHER2 | No binding | — | — |
| | dHER2 | 4.71E+05 | 1.50E−03 | 3.19E−09 |
| H-2B2-IgG1 | hHER2 | 2.33E+05 | 1.05E−02 | 4.49E−08 |
| | fasHER2 | 2.51E+05 | 9.59E−03 | 3.82E−08 |
| | mHER2 | No binding | — | — |
| | dHER2 | 3.49E+05 | 1.52E−02 | 4.36E−08 |
| H-3E5-IgG1 | hHER2 | 1.56E+05 | 6.56E−04 | 4.20E−09 |
| | fasHER2 | 1.24E+05 | 9.07E−04 | 7.33E−09 |
| | mHER2 | No binding | — | — |
| | dHER2 | No binding | — | — |
| H-3C6-IgG1 | hHER2 | 1.50E+05 | 2.32E−04 | 1.55E−09 |
| | fasHER2 | 1.63E+05 | 2.94E−04 | 1.80E−09 |
| | mHER2 | No binding | — | — |
| | dHER2 | No binding | — | — |
| H-3C8-IgG1 | hHER2 | 4.56E+05 | 2.36E−03 | 5.19E−09 |
| | fasHER2 | 4.60E+05 | 2.38E−03 | 5.16E−09 |
| | mHER2 | No binding | — | — |
| | dHER2 | 4.78E+05 | 2.92E−03 | 6.11E−09 |

The results showed that all five anti-HER2 antibodies showed good binding affinity to human HER2 and monkey HER2. Among these five antibodies, H-1H2-IgG1, H-2B2-IgG1, H-3C8-IgG1 also showed good binding affinity to dog HER2.

### Example 3. Preparation and Analysis of Anti-TROP2 Antibodies

### Generating Anti-TROP2 Antibody

hTrop2-Fc (ACROBiosystems Inc., Cat #: TR2-H5253, including human TROP2 protein SEQ ID NO: 69 amino acids 31-274) and mTrop2-his (ACROBiosystems Inc., Cat #: TR2-M52H6, including amino acids 25-270 of Mus musculus TROP2 protein SEQ ID NO: 79) were used to cross-immunize RenLite™ mice. Before immunization, retro-orbital blood was collected from the mice as a negative control. Freund's complete adjuvant CFA was used for the first immunization, and Freund's incomplete adjuvant IFA was used for the second, third, and fourth immunizations. A total of four immunizations were conducted. One week after the third immunization, retro-orbital blood was collected, and the serum titer of anti-human TROP2 antibodies and anti-mouse TROP2 antibodies were detected by ELISA. One week later, the mice with a high titer were selected for the fourth immunization by subcutaneous injection of mTrop2-his. Two weeks after the fourth immunization, CHO-S cells expressing human TROP2 antigen were used for impulse immunization by tail vein injection and hTROP2-Fc was used for impulse immunization by intraperitoneal injection.

Antigen-specific immune cells were isolated (from immune organs of immunized mice) to further obtain anti-TROP2 antibodies or to obtain the light chain and heavy chain V region sequences of the anti-TROP2 antibodies. Single-cell technology (such as Beacon® Optofluidic System, Berkeley Lights Inc.) was used to screen and find plasma cells secreting antigen-specific monoclonal antibodies, and then reverse transcription and PCR sequencing were used to obtain the antibody variable region sequences. The variable region sequences were cloned into a skeleton vector containing human IgG constant region for antibody expression to verify the binding of the expressed antibodies to TROP2 by FACS. Exemplary fully human antibodies obtained by this method included: T-3A4, T-4B9, T-4C12, T-5C8, and T-6F7. These antibodies have the same light chain, and the sequence is consistent with the common light chain sequence of the anti-HER2 antibody. The CDR sequences and VH and VL sequences of T-3A4, T-4B9 and T-6F7 are shown in FIGS. 11-13.

Various IgG1, IgG2, and IgG4 antibodies were made. The constant region can also include some mutations. For example, when SI mutations (EU numbering: S239D and I332E mutations) are introduced into the Fc region of T-6F7-IgG1, the resulting antibody is named as T-6F7-IgG1-SI.

### Anti-TROP2 Antibody Endocytosis Detection

Anti-TROP2 antibodies (1.25 μg/mL) and pHAb-Goat Anti-Human IgG Secondary Antibody were added to the CHO cells (CHO-hTROP2) that highly express human TROP2 (SEQ ID NO: 69). After incubating for 1 h, the cells were centrifuged and washed in FACS buffer. MFI was detected on a flow cytometer, and the endocytosis rates of anti-TROP2 antibodies were calculated. The results are shown in the table below.

TABLE 6
Anti-TROP2 Antibody Endocytosis Rates

| | | Percentage |
|---|---|---|
| Antibody | MFI | of Positive (%) |
| Sacituzumab govitecan antibody analog | 22965 | 51.2% |
| DS-1062 antibody analog | 21847 | 43.9% |
| T-3A4-IgG1-SI | 23169 | 69.0% |
| T-4B9-IgG1-SI | 20844 | 62.7% |
| T-4C12-IgG1-SI | 23929 | 71.9% |
| T-5C8-IgG1-SI | 29914 | 82.9% |
| T-6F7-IgG1-SI | 29478 | 92.2% |

Sacituzumab govitecan (Trodelvy™) is a humanized anti-TROP2 monoclonal antibody-drug conjugate. The heavy chain and light chain sequences of Sacituzumab govitecan analog are shown as SEQ ID NO: 70-71.

DS-1062 is a TROP2-directed antibody drug conjugate, which is in phase III clinical trials in patients with advanced or metastatic non-small cell lung cancer (NSCLC). The heavy chain and light chain sequences of DS-1062 are shown as SEQ ID NO: 72-73.

The antibody portion of Sacituzumab govitecan and DS-1062 were used for comparison purpose. The data showed that compared with the control (Sacituzumab govitecan antibody analog and DS-1062 antibody analog), T-3A4-IgG1, T-4B9-IgG1, T-4C12-IgG1, T-5C8-IgG1 and T-6F7-IgG1 all have good endocytosis rates.

### Anti-TROP2 Cross-Species Binding Analyses

CHO-hTROP, CHO-S cells expressing Macaca fascicularis (crab-eating macaque) TROP2 (fasTROP2, SEQ ID NO: 77) (CHO-fasTROP2), and CHO-S cells expressing Canis lupus familiaris (dog) TROP2 (dTROP2, SEQ ID NO: 78) (CHO-dTROP2) were transferred to a 96-well plate at a density of 5×10⁴ cells/well. Gradient dilutions of the sample antibodies were added into the 96-well plate, and incubated at 4° C. for 30 min. After that, the cells were incubated with Anti-hIgG-Fc-Alex Flour 647 (RL1-H) (Jackson ImmunoResearch Laboratories, Inc., Cat #: 109-606-170) at 4° C. in the dark for 15 minutes before flow cytometry analysis. The results are shown in the following table:

TABLE 7
Anti-TROP2 antibody cross-species binding analyses

| | TROP2 | Percentage of | |
|---|---|---|---|
| Antibody | protein | Positive Cells | Evaluation |
| T-3A4-IgG1-SI | hTROP2 | 57.1% | Binding |
| | fasTROP2 | 82.8% | Binding |
| | dTROP2 | 37.3% | Binding |
| T-4B9-IgG1-SI | hTROP2 | 72.6% | Binding |
| | fasTROP2 | 73.4% | Binding |
| | dTROP2 | 68.3% | Binding |
| T-4C12-IgG1-SI | hTROP2 | 64.0% | Binding |
| | fasTROP2 | 67.4% | Binding |
| | dTROP2 | 70.6% | Binding |
| T-5C8-IgG1-SI | hTROP2 | 63.9% | Binding |
| | fasTROP2 | 75.7% | Binding |
| | dTROP2 | 23.1% | Binding |
| T-6F7-IgG1-SI | hTROP2 | 65.0% | Binding |
| | fasTROP2 | 70.9% | Binding |
| | dTROP2 | 39.0% | Binding |

The results showed that T-6F7-IgG1-SI, T-4C12-IgG1-SI, T-3A4-IgG1-SI, T-5C8-IgG1-SI and T-4B9-IgG1-SI all can bind to human TROP2, monkey TROP2, and dog TROP2.

### Anti-TROP2 Antibody Affinity Test

The affinity of the anti-TROP2 antibodies to His-tagged TROP2 protein of human (hTROP2, ACROBiosystems Inc., Cat #: TR2-H5223), Canine (dTROP2, ACROBiosystems Inc., Cat #: TR2-C52H4) and Rhesus macaque/Cynomolgus (fasTROP2, ACROBiosystems Inc., Cat #: R52H3) were measured by surface plasmon resonance (SPR) using Biacore (Biacore, INC, Piscataway N.J.) 8K biosensor equipped with pre-immobilized Protein A sensor chips. Purified anti-TROP2 antibodies were diluted to 0.5 μg/mL and then injected into the Biacore 8K biosensor at 10 L/min for about 50 seconds to achieve a desired protein density (e.g., about 120 response units (RU)). His-tagged TROP2 protein at concentrations of 200 nM was then injected at 30 μL/min for 180 seconds. Dissociation was monitored for 600 seconds. The chip was regenerated after the last injection of each titration with Glycine (pH 2.0, 30 μL/min for 30 seconds).

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below.

TABLE 8
Anti-TROP2 antibody affinity test results

| | TROP2 | | | | Eval- |
|---|---|---|---|---|---|
| Antibody | protein | kon (1/Ms) | koff (1/s) | KD (M) | uation |
| T-3A4-IgG1-SI | hTROP2 | 9.70E+04 | 4.58E−03 | 4.72E−08 | Binding |
| | fasTROP2 | 5.98E+04 | 2.55E−03 | 4.27E−08 | Binding |
| | dTROP2 | 8.62E+03 | 1.89E−04 | 2.19E−08 | Binding |
| T-4B9-IgG1-SI | hTROP2 | 5.68E+05 | 5.82E−03 | 1.02E−08 | Binding |
| | fasTROP2 | 7.81E+05 | 5.56E−03 | 7.12E−09 | Binding |
| | dTROP2 | 4.34E+06 | 5.75E−02 | 1.32E−08 | Binding |
| T-4C12- | hTROP2 | 7.65E+04 | 7.34E−03 | 9.59E−08 | Binding |
| IgG1-SI | fasTROP2 | 5.84E+04 | 8.65E−03 | 1.48E−07 | Binding |
| | dTROP2 | 9.35E+05 | 2.75E−03 | 2.93E−09 | Binding |
| T-5C8-IgG1-SI | hTROP2 | 6.40E+04 | 3.44E−03 | 5.38E−08 | Binding |
| | fasTROP2 | 7.18E+04 | 1.59E−03 | 2.21E−08 | Binding |
| | dTROP2 | Not | — | — | Not |
| | | Binding | | | Binding |
| T-6F7-IgG1-SI | hTROP2 | 1.65E+05 | 4.28E−03 | 2.60E−08 | Binding |
| | fasTROP2 | 1.17E+05 | 2.21E−03 | 1.90E−08 | Binding |
| | dTROP2 | 4.71E+04 | 9.23E−05 | 1.96E−09 | Binding |

The results show that all tested antibodies have good binding affinity to human TROP2. Among the tested antibodies, T-3A4-IgG1-SI, T-4B9-IgG1-SI and T-6F7-IgG1-SI also have cross-species binding affinity to monkey and dog TROP2.

### Example 4. Anti-HER2/TROP2 Bispecific Antibody

### Preparation of Anti-HER2/TROP2 Bispecific Antibody

Anti-HER2 antibodies (H-1H2, H-2B2, H-3E5, H-3C6 and H-3C8) and anti-TROP2 antibodies (T-3A4, T-4B9, T-4C12, T-5C8 and T-6F7) can be paired to form various bispecific antibodies. Vectors for the light chain and heavy chain of the antibodies were made. Three vectors were co-transfected into CHO-S cells. After 14 days of culture, the cell supernatant was collected and purified by Protein A affinity chromatography.

Various methods can be used to reduce the chance of wrong pairing between the two heavy chains. In the Fc region, knobs-into-holes mutations were introduced to the anti-TROP2 arm heavy chain and the anti-HER2 arm heavy chain. Exemplary bispecific antibodies obtained included: H-1H2-T-6F7, H-2B2-T-6F7, H-3C8-T-6F7 and T-6F7-H-1H2. To verify the binding affinity of bispecific antibodies, anti-HER2 or anti-TROP control bispecific antibodies were also constructed, where one arm of the control bispecific antibody recognizes HER2 or TROP, and the other arm recognizes CD28 (CD28 RenLite co-light chain antibody). These control bispecific antibodies were named as H-2B2-CD28, H-3C8-CD28, CD28-H-1H2, and CD28-T-6F7.

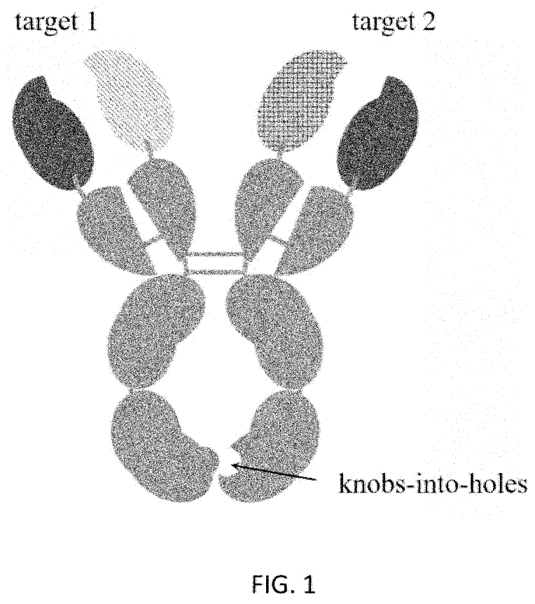

These antibodies all have the knobs-into-holes mutations. In H-1H2-T-6F7, the heavy chain constant region of H-1H2 has knob mutations, and the constant region of T-6F7 has hole mutations. In T-6F7-H-1H2, the heavy chain constant regions of T-6F7 has knob mutations, and the heavy chain constant regions of H-1H2 has hole mutations. An exemplary antibody structure is shown in FIG. 1, where target 1 and target 2 can be HER2 and TROP2 respectively, or TROP2 and HER2 respectively, or HER2 and CD28 respectively, or CD28 and HER2 respectively, or CD28 and TROP2, respectively.

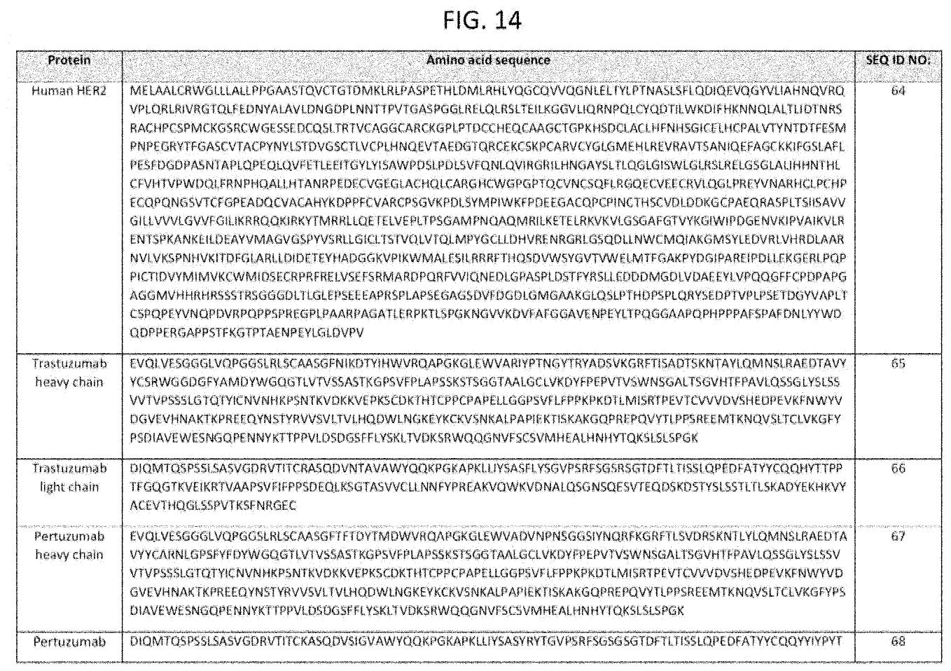

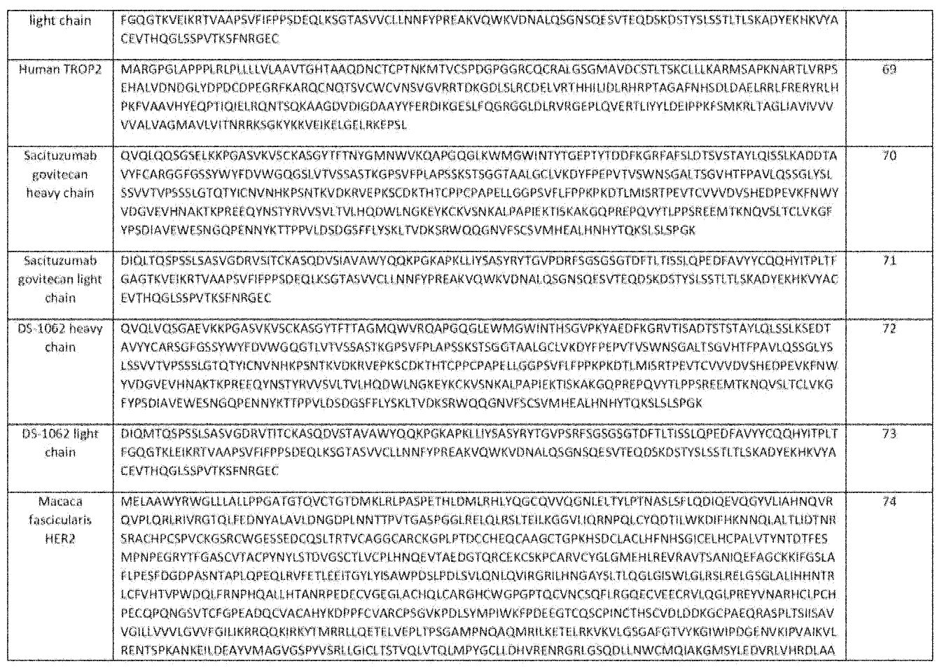

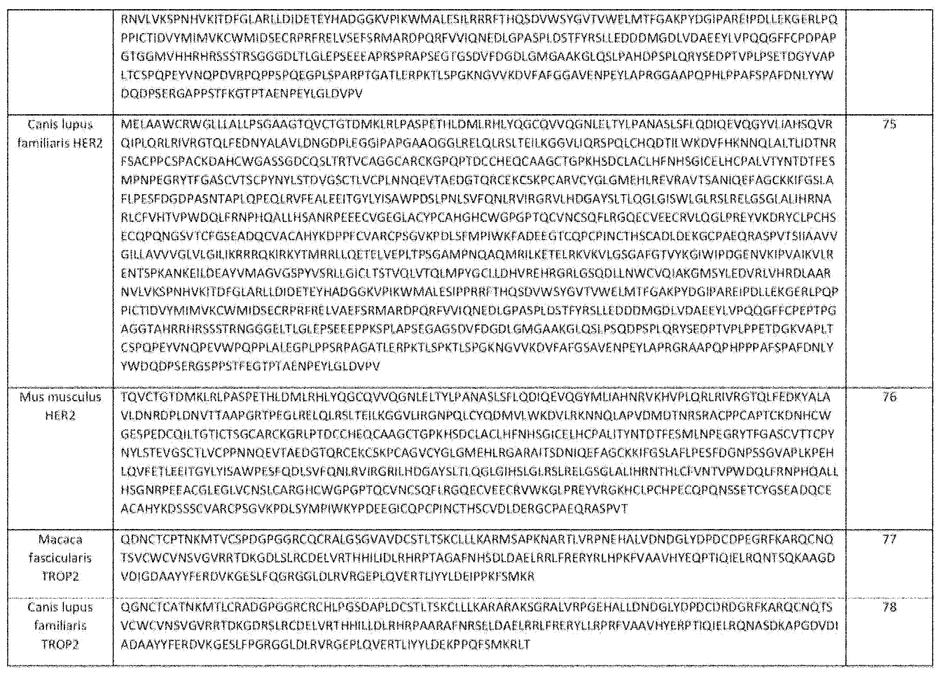

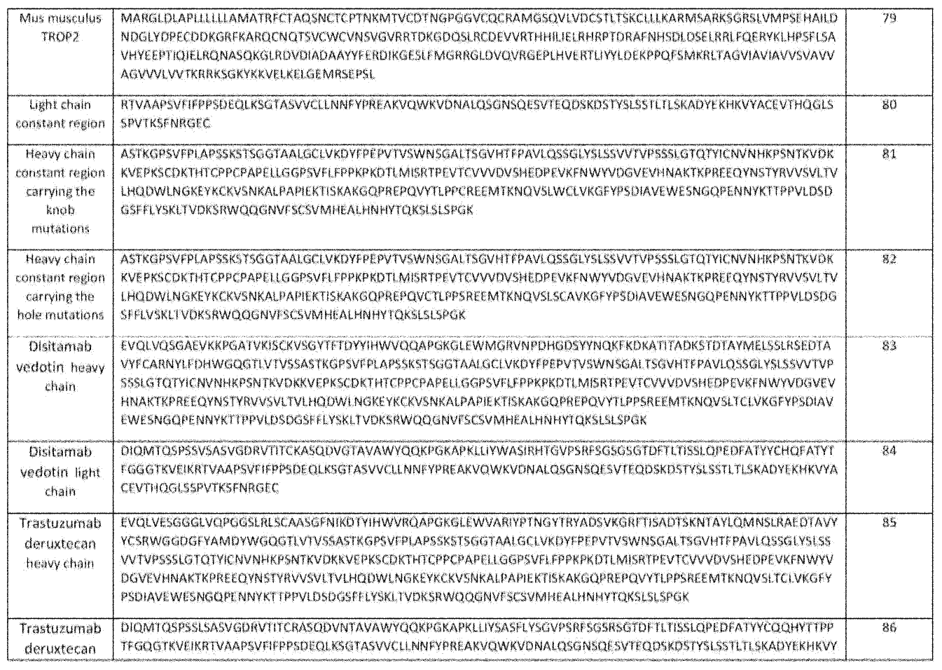

The sequences of the light chain constant regions, the heavy chain constant region carrying the knob mutations, and the heavy chain constant region carrying the hole mutations are shown in FIG. 14.

Purified anti-HER2/TROP2 bispecific antibodies were analyzed by a non-reducing SDS-PAGE (sodium dodecyl sulphate-polyacrylamide gel electrophoresis) and SEC-HPLC (size exclusion chromatography-high performance liquid chromatography).

Non-reducing SDS-PAGE was performed using a 4-12% acrylamide gel. The protein samples were prepared as follows. First, 2.4 μL of the protein sample was mixed with 6 μL Tris-Glycine SDS Sample Buffer (2×) (Invitrogen LC2676) and 3.6 μL distilled water. The mixture was then boiled for 2 minutes and instantly centrifuged before loading. 4 μg of each sample was loaded to the gel.

In the SEC-HPLC method, the antibody samples were diluted to 1 mg/mL with purified water and an Agilent 1290 chromatograph system (connected with XBridge™ Protein BEH SEC column (200 Å, Waters Corporation)) was used. The following parameters were used: mobile phase: 25 mmol/L phosphate buffer (PB)+300 mmol/L NaCl, pH 6.8; flow rate: 1.8 mL/min; column temperature: 25° C.; detection wavelength: 280 nm; injection volume: 10 μL; sample tray temperature: about 4° C.; and running time: 7 minutes. Results are summarized in the table below.

TABLE 9
Preparation of anti-HER2/TROP2 bispecific antibody

| ID | Sample | Yield (μg/mL) | SEC-HPLC |
|---|---|---|---|
| 1 | H-2B2-T-6F7 | 431.15 | 90.01% |
| 2 | H-3C8-T-6F7 | 517.96 | 91.30% |
| 3 | H-1H2-T-6F7 | 716.54 | 90.80% |
| 4 | H-1H2-T-4B9 | 188.31 | 60.07% |
| 5 | H-2B2-T-4B9 | 48.00 | 38.31% |
| 6 | H-3C8-T-4B9 | 20.73 | 36.77% |
| 7 | H-1H2-T-3A4 | 255.00 | 85.53% |
| 8 | H-2B2-T-3A4 | 331.46 | 60.12% |
| 9 | H-3C8-T-3A4 | 567.69 | 62.87% |
| 10 | T-6F7-H-1H2 | 657.69 | 96.67% |

### Anti-HER2/TROP2 Bispecific Antibody Binding Activity Verification

The binding activity of anti-HER2/TROP2 antibodies to NCI-H292 cells and NCI-N87 cells was verified by flow cytometry. The secondary antibody used in the experiment was: AF647-conjugated Goat Anti-human IgG (Jackson ImmunoResearch Laboratories, Inc., Cat #: 109-606-170). Results are summarized in the table below.

TABLE 10
Combined flow cytometry test results

| Antibody | NCI-H292 | NCI-N87 |
|---|---|---|
| NC | 0.78% | 1.72% |
| NC+ secondary antibody | 1.21% | 2.97% |
| Trastuzumab analog | 97.8% | 98.5% |
| Sacituzumab analog | 97.4% | 98.7% |
| H-2B2-T-6F7 | 97.1% | 98.9% |
| T-6F7-H-1H2 | 97.6% | 98.7% |

The results show that H-2B2-T-6F7 and T-6F7-H-1H2 can both bind to NCI-H292 cells and NCI-N87 cells that simultaneously express HER2 and TROP2.

### Validation of Antibody Binding to Canine Tumor Cell Lines

The binding activity of anti-HER2 antibody, anti-TROP antibody and anti-HER2/TROP2 antibody to the canine breast cancer cell line CMT-U27 (ATCC, Cat #: CRL-3456) was measured by flow cytometry. The secondary antibody used in the experiment is FITC-conjugated Goat Anti-human IgG (Jackson ImmunoResearch Laboratories, Inc., Cat #: 109-096-170). Results are summarized in the table below.

TABLE 11
Combined flow cytometry test results

| Antibody | CMT-U27 |
|---|---|
| NC | 0.74% |
| NC+ secondary antibody | 0.82% |
| Human IgG1 Control | 1.59% |
| H-1H2-IgG1 | 97.6% |
| H-2B2-IgG1 | 98.0% |
| H-3C8-IgG1 | 97.8% |
| T-6F7-IgG1-SI | 1.74% |
| T-6F7-H-1H2 | 82.9% |
| H-2B2-T-6F7 | 96.8% |
| H-3C8-T-6F7 | 98.3% |

The results showed that anti-HER2 monoclonal antibodies H-1H2-IgG1, H-2B2-IgG1 and H-3C8-IgG1, and anti-HER2/TROP2 bispecific antibodies T-6F7-H-1H2, H-2B2-T-6F7 and H-3C8-T-6F7 can all bind to the canine cell line CMT-U27.

### Anti-HER2/TROP2 Bispecific Antibody Endocytosis Test

Anti-HER2 antibodies, anti-TROP2 antibodies, or anti-HER2/TROP2 bispecific antibodies and/or goat anti-human IgG secondary antibodies were added to NCI-N87 cells that highly express human HER2 and TROP2, respectively, and incubated for 1 hour. The cells were centrifuged and washed with FACS buffer. MFI was measured by a flow cytometer. Endocytosis rates of antibodies to NCI-N87 cells were calculated. The results are shown in the following table (Table 12). For isotype control, an antibody targeting an irrelevant target protein was used.

For direct detection, the primary antibodies (e.g., anti-HER2 antibodies, anti-TROP2 antibodies, or anti-HER2/TROP2 bispecific antibodies) were directly labeled with pH sensitive markers to detect their endocytosis rates. For indirect detection, goat anti-human IgG secondary antibodies labeled with pH sensitive markers were used to detect the endocytosis of the primary antibodies (e.g., anti-HER2 antibodies, anti-TROP2 antibodies, or anti-HER2/TROP2 bispecific antibodies).

TABLE 12
Antibody endocytosis rates

| | Direct detection | | Indirect detection | |
|---|---|---|---|---|
| | (Primary | | (Primary antibody + | |
| | antibody) | | Secondary antibody) | |
| | | Percentage | | Percentage |
| | | of Positive | | of Positive |
| Antibody | MFI | Population | MFI | Population |
| NC | 4778 | 1.39% | 4871 | 1.11% |
| Isotype control | 4899 | 1.86% | 4924 | 1.88% |
| Trastuzumab analog | 18924 | 82.5% | 23420 | 87.3% |
| Pertuzumab analog | 15748 | 74.7% | 20988 | 79.5% |
| Sacituzumab analog | 16622 | 70.1% | 16087 | 72.2% |
| H-2B2-IgG1 | 25903 | 90.8% | 33590 | 91.5% |
| H-3C8-IgG1 | 24575 | 91.1% | 36342 | 92.8% |
| H-1H2-IgG1 | 20192 | 88.2% | 34929 | 92.2% |
| T-6F7-IgG1-SI | 12177 | 45.1% | 33652 | 89.9% |
| H-2B2-CD28 | 7988 | 10.4% | 41603 | 88.2% |
| H-3C8-CD28 | 11483 | 46.0% | 50727 | 93.5% |
| CD28-H-1H2 | 7061 | 6.27% | 58524 | 96.2% |
| CD28-T-6F7 | 5120 | 1.86% | 19808 | 68.4% |
| H-2B2-T-6F7 | 20290 | 82.8% | 54948 | 95.5% |
| H-3C8-T-6F7 | 27439 | 88.8% | 49078 | 93.5% |
| T-6F7-H-1H2 | 17498 | 78.6% | 59289 | 92.8% |
| H-1H2-T-6F7 | 19963 | 86.3% | 57243 | 94.0% |

The results showed the following results regarding the endocytosis rates: H-3C8-T-6F7>H-2B2-T-6F7>T-6F7-H-1H2 in the bispecific antibody. The bispecific antibodies showed similar or higher endocytosis rates than the corresponding monoclonal antibody. Single-arm control bispecific antibodies (H-2B2-CD28, H-3C8-CD28, CD28-H-1H2 and CD28-T-6F7) showed significantly lower endocytosis rates than the corresponding bispecific antibodies or monoclonal antibodies.

For indirect detection, the marker-labeled goat anti-human secondary antibodies may have caused cross-linking of the primary antibodies (e.g., anti-HER2 antibodies, anti-TROP2 antibodies, or anti-HER2/TROP2 bispecific antibodies), leading to enhanced endocytosis.

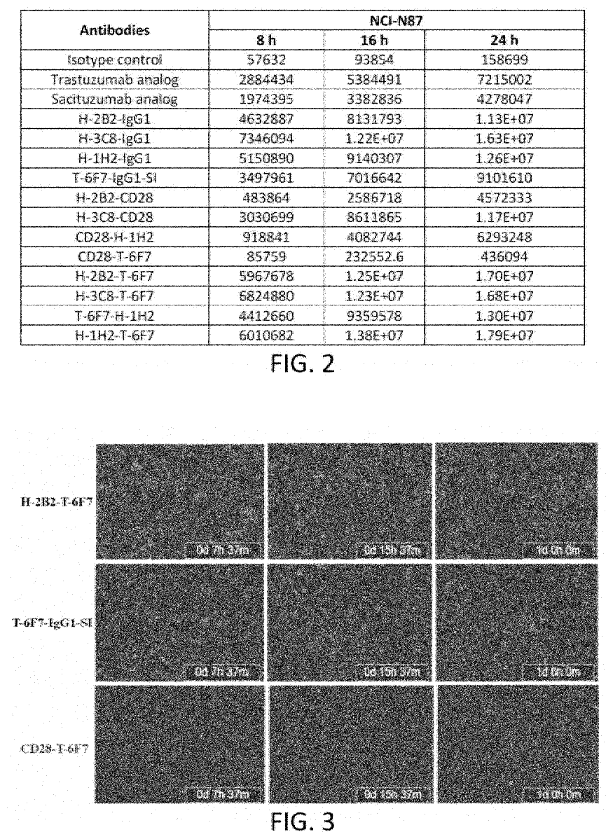

Further, the purified antibodies H-1H2-T-6F7, H-2B2-T-6F7, H-3C8-T-6F7 and T-6F7-H-1H2 were incubated in cell culture with NCI-N87 cells or NCI-H292 cells, respectively. IncuCyte (Sartorius AG, IncuCyte® S3) was incubated for 24 hours to detect the endocytosis of the antibodies. The endocytosis results of NCI-N87 and NCI-H292 cells are shown in FIGS. 2-5.

The endocytosis results in NCI-N87 cells are shown in FIGS. 2 and 3.

In the NCI-N87 cell line, in terms of 24 h endocytosis efficiency, the bispecific antibodies showed similar/higher endocytosis than the corresponding monoclonal antibodies, and the control bispecific antibodies showed significantly reduced endocytosis activities. The endocytosis rates of the bispecific antibodies are as follows: H-3C8-T-6F7>H-2B2-T-6F7>T-6F7-H-1H2.

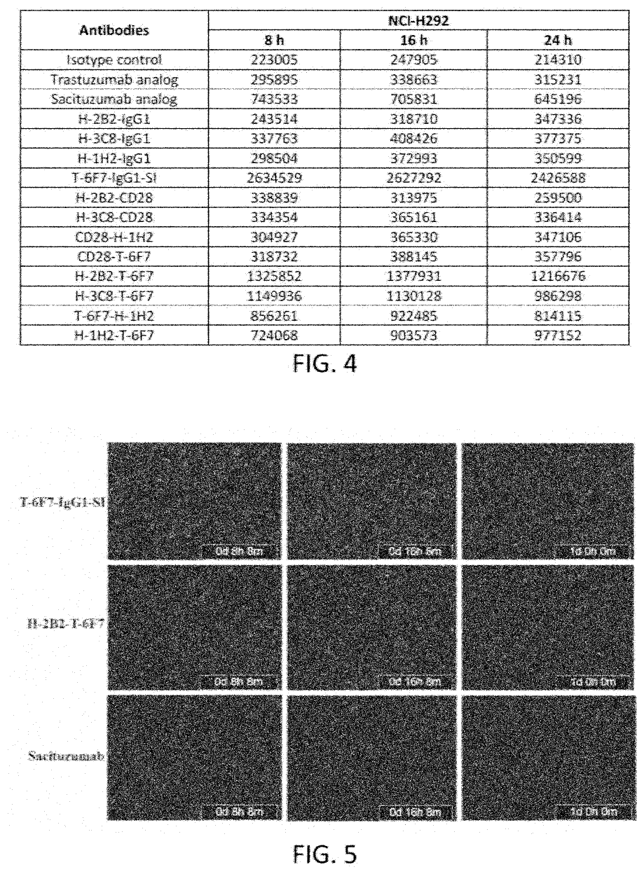

The endocytosis results in NCI-H292 cells are shown in FIGS. 4 and 5.

In the NCI-H292 cell line, the endocytosis rates within 24 h are as follows: The endocytosis efficiency of the bispecific antibody is weaker than that of the T-6F7-IgG1-SI monoclonal antibody, but stronger than that of the anti-HER2 monoclonal antibody and the control bispecific antibodies.

### Stability of Anti-HER2/TROP2 Bispecific Antibodies

Four anti-HER2/TROP2 antibodies H-1H2-T-6F7, H-2B2-T-6F7, T-6F7-H-1H2 and H-3C8-T-6F7 were diluted to 5 mg/mL using a buffer at pH 6.0 (3 mg/mL histidine, 80 mg/mL sucrose, and 0.2 mg/mL Tween 80). The diluted antibodies were kept in sealed Eppendorf tubes at 4±3° C. (hereinafter referred to as 4° C.) for 7 days; or at 40±3° C. (hereinafter referred to as 40° C.) for 7 days, and their thermal stability was evaluated.

Specifically, the following tests were performed: (1) observing the solution appearance and presence of visible non-soluble objects; (2) detecting the purity changes of antibodies by Size-Exclusion High Performance Liquid Chromatography (SEC-HPLC) (indicated as the percentage of the main peak area to the sum of all peak areas (Purity, %)); (3) detecting changes in the apparent hydrophobicity of the antibodies using the Hydrophobic Interaction Chromatography-High Performance Liquid Chromatography (HIC-HPLC) method (indicated as the retention time of the main peak (HIC, min)); (4) detecting the purity changes of antibodies by capillary electrophoresis-sodium dodecyl sulphate (CE-SDS) under reducing (CE-SDS (R)) and non-reducing (CE-SDS (NR)) conditions (indicated as the percentage of the main peak area to the sum of all peak areas (Purity, %)); (5) detecting charge variants in the antibodies by the Capillary Isoelectric Focusing (cIEF) method (indicated as the percentages of the main component, acidic component, and alkaline component).

In the SEC-HPLC experiments, the antibody samples were diluted to 1 mg/mL with purified water and an Agilent 1290 chromatograph system (connected with XBridge™ Protein BEH SEC column (200 Å, Waters Corporation)) was used. The following parameters were used: mobile phase: 25 mmol/L phosphate buffer (PB)+300 mmol/L NaCl, pH 6.8; flow rate: 1.8 mL/min; column temperature: 25° C.; detection wavelength: 280 nm; injection volume: 10 μL; sample tray temperature: about 4° C.; and running time: 7 minutes.

In the HIC-HPLC experiments, an Agilent 1260 chromatograph system (connected with ProPac™ HIC-10 column (4.6×250 mm, Thermo Scientific)) was used, and samples were diluted using mobile phase A to 0.5 mg/mL. The following parameters were used: mobile phase A: 1.0 M ammonium sulfate, 20 mM sodium acetate, 10% acetonitrile pH 6.5; mobile phase B: 20 mM sodium acetate, 10% acetonitrile pH 6.5; flow rate: 0.8 mL/min; gradient: 0 min 100% A, 2 min 100% A, 32 min 100% B, 34 min 100% B, 35 min 100% A, and 45 min 100% A; column temperature: 30° C.; detection wavelength: 280 nm; injection volume: 10 μL; sample tray temperature: about 10° C.; and running time: 30 minutes.

In the cIEF experiments, a Maurice cIEF Method Development Kit (Protein Simple, Cat #: PS-MDK01-C) was used for sample preparation. Specifically, 8 μL protein sample was mixed with the following reagents in the kit: 1 μL Maurice cIEF pI Marker-4.05, 1 μL Maurice cIEF pI Marker-9.99, 35 μL 1% Methyl Cellulose Solution, 2 μL Maurice cIEF 500 mM Arginine, 4 μL Ampholytes (Pharmalyte pH ranges 3-10), and water (added to make a final volume of 100 μL). On the Maurice analyzer (Protein Simple, Santa Clara, CA), Maurice cIEF Cartridges (PS-MC02-C) were used to generate imaging capillary isoelectric focusing spectra. The sample was focused for a total of 10 minutes. The analysis software installed on the instrument was used to integrate the absorbance of the 280 nm-focused protein.

In the CE-SDS experiments, Maurice (Protein simple, Maurice M) and Maurice CE-SDS Size Application Kit (Protein simple, Cat #: PS-MAK02-S) were used.

In CE-SDS (NR), 54 μL Sample Buffer, 6 μL antibody sample, 2.4 μL 25× internal standard, 3 μL 250 nM Iodoacetamide (SIGMA, Cat #: 16125) were add to a microcentrifuge tube, followed by centrifugation at 3000 rpm for 1 min and heating in a 70° C. water bath for 10 min. The samples were then cooled to room temperature followed by centrifugation at 10000 rpm for 3 minute. Supernatant sample preparations were then transferred to a 96 well plate and tested in Maurice. The following parameters were used: injection voltage 4.6 kV, injection time 20 sec, separation voltage 5.75 kV, and separation time 40 min.

In CE-SDS (R), 54 μL Sample Buffer, 6 μL antibody sample, 2.4 μL 25× internal standard, 3 μL 2-Mercaptoethanol (SIGMA, Cat #: M6250) were add to a microcentrifuge tube, followed by centrifugation at 3000 rpm for 1 min and heating in a 70° C. water bath for 10 min. The samples were then cooled to room temperature followed by centrifugation at 10000 rpm for 3 minute. 50 μL supernatant sample preparations were then transferred to a 96 well plate and tested in Maurice. The injection voltage was 4.6 kV, the injection time was 20 sec, the separation voltage was 5.75 kV, and the separation time was 30 min.

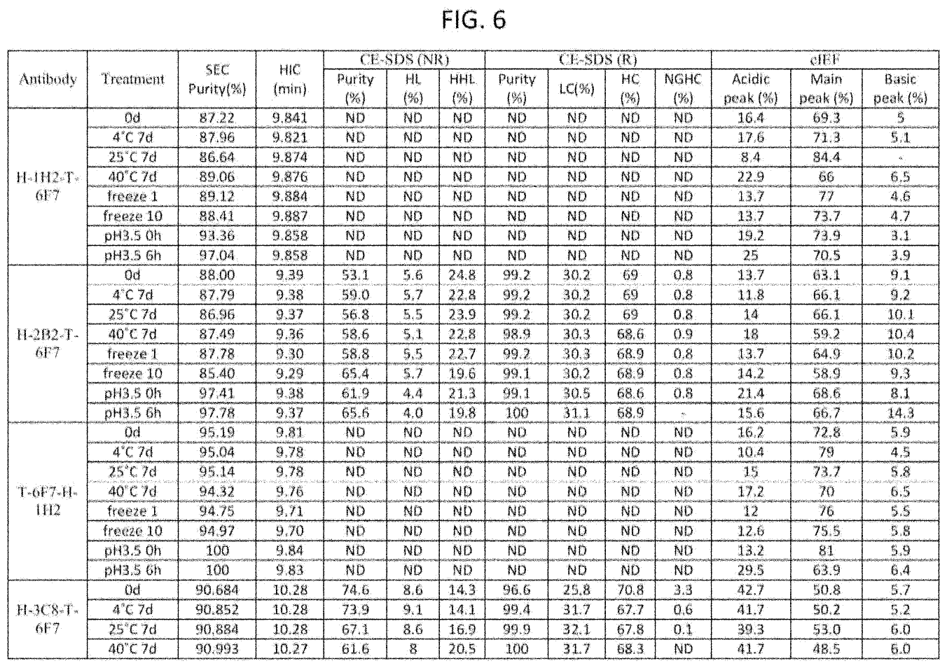

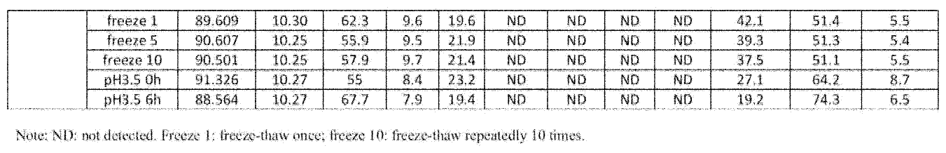

Detailed results of H-1H2-T-6F7, H-2B2-T-6F7, T-6F7-H-1H2 and H-3C8-T-6F7 are shown in FIG. 6.

The results showed that H-3C8-T-6F7-IgG1 and H-2B2-T-6F7-IgG1 have better stability and physical and chemical properties.

### Example 5. Antibody Drug Conjugate

After Protein A purification, H-2B2-IgG1, H-3C8-IgG1, H-1H2-IgG1, T-6F7-IgG1-SI, H-2B2-CD28, H-3C8-CD28, CD28-T-6F7, CD28-H-1H2, H-2B2-T-6F7, H-3C8-T-6F7, H-1H2-T-6F7 and T-6F7-H-1H2 were dialyzed and concentrated into PBS buffer by ultrafiltration. The concentration was determined by UV absorption. These antibodies were used for the subsequent antibody drug coupling reactions.

Coupling of Antibodies with Drug Molecules

The purified antibodies H-2B2-IgG1, H-3C8-IgG1, H-1H2-IgG1, T-6F7-IgG1-SI, H-2B2-CD28, H-3C8-CD28, CD28-T-6F7, CD28-H-1H2, H-2B2-T-6F7, H-3C8-T-6F7, H-1H2-T-6F7 and T-6F7-H-1H2 were coupled with MMAE (monomethyl auristatin E) or MMAF (monomethyl auristatin F) through a maleimidocaproyl-valine-citrulline-p-aminobenzyloxycarbonyl (VC) linker. The drug-antibody ratio (DAR) of the obtained ADC was 3 to 4.

For the names of antibody-drug conjugates, “ADC” is added directly after the antibody name. For example, if H-2B2-IgG1 is coupled to MMAE, it is named as H-2B2-IgG1-ADC.

SEC-HPLC and HIC-HPLC were used to detect the coupling of antibodies with drug molecules. SEC-HPLC test results are shown in the table below (Table 13). For isotype-control-ADC, a human IgG1 isotype control was coupled to MMAE to form isotype-control-ADC.

TABLE 13
SEC chromatography

| | ADC | | | | Antibody | | |
|---|---|---|---|---|---|---|---|
| ADC | HMW | Main peak | LMW | Antibody | HMW | Main peak | LMW |
| H-2B2-IgG1-ADC | 1.71 | 98.29 | / | H-2B2-IgG1 | 2.17 | 97.83 | / |
| H-3C8-IgG1-ADC | / | 100.0 | / | H-3C8-IgG1 | / | 100.0 | / |
| H-1H2-IgG1-ADC | / | 100.0 | / | H-1H2-IgG1 | / | 100.0 | / |
| T-6F7-IgG1-SI-ADC | 2.33 | 94.53 | 3.13 | T-6F7-IgG1-SI | 0.81 | 98.15 | 1.04 |
| H-2B2-CD28-ADC | 3.41 | 69.93 | 26.66 | H-2B2-CD28 | 3.87 | 77.39 | 18.74 |
| H-3C8-CD28-ADC | 1.80 | 74.61 | 23.59 | H-3C8-CD28 | 2.71 | 79.96 | 17.33 |
| CD28-T-6F7-ADC | 1.97 | 71.04 | 26.99 | CD28-T-6F7 | 5.41 | 81.87 | 12.72 |
| CD28-H-1H2-ADC | 1.18 | 94.41 | 4.41 | CD28-H-1H2 | 2.74 | 94.73 | 2.53 |
| H-1H2-T-6F7-ADC | 1.55 | 88.25 | 10.20 | H-1H2-T-6F7 | 2.38 | 93.40 | 4.22 |
| H-3C8-T-6F7-ADC | 2.28 | 90.16 | 7.56 | H-3C8-T-6F7 | 2.67 | 95.30 | 2.03 |
| H-2B2-T-6F7-ADC | 1.31 | 90.82 | 7.87 | H-2B2-T-6F7 | 1.93 | 92.55 | 5.52 |
| T-6F7-H-1H2-ADC | 0.70 | 97.77 | 1.53 | T-6F7-H-1H2 | 1.18 | 98.09 | 0.73 |
| Sacituzumab-SI-ADC | 5.16 | 94.84 | / | Sacituzumab-SI | 1.26 | 98.74 | / |
| Trastuzumab-ADC | 1.43 | 98.57 | / | Trastuzumab | 6.32 | 93.68 | / |
| Isotype-Control-ADC | 2.22 | 97.78 | / | Isotype Control | 3.29 | 96.71 | / |

The HIC-HPLC detection results are shown in Table 14. The results show that the DAR of ADC is about 3. Wherein the average DAR is determined by multiplying PA % (PA % is the peak area percentage as measured by the area under the 280 nm peak) multiplied by the corresponding drug load of 0, 2, 4, 6, or 8 and divided by 100. For example, the average DAR of 10 H-2B2-IgG1-ADC=[(7.81×0)+ (36.65×2)+ (39.81×4)+ (12.01×6)+ (3.71×8)]/100=3.34.

TABLE 14
HIC-HPLC test results

| | PA % at each DAR Peak | | | | | Average |
|---|---|---|---|---|---|---|
| | D0 | D2 | D4 | D6 | D8 | DAR |
| H-2B2-IgG1-ADC | 7.81 | 36.65 | 39.81 | 12.01 | 3.71 | 3.34 |
| H-3C8-IgG1-ADC | 4.69 | 31.40 | 43.70 | 15.22 | 4.99 | 3.69 |
| H-1H2-IgG1-ADC | 8.19 | 38.70 | 41.12 | 9.82 | 2.16 | 3.18 |
| T-6F7-IgG1-SI-ADC | 9.85 | 38.72 | 33.14 | 9.79 | 8.51 | 3.37 |
| H-2B2-CD28-ADC | 13.04 | 37.50 | 26.82 | 15.46 | 7.18 | 3.32 |
| H-3C8-CD28-ADC | 9.23 | 34.88 | 32.82 | 15.31 | 7.76 | 3.55 |
| CD28-T-6F7-ADC | 9.37 | 31.49 | 25.66 | 23.94 | 9.55 | 3.86 |
| CD28-H-1H2-ADC | 8.05 | 37.07 | 36.09 | 14.42 | 4.36 | 3.40 |
| H-1H2-T-6F7-ADC | 6.63 | 33.01 | 35.83 | 17.71 | 6.82 | 3.70 |
| H-3C8-T-6F7-ADC | 10.74 | 35.32 | 32.55 | 15.84 | 5.55 | 3.40 |
| H-2B2-T-6F7-ADC | 12.20 | 40.16 | 32.26 | 12.07 | 3.32 | 3.08 |
| T-6F7-H-1H2-ADC | 8.49 | 37.06 | 37.83 | 13.13 | 3.49 | 3.32 |
| Sacituzumab-SI-ADC | 15.41 | 42.55 | 28.41 | 7.71 | 5.92 | 2.92 |
| Trastuzumab-ADC | 4.52 | 28.80 | 40.08 | 18.08 | 8.52 | 3.95 |
| Isotype-Control-ADC | 10.42 | 36.75 | 36.59 | 12.91 | 3.32 | 3.24 |

### In Vitro Killing Activity

Different concentrations of purified ADC (10000 ng/mL, 2000 ng/ml, 400 ng/ml, 80 ng/mL, 16 ng/ml, 3.2 ng/mL, 0.64 ng/mL, 0.13 ng/mL) were used to treat human gastric cancer cell line NCI-N87 or human lung cancer cell line NCI-H292 cultured in a cell culture plate, and the killing activity was detected after 72 h of incubation with IncuCyte (Sartorius AG, IncuCyte® S3). The results are shown in the table below.

TABLE 15
In vitro killing results

| | IC50(ng/mL) | |
|---|---|---|
| ADC | NCI-N87 | NCI-H292 |
| H-3C8-IgG1-ADC | 2.56 | 65581 |
| H-2B2-IgG1-ADC | 2.24 | 2743 |
| H-1H2-IgG1-ADC | 3.96 | 20813 |
| T-6F7-IgG1-SI-ADC | 11.2 | 126 |
| H-2B2-CD28-ADC | 265 | 2853 |
| H-3C8-CD28-ADC | 82.7 | 2945 |
| CD28-T-6F7-ADC | 1500 | 1505 |
| CD28-H-1H2-ADC | 128 | 3260 |
| H-1H2-T-6F7-ADC | 21.8 | 929 |
| H-2B2-T-6F7-ADC | 22.7 | 167 |
| H-6F7-T-1H2-ADC | 19.8 | 675.5 |
| H-3C8-T-6F7-ADC | 17.6 | 208.5 |
| Trastuzumab-ADC | 14.8 | 289288 |
| Isotype Control | 2180 | 36207 |

In the NCI-N87 cell line, the HER2/TROP2 bispecific antibody ADC showed stronger killing ability, which is stronger than the control bispecific antibody ADC (IC50>10 times). In the NCI-H292 cell line, the HER2/TROP2 bispecific antibody ADC Killing is lower than TROP2 monoclonal antibody ADC, but stronger than HER2 monoclonal antibody ADC and control bispecific antibody ADC. The data showed that the HER2/TROP2 bispecific antibody ADC selectively killed HER2/TROP2 dual-target-expressing cell lines. In addition, the in vitro cell killing ability of H-2B2-T-6F7-ADC was similar to that of the parental T-6F7-IgG1-SI-ADC, suggesting that H-2B2-T-6F7-ADC has potential efficacy against TROP2-high/HER2-low tumor.

### Example 6. Anti-Tumor Activity in NCI-H1975 Xenograft Model

The ADC were tested for their effect on tumor growth in vivo in a model of lung adenocarcinoma. About 5×10⁶ NCI-H1975 (lung adenocarcinoma cells) were injected subcutaneously in B-NDG mice (Biocytogen Pharmaceuticals (Beijing) Co., Ltd., Beijing, China; Cat #B-CM-002). When the tumors in the mice reached a volume of about 100 mm³, the mice were randomly placed into different groups based on the volume of the tumor. The mice were then injected with phosphate buffer saline (PBS) or ADC by intravenous (i.v.) administration.

The injected volume was calculated based on the weight of the mouse and desired dosage of 3 mg/kg. The length of the long axis and the short axis of the tumor were measured and the volume of the tumor was calculated as 0.5× (long axis)×(short axis) 2. The weight of the mice was also measured twice a week.

The tumor growth inhibition percentage (TGI %) was calculated using the following formula: TGI (%)=[1−(Ti−T0)/(Vi−V0)]×100. Ti is the average tumor volume in the treatment group on day i. T0 is the average tumor volume in the treatment group on day zero. Vi is the average tumor volume in the control group on day i. V0 is the average tumor volume in the control group on day zero.

T-test was performed for statistical analysis. A TGI % higher than 60% indicates clear suppression of tumor growth. P<0.05 is a threshold to indicate significant difference.

TABLE 16
Group assignment

| | No. | | | | | Total No. |
|---|---|---|---|---|---|---|
| Group | of mice | ADC | Dosage | Route | Frequency | of administration |
| G1 | 5 | PBS | — | i.v. | QW | 2 |
| G2 | 5 | Isotype-Control-ADC | 3 mg/kg | i.v. | QW | 2 |
| G3 | 5 | Trastuzumab-ADC | 3 mg/kg | i.v. | QW | 2 |
| G4 | 5 | Sacituzumab-SI-ADC | 3 mg/kg | i.v. | QW | 2 |
| G5 | 5 | T-6F7-H-1H2-ADC | 3 mg/kg | i.v. | QW | 2 |
| G6 | 5 | H-3C8-T-6F7-ADC | 3 mg/kg | i.v. | QW | 2 |
| G7 | 5 | H-2B2-T-6F7-ADC | 3 mg/kg | i.v. | QW | 2 |
| G8 | 5 | H-1H2-IgG1-ADC | 3 mg/kg | i.v. | QW | 2 |
| G9 | 5 | H-2B2-IgG1-ADC | 3 mg/kg | i.v. | QW | 2 |
| G10 | 5 | H-3C8-IgG1-ADC | 3 mg/kg | i.v. | QW | 2 |
| G11 | 5 | T-6F7-IgG1-ADC | 3 mg/kg | i.v. | QW | 2 |
| G12 | 5 | CD28-H-1H2-ADC | 3 mg/kg | i.v. | QW | 2 |
| G13 | 5 | H-3C8-CD28-ADC | 3 mg/kg | i.v. | QW | 2 |
| G14 | 5 | H-2B2-CD28-ADC | 3 mg/kg | i.v. | QW | 2 |
| G15 | 5 | CD28-T-6F7-ADC | 3 mg/kg | i.v. | QW | 2 |

The weight of mice in different groups all increased. On the day of group assignment (Day 0), the average weight of each group was in the range of 22.0 g-22.6 g; At the end of the experiment (Day 28), the average weight of each group was in the range of 21.3 g-24.1 g, the average weight of each group was in the range of 94.1%-112.2%. The results showed that the tested ADC were well tolerated and were not obviously toxic to the mice.

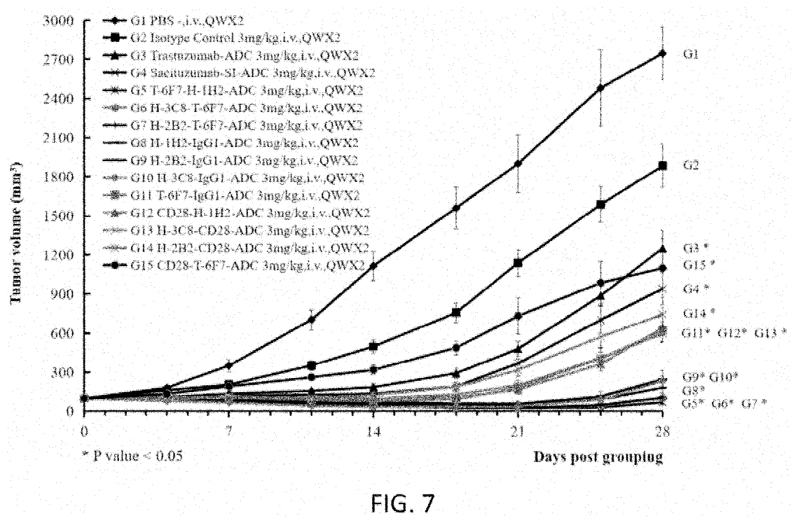

The tumor size in groups treated with the ADC are shown in FIG. 7. The table below summarizes the results for this experiment, including the tumor volumes on the day of grouping (day 0), 11 days after the grouping (day 11), 21 days after the grouping (day 21), and at the end of the experiment (day 28); the survival rate of the mice; Tumor Growth Inhibition value (TGI); and the statistical differences (P value) of tumor volume and body weight between the treatment and control groups.

TABLE 17
Tumor size changes

| | | | | | | | | P value | |
|---|---|---|---|---|---|---|---|---|---|
| | | Tumor volume (mm³) | | | | | | Body | Tumor |
| | | Day 0 | Day 11 | Day 21 | Day28 | Survival | % | weight | Volume |
| Control | G1 | 99 ± 3 | 702 ± 77 | 1898 ± 221 | 2745 ± 204 | 4/5 | NA | NA | NA |
| | G2 | 99 ± 3 | 352 ± 35 | 1138 ± 101 | 1882 ± 167 | 5/5 | 32.6 | 0.175 | 0.013 |
| Treat | G3 | 99 ± 3 | 158 ± 20 | 478 ± 57 | 1250 ± 135 | 5/5 | 56.5 | 0.088 | 3.86E−04 |
| | G4 | 99 ± 3 | 128 ± 24 | 367 ± 36 | 940 ± 121 | 5/5 | 68.2 | 0.021 | 9.16E−05 |
| | G5 | 99 ± 3 | 70 ± 9 | 23 ± 3 | 68 ± 17 | 5/5 | 101.2 | 0.376 | 1.50E−06 |
| | G6 | 99 ± 4 | 51 ± 5 | 25 ± 2 | 101 ± 15 | 5/5 | 99.9 | 0.287 | 1.62E−06 |
| | G7 | 99 ± 4 | 56 ± 9 | 24 ± 3 | 106 ± 10 | 5/5 | 99.7 | 0.478 | 1.61E−06 |
| | G8 | 99 ± 4 | 80 ± 17 | 54 ± 24 | 181 ± 36 | 5/5 | 96.9 | 0.965 | 2.29E−06 |
| | G9 | 99 ± 4 | 67 ± 20 | 54 ± 14 | 245 ± 67 | 5/5 | 94.5 | 0.986 | 4.02E−06 |
| | G10 | 99 ± 4 | 41 ± 3 | 38 ± 6 | 234 ± 35 | 5/5 | 94.9 | 0.735 | 2.62E−06 |
| | G11 | 99 ± 4 | 87 ± 7 | 181 ± 35 | 619 ± 40 | 5/5 | 80.4 | 0.040 | 8.45E−06 |
| | G12 | 99 ± 3 | 101 ± 29 | 166 ± 31 | 640 ± 110 | 5/5 | 79.6 | 0.418 | 2.74E−05 |
| | G13 | 99 ± 3 | 90 ± 29 | 198 ± 38 | 595 ± 60 | 5/5 | 81.3 | 0.126 | 9.94E−06 |
| | G14 | 99 ± 4 | 104 ± 13 | 316 ± 49 | 743 ± 56 | 5/5 | 75.7 | 0.014 | 1.51E−05 |
| | G15 | 99 ± 3 | 261 ± 24 | 731 ± 137 | 1097 ± 226 | 5/5 | 62.3 | 0.109 | 1.16E−03 |

The tumor volumes in all treatment groups (G3-G15) were smaller than those in the control group (G1 and G2). The treatment groups had different tumor inhibitory effects. Three bispecific antibody ADC, including T-6F7-H-1H2-ADC (G5), H-3C8-T-6F7-ADC (G6) and H-2B2-T-6F7-ADC (G7), showed sustained and potent tumor suppression effects.

Corresponding monoclonal antibody ADC (G8-G11) and control ADC (G12-G14) except G15 also had a strong tumor suppressor effect. Isotype Control (G2) also showed a strong tumor-inhibiting effect, which may be due to unstable linker or non-specific binding of Isotype Control, which resulted in the release of vc-MMAE in mice, resulting in non-specific killing.

In another experiment, about 5×10⁶ NCI-H1975 cells were injected subcutaneously in B-NDG mice, and when the tumor volume grew to about 300 mm³, the mice were divided to a control group and different treatment groups based on tumor size (5 mice per group). The treatment groups were randomly selected for H-2B2-T-6F7 treatment (G2 (3 mg/kg) and G3 (10 mg/kg)), H-2B2-T-6F7-ADC treatment (G4, 3 mg/kg) or MMAE treatment (G5, 0.06 mg/kg, equimolar dosage to G4). The control group mice were injected with PBS (G1). The frequency of administration was once a week (2 times of administrations in total). The tumor volume was measured twice a week and the body weight of the mice was weighed as well.

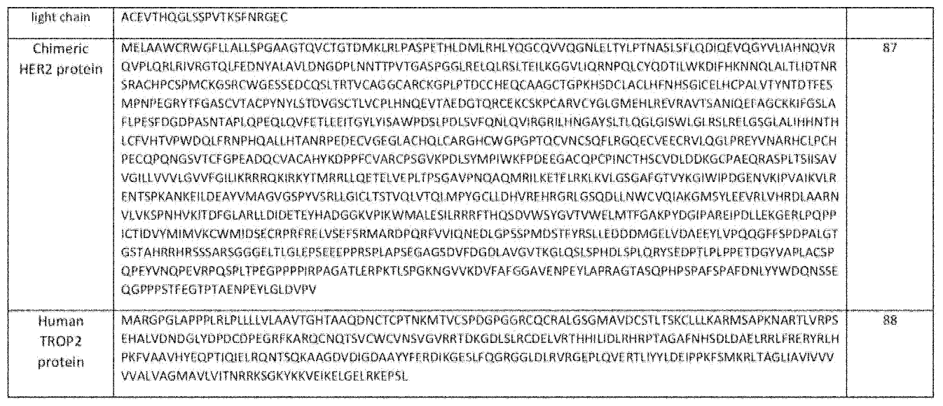

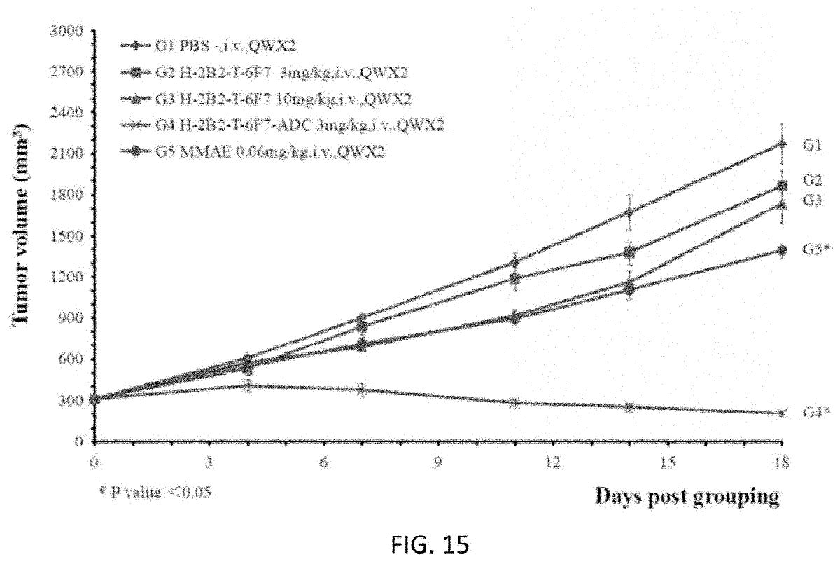

The tumor sizes in groups treated with the antibodies are shown in FIG. 15 and Table 18, which show that compared with the control group, the tumor growth in the treatment groups were inhibited to different extents, and the anti-HER2/TROP2 bispecific antibody ADC H-2B2-T-6F7-ADC at a dose of 3 mg/kg obtained better tumor inhibitory effect as compared to anti-HER2/TROP2 bispecific antibody H-2B2-T-6F7 at a dose of 10 mg/kg. MMAE treatment group obtained limited inhibition of tumor growth, suggesting that the in vivo efficacy of H-2B2-T-6F7-ADC depended on the binding of HER2 and TROP2 antigen.

TABLE 18
Tumor size changes

| | Tumor volume (mm³) | | | | TGI | P value of |
|---|---|---|---|---|---|---|
| Group | Day 0 | Day 11 | Day 18 | Survival | % | Tumor Volume |
| G1 | 307 ± 5 | 899 ± 29 | 2169 ± 145 | 5/5 | NA | NA |
| G2 | 308 ± 11 | 833 ± 57 | 1862 ± 119 | 5/5 | 16.5 | 0.142 |
| G3 | 307 ± 9 | 685 ± 18 | 1735 ± 141 | 5/5 | 23.3 | 0.065 |
| G4 | 308 ± 10 | 373 ± 47 | 201 ± 20 | 5/5 | 105.7 | 9.18E−07 |
| G5 | 307 ± 6 | 710 ± 43 | 1391 ± 49 | 5/5 | 41.8 | 0.001 |

In another experiment, about 1×10⁶ NCI-H1975 cells were injected subcutaneously in B-NDG mice, and when the tumor volume grew to about 300 mm³, the mice were divided to different treatment groups based on tumor size. Details of the administration scheme are shown in the table below.

TABLE 19
Group assignment

| | No. of | | Dosage | | | Total No. of |
|---|---|---|---|---|---|---|
| Group | mice | Antibodies | (mg/kg) | Route | Frequency | administration |
| G1 | 5 | PBS | — | i.v. | QW | 2 |
| G2 | 5 | H-2B2-T-6F7-ADC | 0.3 mg/kg | i.v. | QW | 2 |
| G3 | 5 | H-2B2-T-6F7-ADC | 1 mg/kg | i.v. | QW | 2 |
| G4 | 5 | H-2B2-T-6F7-ADC | 3 mg/kg | i.v. | QW | 2 |
| G5 | 5 | Sacituzumab govitecan analog | 1 mg/kg | i.v. | QW | 2 |
| G6 | 5 | Sacituzumab govitecan analog | 3 mg/kg | i.v. | QW | 2 |
| G7 | 5 | Disitamab vedotin | 1 mg/kg | i.v. | QW | 2 |
| G8 | 5 | Disitamab vedotin | 3 mg/kg | i.v. | QW | 2 |
| G9 | 5 | Trastuzumab deruxtecan | 1 mg/kg | i.v. | QW | 2 |
| G10 | 5 | Trastuzumab deruxtecan | 3 mg/kg | i.v. | QW | 2 |

Disitamab vedotin (RC48, RemeGen Co., Ltd., VH SEQ ID NO: 83; VL SEQ ID NO: 84) is an ADC comprising a recombinant humanized monoclonal antibody against HER2 and a MMAE payload developed by RemeGen. In 2021, the product was conditionally approved in China for the treatment of HER2-expressing locally advanced/metastatic gastric cancer (gastroesophageal junction carcinoma).

Trastuzumab deruxtecan (Enhertu®, Daiichi Sankyo Company, Limited., VH SEQ ID NO: 85; VL SEQ ID NO: 86) is an ADC comprised of a humanized anti-HER2 antibody attached to a topoisomerase I inhibitor payload via a tetrapeptide linker. The product was first launched in 2020 in the U.S. for the treatment of adult patients with unresectable or metastatic HER2-positive breast cancer who have received two or more prior anti-HER2-based regimens in the metastatic setting.

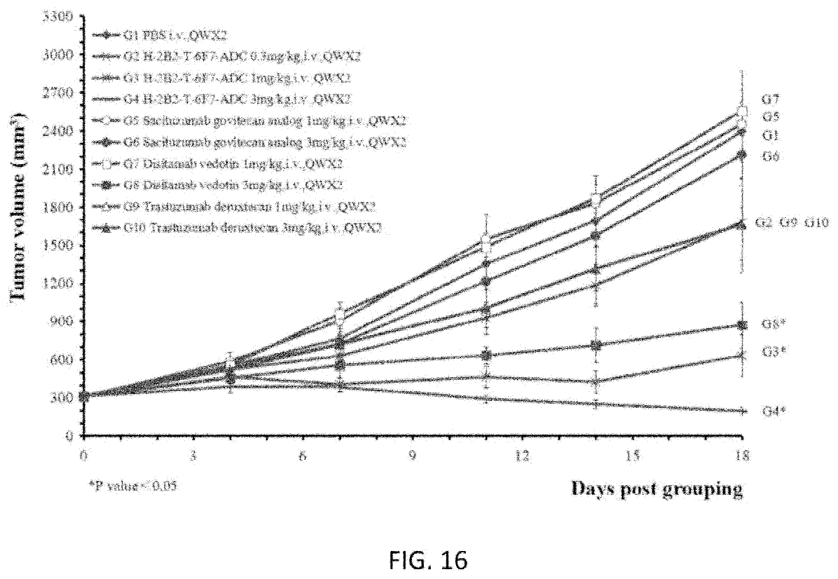

The tumor volume was measured twice a week and the results are shown in FIG. 16 and Table 20, which show that at the same dosage and frequency of administration, anti-HER2/TROP2 bispecific antibody ADC H-2B2-T-6F7-ADC inhibited tumor growth with a higher TGI % than that of Sacituzumab govitecan analog, Disitamab vedotin or Trastuzumab deruxtecan. In addition, the tumor inhibition of H-2B2-T-6F7-ADC showed a correlation trend with an increasing dose.

TABLE 20
Tumor size changes

| | | | | | | P value of |
|---|---|---|---|---|---|---|
| | Tumor volume (mm³) | | | | | Tumor |
| Group | Day 0 | Day 11 | Day 18 | Survival | TGI % | Volume |
| G1 | 308 ± 12 | 1351 ± 108 | 2391 ± 225 | 5/5 | NA | NA |
| G2 | 308 ± 19 | 925 ± 124 | 1678 ± 289 | 5/5 | 34.2 | 0.087 |
| G3 | 309 ± 17 | 464 ± 81 | 632 ± 167 | 5/5 | 84.5 | 2.35E−04 |
| G4 | 308 ± 22 | 289 ± 30 | 194 ± 26 | 5/5 | 105.5 | 1.05E−05 |
| G5 | 308 ± 14 | 1546 ± 196 | 2448 ± 424 | 5/5 | −2.7 | 0.909 |
| G6 | 309 ± 15 | 1216 ± 190 | 2210 ± 420 | 5/5 | 8.7 | 0.713 |
| G7 | 309 ± 16 | 1486 ± 111 | 2553 ± 167 | 5/5 | −7.7 | 0.580 |
| G8 | 309 ± 16 | 633 ± 65 | 869 ± 183 | 5/5 | 73.1 | 0.001 |
| G9 | 309 ± 16 | 1214 ± 206 | 2004 ± 369 | 5/5 | 18.6 | 0.396 |
| G10 | 309 ± 20 | 1004 ± 149 | 1660 ± 313 | 5/5 | 35.1 | 0.094 |

### Example 7. Anti-Tumor Activity in SK-OV-3 Xenograft Model

The ADCs were tested for their effect on tumor growth in vivo in a model of ovarian cancer. About 5×10⁶ SK-OV-3 cells were injected subcutaneously in B-NDG mice. When the tumors in the mice reached a volume of about 200-250 mm³, the mice were randomly placed into different groups based on the volume of the tumor. The mice were then injected with PBS or ADC by intravenous (i.v.) administration. The frequency of administration was once a week (3 administrations in total). The dosage of the first and second administration was both 3 mg/kg, and the last one was 5 mg/kg. Details of the administration scheme are shown in the table below.

TABLE 21
Group assignment

| | No. of | | | | Total No. of |
|---|---|---|---|---|---|
| Group | mice | Antibodies | Route | Frequency | administration |
| G1 | 5 | PBS | i.v. | QW | 3 |
| G2 | 5 | H-2B2-T-6F7-ADC | i.v. | QW | 3 |
| G3 | 5 | H-2B2-IgG1-ADC | i.v. | QW | 3 |
| G4 | 5 | T-6F7-IgG1-SI-ADC | i.v. | QW | 3 |
| G5 | 5 | Trastuzumab | i.v. | QW | 3 |
| | | deruxtecan analog | | | |
| G6 | 5 | DS-1062 analog | i.v. | QW | 3 |

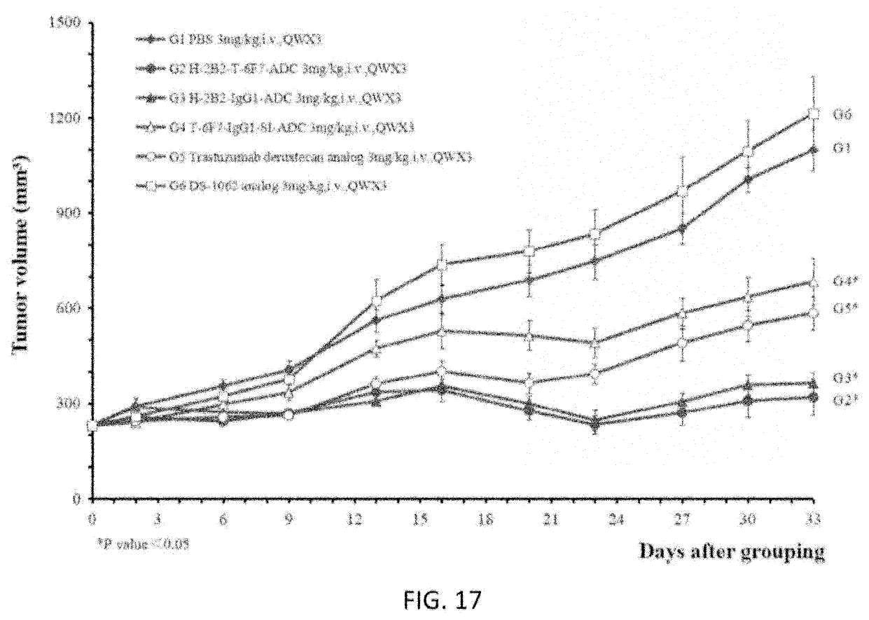

The tumor volume was measured twice a week and the results are shown in FIG. 17 and Table 22, which show that anti-HER2/TROP2 bispecific antibody ADC H-2B2-T-6F7-ADC had the highest tumor growth inhibition at 3 mg/kg in ovarian cancer model. In addition, anti-HER2 monoclonal antibody ADC H-2B2-IgG1-ADC obtained better efficacy than positive control Trastuzumab deruxtecan analog, and anti-TROP2 monoclonal antibody ADC H-2B2-T-6F7-ADC obtained better efficacy than positive control DS-1062 analog.

TABLE 22
Tumor size changes

| | Tumor volume (mm3) | | | | | P value of |
|---|---|---|---|---|---|---|
| Group | Day 0 | Day 13 | Day 23 | Day 33 | TGI % | Tumor Volume |
| G1 | 230 ± 12 | 560 ± 36 | 746 ± 54 | 1097 ± 66 | NA | NA |
| G2 | 230 ± 12 | 334 ± 21 | 232 ± 29 | 319 ± 56 | 89.7 | 8.09E−06 |
| G3 | 230 ± 15 | 306 ± 16 | 246 ± 33 | 362 ± 34 | 84.7 | 2.56E−06 |
| G4 | 230 ± 15 | 472 ± 26 | 490 ± 46 | 682 ± 73 | 47.8 | 0.003 |
| G5 | 229 ± 15 | 361 ± 24 | 392 ± 32 | 583 ± 51 | 59.2 | 1.51E−04 |
| G6 | 230 ± 15 | 623 ± 68 | 833 ± 78 | 1213 ± 115 | −13.4 | 0.429 |

In another experiment, about 5×10⁶ SK-OV-3 cells were injected subcutaneously in B-NDG mice, and when the tumor volume grew to about 200-250 mm³, the mice were divided to a control group and four treatment groups based on tumor size (5 mice per group). The treatment group mice were randomly selected for intravenous (i.v.) administration of H-2B2-T-6F7-ADC at 0.3 mg/kg (G2), 1 mg/kg (G3), 3 mg/kg (G4) or 10 mg/kg (G5). The control group mice were injected with an equal volume of PBS (G1). The frequency of administration was once a week (2 times of administrations in total). The tumor volume was measured twice a week and the results are shown in Table 23, which show that H-2B2-T-6F7-ADC exhibit sustained tumor inhibition activity in a dose-dependent manner.

TABLE 23
Tumor size changes

| | Tumor volume (mm³) | | | | | P value of |
|---|---|---|---|---|---|---|
| Group | Day 0 | Day 11 | Day 21 | Day 35 | TGI % | Tumor Volume |
| G1 | 208 ± 8 | 497 ± 32 | 937 ± 61 | 1291 ± 115 | NA | NA |
| G2 | 207 ± 10 | 482 ± 41 | 752 ± 51 | 1169 ± 69 | 11.2 | 0.392 |
| G3 | 207 ± 13 | 378 ± 18 | 604 ± 19 | 952 ± 77 | 31.3 | 0.055 |
| G4 | 207 ± 12 | 321 ± 32 | 433 ± 47 | 568 ± 70 | 66.7 | 0.001 |
| G5 | 207 ± 12 | 132 ± 11 | 38 ± 7 | 59 ± 24 | 113.6 | 6.06E−06 |

### Example 8. Anti-Tumor Activity in NCI-N87 Model

The ADC were tested for their effect on tumor growth in vivo in a model of stomach cancer. About 5×10⁶ NCI-N87 cells were injected subcutaneously in B-NDG mice. When the tumors in the mice reached a volume of about 150 mm³, the mice were randomly placed into different groups based on the volume of the tumor. The mice were then injected with PBS or ADC by intravenous (i.v.) administration. Details of the administration scheme are shown in the table below.

TABLE 24
Group assignment

| | No. of | | Dosage | | | Total No. of |
|---|---|---|---|---|---|---|
| Group | mice | Antibodies | (mg/kg) | Route | Frequency | administration |
| G1 | 5 | PBS | — | i.v. | QW | 1 |
| G2 | 5 | H-2B2-T-6F7-ADC | 1 mg/kg | i.v. | QW | 1 |
| G3 | 5 | H-2B2-T-6F7-ADC | 3 mg/kg | i.v. | QW | 1 |
| G4 | 5 | H-2B2-IgG1-ADC | 1 mg/kg | i.v. | QW | 1 |
| G5 | 5 | H-2B2-IgG1-ADC | 3 mg/kg | i.v. | QW | 1 |
| G6 | 5 | T-6F7-IgG1-SI-ADC | 1 mg/kg | i.v. | QW | 1 |
| G7 | 5 | T-6F7-IgG1-SI-ADC | 3 mg/kg | i.v. | QW | 1 |
| G8 | 5 | Sacituzumab govitecan analog | 1 mg/kg | i.v. | QW | 1 |
| G9 | 5 | Sacituzumab govitecan analog | 3 mg/kg | i.v. | QW | 1 |
| G10 | 5 | Disitamab vedotin | 1 mg/kg | i.v. | QW | 1 |
| G11 | 5 | Disitamab vedotin | 3 mg/kg | i.v. | QW | 1 |
| G12 | 5 | Trastuzumab deruxtecan | 1 mg/kg | i.v. | QW | 1 |
| G13 | 5 | Trastuzumab deruxtecan | 3 mg/kg | i.v. | QW | 1 |

The tumor volume was measured twice a week and the results are shown in Table 25, which show that anti-HER2/TROP2 bispecific antibody ADC H-2B2-T-6F7-ADC had the highest tumor growth inhibition at 3 mg/kg in stomach cancer NCI-N87 model. Among the anti-TROP2 monoclonal antibody ADC, T-6F7-IgG1-SI-ADC obtained better efficacy than positive control Sacituzumab govitecan analog both at a dose of 1 mg/kg and 3 mg/kg. The anti-HER2 monoclonal antibody ADC H-2B2-IgG1-ADC also obtained better efficacy than positive control Disitamab vedotin and Trastuzumab deruxtecan both at a dose of 1 mg/kg and 3 mg/kg.

TABLE 25
Tumor size changes

| | Tumor volume (mm³) | | | | | P value of |
|---|---|---|---|---|---|---|
| Group | Day 0 | Day 11 | Day 21 | Day 32 | TGI % | Tumor Volume |
| G1 | 142 ± 3 | 269 ± 18 | 297 ± 21 | 409 ± 40 | NA | NA |
| G2 | 143 ± 4 | 141 ± 6 | 103 ± 7 | 133 ± 14 | 103.7 | 1.87E−04 |
| G3 | 142 ± 3 | 92 ± 5 | 42 ± 6 | 48 ± 6 | 135.2 | 2.10E−05 |
| G4 | 142 ± 4 | 153 ± 6 | 118 ± 11 | 181 ± 20 | 85.6 | 0.001 |
| G5 | 142 ± 6 | 91 ± 14 | 50 ± 11 | 64 ± 18 | 129.6 | 4.99E−05 |
| G6 | 143 ± 5 | 206 ± 22 | 197 ± 22 | 269 ± 32 | 52.6 | 0.026 |
| G7 | 142 ± 4 | 120 ± 4 | 84 ± 5 | 124 ± 13 | 106.8 | 1.50E−04 |
| G8 | 143 ± 4 | 222 ± 14 | 237 ± 25 | 337 ± 31 | 27.1 | 0.196 |
| G9 | 143 ± 5 | 193 ± 11 | 232 ± 17 | 313 ± 20 | 36.3 | 0.063 |
| G10 | 142 ± 5 | 207 ± 19 | 210 ± 22 | 335 ± 55 | 27.7 | 0.310 |
| G11 | 143 ± 4 | 122 ± 11 | 98 ± 15 | 116 ± 11 | 110.1 | 1.10E−04 |
| G12 | 143 ± 6 | 162 ± 7 | 147 ± 7 | 250 ± 13 | 59.7 | 0.006 |
| G13 | 143 ± 6 | 105 ± 9 | 89 ± 11 | 116 ± 20 | 109.9 | 1.87E−04 |

### Example 9. Anti-Tumor Activity in BT474 Model

The ADC were tested for their effect on tumor growth in vivo in a model of breast cancer. About 1×10⁷ BT-474 cells were injected subcutaneously in B-NDG mice. When the tumors in the mice reached a volume of about 200 mm³, the mice were randomly placed into different groups based on the volume of the tumor. The mice were then injected with PBS or ADC by intravenous (i.v.) administration. Details of the administration scheme are shown in the table below.

TABLE 26
Group assignment

| | No. of | | Dosage | | | Total No. of |
|---|---|---|---|---|---|---|
| Group | mice | Antibodies | (mg/kg) | Route | Frequency | administration |
| G1 | 6 | PBS | — | i.v. | QW | 2 |
| G2 | 6 | Isotype-Control-ADC | 3 mg/kg | i.v. | QW | 2 |
| G3 | 6 | H-2B2-T-6F7-ADC | 3 mg/kg | i.v. | QW | 2 |
| G4 | 6 | H-2B2-IgG1-ADC | 3 mg/kg | i.v. | QW | 2 |
| G5 | 6 | T-6F7-IgG1-SI-ADC | 3 mg/kg | i.v. | QW | 2 |
| G6 | 6 | Sacituzumab govitecan analog | 3 mg/kg | i.v. | QW | 2 |
| G7 | 6 | Disitamab vedotin | 3 mg/kg | i.v. | QW | 2 |
| G8 | 6 | Trastuzumab deruxtecan | 3 mg/kg | i.v. | QW | 2 |

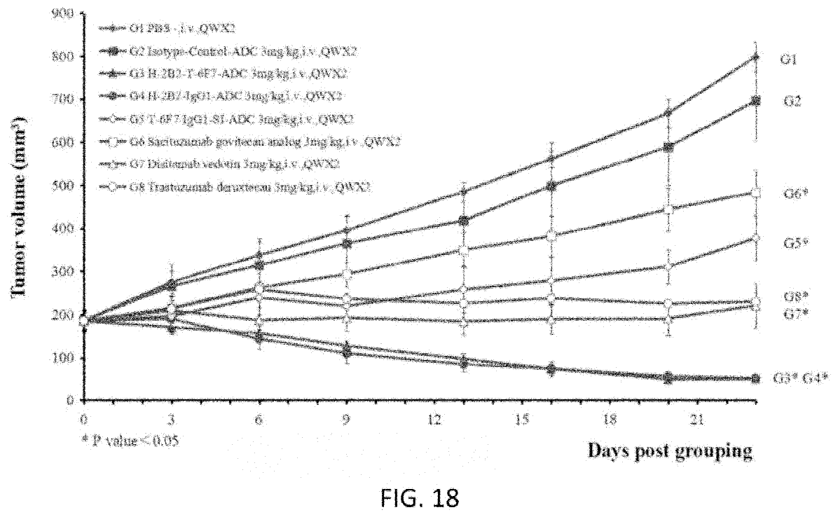

The tumor volume was measured twice a week and the results are shown in FIG. 18, which show that anti-HER2/TROP2 bispecific antibody ADC H-2B2-T-6F7-ADC had the highest tumor growth inhibition at 3 mg/kg in HER2-high expressing breast cancer BT-474 model (TGI %=122.0% on day 23). Among the anti-TROP2 monoclonal antibody ADC, T-6F7-IgG1-SI-ADC obtained better efficacy than positive control Sacituzumab govitecan analog (TGI %=68.7% and 51.4% respectively). The anti-HER2 monoclonal antibody ADC H-2B2-IgG1-ADC also obtained better efficacy than positive control Disitamab vedotin abd Trastuzumab deruxtecan (TGI %=121.9%, 94.4% and 92.9% respectively).

### Example 10. Anti-Tumor Activity in NCI-H292 Model

The ADC were tested for their effect on tumor growth in NCI-H292 lung cancer model. About 5×10⁶ NCI-H292 cells were injected subcutaneously in B-NDG mice. When the tumors in the mice reached a volume of about 300 mm³, the mice were randomly placed into different groups based on the volume of the tumor. The mice were then injected with PBS or ADC by intravenous (i.v.) administration. Details of the administration scheme are shown in the table below.

TABLE 27
Group assignment

| | No. of | | Dosage | | | Total No. of |
|---|---|---|---|---|---|---|
| Group | mice | Antibodies | (mg/kg) | Route | Frequency | administration |
| G1 | 5 | PBS | — | i.v. | QW | 1 |
| G2 | 5 | H-2B2-T-6F7-ADC | 1 mg/kg | i.v. | QW | 1 |
| G3 | 5 | H-2B2-T-6F7-ADC | 3 mg/kg | i.v. | QW | 2 |
| G4 | 5 | H-2B2-T-6F7-ADC | 10 mg/kg | i.v. | QW | 1 |
| G5 | 5 | Sacituzumab govitecan analog | 1 mg/kg | i.v. | QW | 1 |
| G6 | 5 | Sacituzumab govitecan analog | 3 mg/kg | i.v. | QW | 2 |
| G7 | 5 | Sacituzumab govitecan analog | 10 mg/kg | i.v. | QW | 1 |
| G8 | 5 | Trastuzumab deruxtecan | 1 mg/kg | i.v. | QW | 1 |
| G9 | 5 | Trastuzumab deruxtecan | 3 mg/kg | i.v. | QW | 2 |
| G10 | 5 | Trastuzumab deruxtecan | 10 mg/kg | i.v. | QW | 1 |

The tumor volume was measured twice a week and the results are shown in Table 28, which show that anti-HER2/TROP2 bispecific antibody ADC H-2B2-T-6F7-ADC inhibited tumor growth in a dose-dependent manner, and obtained the highest TGI % in treatment groups at 1 mg/kg, 3 mg/kg and 10 mg/kg respectively. The results indicates that anti-HER2/TROP2 bispecific antibody ADC H-2B2-T-6F7-ADC has therapeutic potential for the treatment of HER2-low expressing tumor.

TABLE 28
Tumor size changes

| | | | | | | P value of |
|---|---|---|---|---|---|---|
| | Tumor volume (mm³) | | | | | Tumor |
| Group | Day 0 | Day 11 | Day 21 | Day 32 | TGI % | Volume |
| G1 | 311 ± 14 | 918 ± 88 | 1492 ± 144 | 1914 ± 205 | NA | NA |
| G2 | 311 ± 20 | 656 ± 67 | 1097 ± 157 | 1484 ± 104 | 26.9 | 0.098 |
| G3 | 311 ± 25 | 452 ± 48 | 546 ± 81 | 925 ± 76 | 61.7 | 0.005 |
| G4 | 311 ± 19 | 140 ± 21 | 323 ± 38 | 605 ± 17 | 81.6 | 2.20E−04 |
| G5 | 311 ± 19 | 710 ± 51 | 1291 ± 149 | 1629 ± 161 | 17.8 | 0.305 |
| G6 | 311 ± 17 | 574 ± 46 | 801 ± 14 | 1132 ± 43 | 48.8 | 0.006 |
| G7 | 311 ± 20 | 487 ± 51 | 833 ± 39 | 1118 ± 37 | 49.7 | 0.005 |
| G8 | 311 ± 14 | 743 ± 68 | 1286 ± 71 | 1927 ± 156 | −0.7 | 0.963 |
| G9 | 311 ± 18 | 529 ± 45 | 714 ± 11 | 1141 ± 84 | 48.2 | 0.016 |
| G10 | 310 ± 18 | 252 ± 31 | 549 ± 31 | 899 ± 42 | 63.3 | 0.001 |

### Example 11. In Vivo Efficacy in Human Pancreatic Patient-Derived Xenograft (PDX) Model

The ADC were tested for their effect in two human pancreatic PDX (PDX001 and PDX002) models. Immunofluorescence staining of patient-derived pancreatic tumor fragments was performed and the images were analyzed via HALO 3.2 version. The results showed that HER2 positive cell and TROP2 positive cell in PDX001 were 84.72% and 89.09% respectively. In PDX002, HER2 positive cell and TROP2 positive cell were 86.34% and 89.17% respectively.

In PDX001 model, B-NDG mice were engrafted in the right flank with patient-derived pancreatic tumor fragments (2 mm×2 mm×2 mm). When the tumors in the mice reached a volume of about 300-400 mm³, the mice were randomly placed into different groups based on the volume of the tumor. The mice were then injected with PBS or ADC by intravenous (i.v.) administration. Details of the administration scheme are shown in the table below.

TABLE 29
Group assignment

| | No. of | | Dosage | | | Total No. of |
|---|---|---|---|---|---|---|
| Group | mice | Antibodies | (mg/kg) | Route | Frequency | administration |
| G1 | 6 | PBS | — | i.v. | QW | 1 |
| G2 | 6 | H-2B2-T-6F7-ADC | 5 mg/kg | i.v. | QW | 1 |
| G3 | 6 | H-2B2-IgG1-ADC | 5 mg/kg | i.v. | QW | 1 |
| G4 | 6 | T-6F7-IgG1-SI-ADC | 5 mg/kg | i.v. | QW | 1 |
| G5 | 6 | Sacituzumab govitecan analog | 5 mg/kg | i.v. | QW | 1 |
| G6 | 6 | Disitamab vedotin | 5 mg/kg | i.v. | QW | 1 |
| G7 | 6 | Trastuzumab deruxtecan | 5 mg/kg | i.v. | QW | 1 |

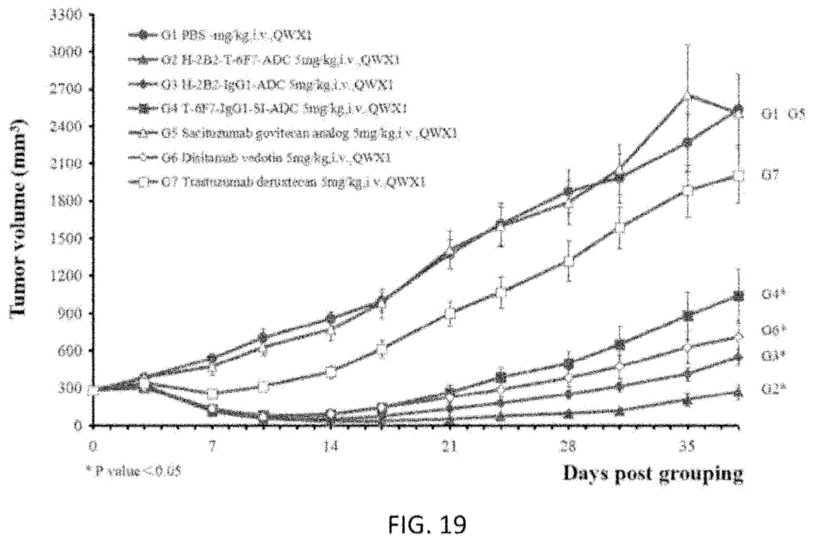

The tumor volume was measured twice a week and the results are shown in FIG. 19, which show that treatment with H-2B2-T-6F7-ADC (G2), H-2B2-IgG1-ADC (G3) and T-6F7-IgG1-SI-ADC (G4) resulted in significant tumor growth inhibition in HER2/TROP2 co-expressing human pancreatic PDX model, with TGI % of 100.5%, 88.2% and 66.4% respectively on Day 38 (38 days after the grouping).

In PDX002 model, when the tumors in the mice reached a volume of about 250-300 mm³, the mice were divided to a control group and different treatment groups based on tumor size (5 mice per group). The treatment groups were randomly selected for 3 mg/kg H-2B2-T-6F7-ADC treatment (G2), H-2B2-IgG1-ADC treatment (G3), T-6F7-IgG1-SI-ADC treatment (G4), Sacituzumab govitecan analog treatment (G5), Disitamab vedotin treatment (G6) or Trastuzumab deruxtecan treatment (G7). The control group mice were injected with PBS (G1). The frequency of administration was once a week (1 times of administrations in total).

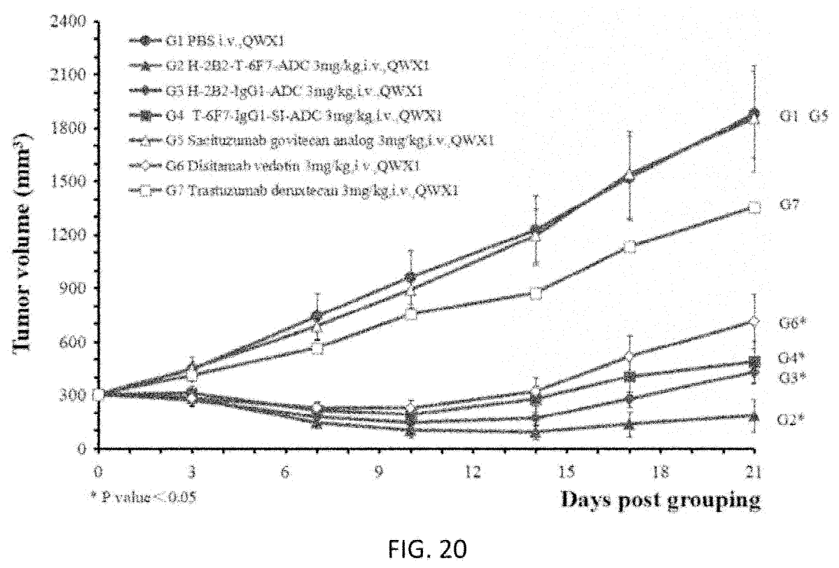

The tumor size in groups treated with the ADC are shown in FIG. 20. Consistent with the in vivo findings above, H-2B2-T-6F7-ADC (G2), H-2B2-IgG1-ADC (G3) and T-6F7-IgG1-SI-ADC (G4) resulted in a substantial antitumor activity, with TGI % of 107.5%, 91.9% and 88.2% respectively on Day 21 (21 days after the grouping).

### Example 12. Toxicological Studies

### Toxicological Study in B-NDG Mice

The toxicity of the anti-HER2/TROP2 bispecific antibody ADC were determined in B-NDG mice. Specifically, the mice were placed into six groups (12 mice per group), administered with physiological saline (G1) or H-2B2-T-6F7-ADC at 10 mg/kg (G2), 30 mg/kg (G3), 50 mg/kg (G4), 70 mg/kg (G5) or 100 mg/kg (G6) by intravenous injection. The frequency of administration was once a week (1 administrations in total). Details of the administration scheme are shown in the table below.

TABLE 30
Group assignment

| | | | | | | Total No. of |
|---|---|---|---|---|---|---|
| Group | No. of mice | Antibodies | Dosage (mg/kg) | Route | Frequency | administration |
| G1 | 12 | Saline | — | i.v. | Single dose | 1 |
| G2 | 12 | H-2B2-T-6F7-ADC | 10 mg/kg | i.v. | Single dose | 1 |
| G3 | 12 | H-2B2-T-6F7-ADC | 30 mg/kg | i.v. | Single dose | 1 |
| G4 | 12 | H-2B2-T-6F7-ADC | 50 mg/kg | i.v. | Single dose | 1 |
| G5 | 12 | H-2B2-T-6F7-ADC | 70 mg/kg | i.v. | Single dose | 1 |
| G6 | 12 | H-2B2-T-6F7-ADC | 100 mg/kg | i.v. | Single dose | 1 |

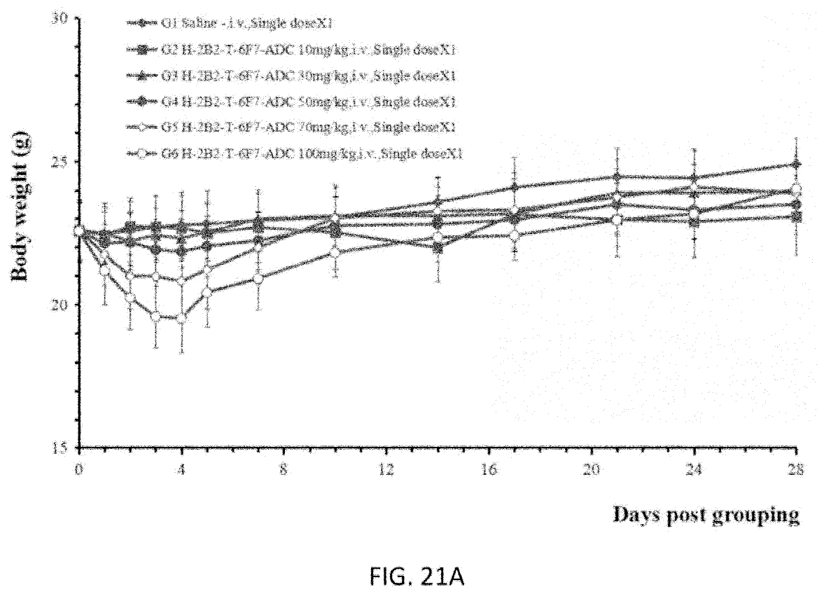

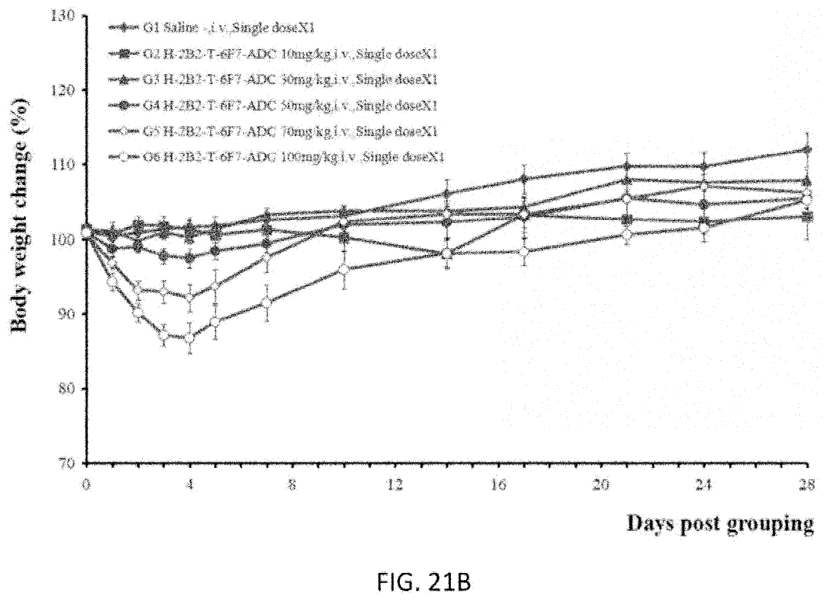

The body weight was measured every day in the first week, then twice a week until the end of the experiment after 4 weeks. As shown in FIGS. 21A-21B, the G5 group and G6 group mice showed significant weight loss, and the body weight of mice in other treatment groups showed no significant difference as compared with the control group mice. One mouse death occurred in the G6 group receiving 100 mg/kg of H-2B2-T-6F7-ADC.

Toxicological Studies in C57BL/6 Mice and hHER2/TROP2 Mice

Similar to the previous experiment, the toxicity of the anti-HER2/TROP2 bispecific antibody ADC in C57BL/6 mice and hHER2/TROP2 mice were determined.

hHER2/TROP2 mice was generated by crossing a HER2 humanized mice with a TROP2 humanized mice. The HER2 humanized mice was engineered to express a chimeric HER2 protein (SEQ ID NO: 87) wherein the extracellular and transmembrane region of the mouse HER2 protein was replaced with the corresponding human HER2 extracellular and transmembrane region. The TROP2 humanized mice was engineered to express human TROP2 protein (SEQ ID NO: 88) wherein the coding region of the mouse TROP2 gene was replaced with the corresponding human TROP2 coding region. A detailed description regarding the HER2 humanized mice and the TROP2 humanized mice can be found in CN202110959814.8 and CN202111119814.3; each of which is incorporated herein by reference in its entirety.

The hHER2/TROP2 mice and C57BL/6 mice were placed into different groups (6 mice per group) based on the body weight, administered with physiological saline, H-2B2-T-6F7-ADC (10 mg/kg, 30 mg/kg, 90 mg/kg) or MMAE (0.19 mg/kg, 0.57 mg/kg, 1.14 mg/kg and 1.71 mg/kg, equimolar amounts of H-2B2-T-6F7-ADC at 10 mg/kg, 30 mg/kg, 60 mg/kg and 90 mg/kg, respectively) by intravenous injection. The frequency of administration was once a week (1 administrations in total). Details of the administration scheme and survival on day 7 are shown in the table below.

TABLE 31
Group assignment

| | | | | Dosage | | |
|---|---|---|---|---|---|---|
| Group | No. of mice | Mice | Antibodies | (mg/kg) | Frequency | Survival |
| G1 | 6 | C57BL/6 mice | Saline | — | QW | 6/6 |
| G2 | 6 | C57BL/6 mice | H-2B2-T-6F7-ADC | 10 mg/kg | QW | 6/6 |
| G3 | 6 | C57BL/6 mice | H-2B2-T-6F7-ADC | 30 mg/kg | QW | 6/6 |
| G4 | 6 | C57BL/6 mice | H-2B2-T-6F7-ADC | 90 mg/kg | QW | 6/6 |
| G5 | 6 | C57BL/6 mice | MMAE | 0.19 mg/kg | QW | 6/6 |
| G6 | 6 | C57BL/6 mice | MMAE | 0.57 mg/kg | QW | 6/6 |
| G7 | 6 | C57BL/6 mice | MMAE | 1.14 mg/kg | QW | 5/6 |
| G8 | 6 | C57BL/6 mice | MMAE | 1.71 mg/kg | QW | 5/6 |
| G9 | 6 | hHER2/TROP2 mice | H-2B2-T-6F7-ADC | 10 mg/kg | QW | 6/6 |
| G10 | 6 | hHER2/TROP2 mice | H-2B2-T-6F7-ADC | 30 mg/kg | QW | 6/6 |
| G11 | 6 | hHER2/TROP2 mice | H-2B2-T-6F7-ADC | 90 mg/kg | QW | 5/6 |

The body weight was measured every day until the end of the experiment after 1 week. The results showed that all mice in the G1-G6 and G9-G10 groups survived. However, treatment groups G7, G8 and G11 showed some toxicity.

### Example 13. In Vivo Efficacy in Human Colorectal Cancer PDX Model

The ADCs were tested for the effect in human colorectal cancer patient-derived xenograft model. Immunofluorescence staining of patient-derived colorectal tumor fragments was performed and the images were analyzed via HALO 3.2 version. The results showed that the percentage of HER2 positive cells and TROP2 positive cells in human colorectal tumor tissue were 13.28% and 15.30% respectively. B-NDG mice were engrafted in the right flank with patient-derived colorectal tumor fragments (2 mm×2 mm×2 mm). When the tumors in the mice reached a volume of about 250-300 mm³, the mice were randomly placed into different groups based on the volume of the tumor. The mice were then injected with PBS or ADCs by intravenous (i.v.) administration. Details of the administration scheme are shown in the table below.

TABLE 32
Group assignment

| | No. of | | Dosage | | | Total No. of |
|---|---|---|---|---|---|---|
| Group | mice | Antibodies | (mg/kg) | Route | Frequency | administration |
| G1 | 5 | PBS | — | i.v. | QW | 2 |
| G2 | 5 | H-2B2-T-6F7-ADC | 6 mg/kg | i.v. | QW | 2 |
| G3 | 5 | Sacituzumab govitecan | 10 mg/kg | i.v. | BIW | 4 |
| G4 | 5 | Disitamab vedotin | 6 mg/kg | i.v. | QW | 2 |
| G5 | 5 | Trastuzumab deruxtecan | 6 mg/kg | i.v. | QW | 2 |

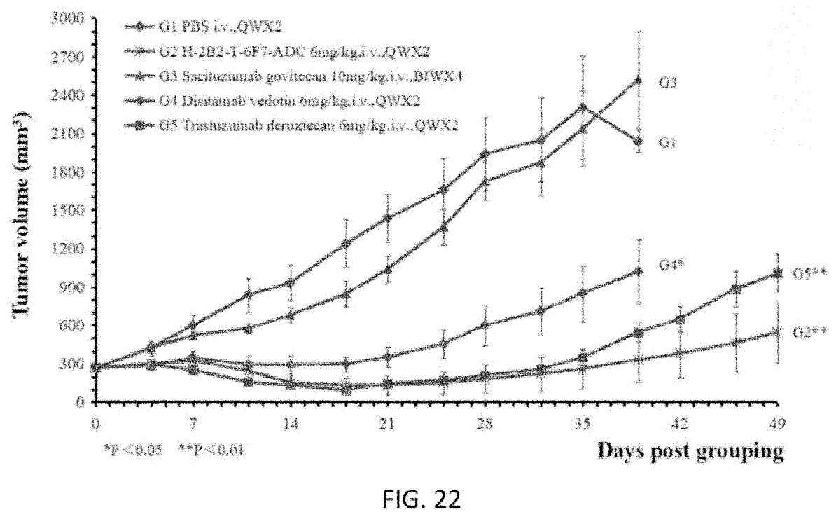

The tumor volume was measured twice a week and the results are shown in Table 33 and FIG. 22. Compared with PBS (G1) and Sacituzumab govitecan (G3), H-2B2-T-6F7-ADC (G2), Disitamab vedotin (G4) and Trastuzumab deruxtecan (G5) exhibited significant anti-tumor effects, and H-2B2-T-6F7-ADC inhibited tumor growth with a high TGI % of 96.4% on Day 39. In addition, H-2B2-T-6F7-ADC treatment group and Trastuzumab deruxtecan treatment group lasted for 49 days post grouping (Day 49), and H-2B2-T-6F7-ADC showed better tumor inhibition than Trastuzumab deruxtecan treatment group in human colorectal PDX model with HER2-low expression.

TABLE 33
Tumor size changes

| | | | | | | | P value |
|---|---|---|---|---|---|---|---|
| | Tumor volume (mm³) | | | | | TGI % | of Tumor |
| Group | Day 0 | Day 14 | Day 28 | Day 39 | Survival (Day 39) | (Day 39) | Volume |
| G1 | 270 ± 17 | 933 ± 141 | 1938 ± 287 | 2040 ± 88 | 3/5 | NA | NA |
| G2 | 270 ± 20 | 151 ± 54 | 181 ± 109 | 333 ± 172 | 5/5 | 96.4 | 3.71E−04 |
| G3 | 270 ± 18 | 682 ± 57 | 1726 ± 151 | 2520 ± 379 | 5/5 | −27.1 | 0.383 |
| G4 | 270 ± 19 | 291 ± 74 | 600 ± 154 | 1022 ± 249 | 5/5 | 57.5 | 0.024 |
| G5 | 270 ± 24 | 133 ± 10 | 212 ± 33 | 544 ± 77 | 5/5 | 84.5 | 1.67E−05 |

### Example 14. In Vivo Efficacy in Human Lung Cancer PDX Model

The ADCs were tested for the effect in human lung cancer patient-derived xenograft model. Immunofluorescence staining of patient-derived lung tumor fragments was performed and the images were analyzed via HALO 3.2 version. The results showed that the percentage of HER2 positive cells and TROP2 positive cells in human lung tumor tissue were 67.05% and 72.04% respectively. B-NDG mice were engrafted in the right flank with patient-derived lung tumor fragments (2 mm×2 mm×2 mm). When the tumors in the mice reached a volume of about 250-300 mm³, the mice were randomly placed into different groups based on the volume of the tumor. The mice were then injected with PBS or ADCs by intravenous (i.v.) administration. Details of the administration scheme are shown in the table below.

TABLE 34
Group assignment

| | No. of | | Dosage | | | Total No. of |
|---|---|---|---|---|---|---|
| Group | mice | Antibodies | (mg/kg) | Route | Frequency | administration |
| G1 | 5 | PBS | — | i.v. | QW | 2 |
| G2 | 5 | H-2B2-T-6F7-ADC | 3 mg/kg | i.v. | QW | 2 |
| G3 | 5 | H-2B2-T-6F7-ADC | 6 mg/kg | i.v. | QW | 2 |
| G4 | 5 | H-2B2-IgG1-ADC | 3 mg/kg | i.v. | QW | 2 |
| G5 | 5 | T-6F7-IgG1-SI-ADC | 3 mg/kg | i.v. | QW | 2 |
| G6 | 5 | Sacituzumab govitecan | 10 mg/kg | i.v. | BIW | 4 |
| G7 | 5 | Disitamab vedotin | 6 mg/kg | i.v. | QW | 2 |
| G8 | 5 | Trastuzumab deruxtecan | 6 mg/kg | i.v. | QW | 2 |

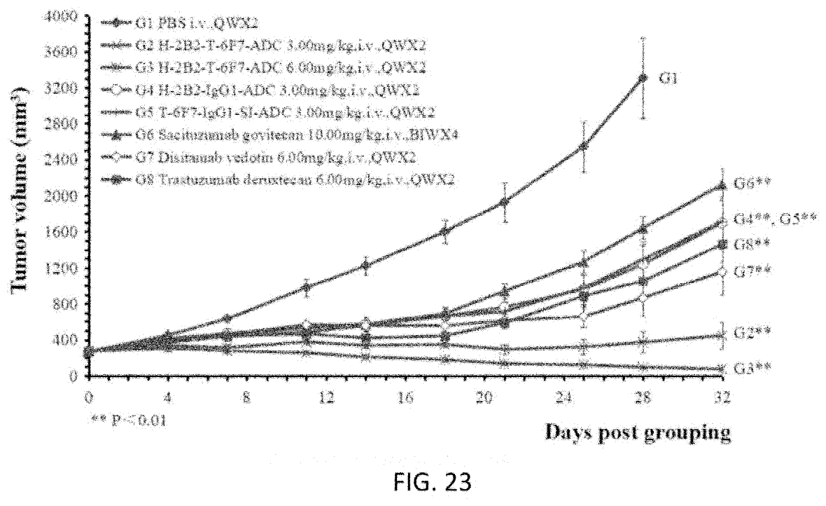

The tumor volume was measured twice a week and the results are shown in Table 35 and FIG. 23. H-2B2-T-6F7-ADC at 3 mg/kg (G2) obtained better anti-tumor activity than the parent monoclonal antibodies H-2B2-IgG1-ADC (G4) and T-6F7-IgG1-SI-ADC (G5). In addition, H-2B2-IgG1-ADC resulted in significant tumor growth inhibition even at a dosage of 3 mg/kg than the positive control Disitamab vedotin and Trastuzumab deruxtecan at a dosage of 6 mg/kg in HER2-low expressing human lung PDX model.

TABLE 35
Tumor size changes

| | Tumor volume (mm³) | | | TGI % | P value of |
|---|---|---|---|---|---|
| Group | Day 0 | Day 14 | Day 28 | (Day 28) | Tumor Volume |
| G1 | 270 ± 8 | 1221 ± 97 | 3309 ± 451 | NA | NA |
| G2 | 270 ± 15 | 337 ± 22 | 372 ± 116 | 96.7 | 2.06E−04 |
| G3 | 270 ± 15 | 208 ± 17 | 95 ± 11 | 105.8 | 8.40E−05 |
| G4 | 270 ± 11 | 572 ± 79 | 1238 ± 214 | 68.2 | 0.003 |
| G5 | 270 ± 10 | 558 ± 64 | 1291 ± 199 | 66.4 | 0.003 |
| G6 | 270 ± 14 | 568 ± 31 | 1639 ± 122 | 55.0 | 0.005 |
| G7 | 270 ± 13 | 555 ± 54 | 865 ± 207 | 80.4 | 0.001 |
| G8 | 270 ± 13 | 420 ± 11 | 1051 ± 167 | 74.3 | 0.001 |

The mouse tumor volume and survival were continued to be monitored after Day 28. At the end of the experiment on Day 63, all mice in groups G1-G8 (except G3) were euthanized due to excessive tumor volume, and all mice survived in group G3 with a mean tumor volume of 514±172 mm³, indicating that H-2B2-T-6F7-ADC has strong therapeutic potential.

### Example 15. In Vivo Efficacy in Human Gastric Cancer PDX Model

The ADCs were tested for the effect in human gastric cancer patient-derived xenograft model. Immunofluorescence staining of patient-derived gastric tumor fragments was performed and the results showed that the percentage of HER2 positive cells and TROP2 positive cells were 0.08% and 0.34% respectively. B-NDG mice were engrafted in the right flank with patient-derived gastric tumor fragments (2 mm×2 mm×2 mm). When the tumors in the mice reached a volume of about 150-200 mm³, the mice were randomly placed into different groups based on the volume of the tumor. The mice were then injected with PBS or ADCs by intravenous (i.v.) administration. Details of the administration scheme are shown in the table below.

TABLE 36
Group assignment

| | No. of | | Dosage | | | Total No. of |
|---|---|---|---|---|---|---|
| Group | mice | Antibodies | (mg/kg) | Route | Frequency | administration |
| G1 | 5 | PBS | — | i.v. | QW | 2 |
| G2 | 5 | H-2B2-T-6F7-ADC | 3 mg/kg | i.v. | QW | 2 |
| G3 | 5 | H-2B2-T-6F7-ADC | 6 mg/kg | i.v. | QW | 2 |
| G4 | 5 | Sacituzumab govitecan | 10 mg/kg | i.v. | BIW | 4 |
| G5 | 5 | Disitamab vedotin | 6 mg/kg | i.v. | QW | 2 |
| G6 | 5 | Trastuzumab deruxtecan | 6 mg/kg | i.v. | QW | 2 |

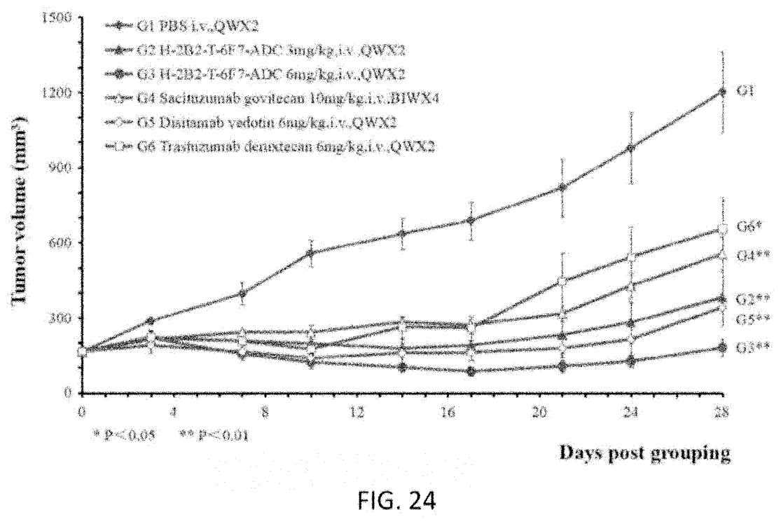

The tumor volume was measured twice a week and the results are shown in Table 37 and FIG. 24. The experimental results showed that H-2B2-T-6F7-ADC obtained better anti-tumor activity than that of the positive control Disitamab vedotin and Trastuzumab deruxtecan at a dosage of 6 mg/kg in HER2-low expressing human gastric PDX model.

TABLE 37
Tumor size changes

| | Tumor volume (mm³) | | | TGI % | P value of |
|---|---|---|---|---|---|
| Group | Day 0 | Day 14 | Day 28 | (Day 28) | Tumor Volume |
| G1 | 165 ± 13 | 635 ± 63 | 1202 ± 163 | NA | NA |
| G2 | 166 ± 13 | 177 ± 35 | 381 ± 116 | 79.3 | 0.004 |
| G3 | 165 ± 15 | 101 ± 20 | 180 ± 36 | 98.6 | 2.42E−04 |
| G4 | 166 ± 20 | 282 ± 20 | 554 ± 69 | 62.5 | 0.005 |
| G5 | 166 ± 18 | 160 ± 39 | 341 ± 56 | 83.2 | 0.001 |
| G6 | 166 ± 14 | 263 ± 44 | 655 ± 126 | 52.8 | 0.031 |

### Example 16. In Vitro Plasma Stability Test for Anti-HER2/TROP2 ADC

H-2B2-T-6F7-ADC, human plasma, monkey plasma and 0.5% BSA PBS solution were each filtered through a 0.22 pum filter for sterilization. H-2B2-T-6F7-ADC was added to the sterile plasma or a solution of 0.5% BSA in PBS at a final concentration of 0.1 mg/mL, and the reaction solution was incubated in an incubator at 37° C.; the day of incubation was noted as day 0, and samples were taken out on day 1, 2, 6, 8, 11 and 14, respectively, for detection of free MMAE by LC-MS (liquid chromatograph-mass spectrometer). The ratio (hereinafter referred to as MMAE release rate (%)) of free MMAE to the total antibody MMAE was calculated.

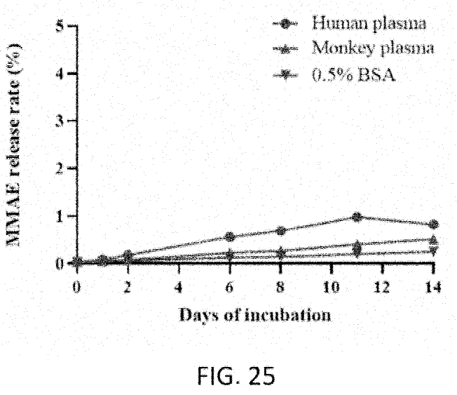

The results were shown in FIG. 25, which indicated that H-2B2-T-6F7-ADC was fairly stable in both human and monkey plasma, as well as a solution of 0.5% BSA in PBS, with a release rate of free MMAE of no more than 1.0% at the highest.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to HER2 (Human epidermal growth factor receptor 2) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 4-6, respectively;
(7) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 4-6, respectively;
(8) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 4-6, respectively;
(9) the selected VH CDRs 1, 2, 3 amino acid sequences are set forth in SEQ ID NOs: 31-33, respectively, and the selected VL CDRs 1, 2, 3 amino acid sequences are set forth in SEQ ID NOs: 4-6, respectively;
(10) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 4-6, respectively.

2. The antibody or antigen-binding fragment thereof of claim 1, wherein the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 1-3, respectively, according to the Kabat numbering scheme.

3. The antibody or antigen-binding fragment thereof of claim 1, wherein the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 1-3, respectively, according to the Kabat numbering scheme.

4. The antibody or antigen-binding fragment thereof of claim 1, wherein the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 1-3, respectively, according to the Kabat numbering scheme.

5. The antibody or antigen-binding fragment thereof of claim 1, wherein the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 1-3, respectively, according to the Kabat numbering scheme.

6. The antibody or antigen-binding fragment thereof of claim 1, wherein the VH comprises CDRs 1, 2, 3 with the amino acid sequences set forth in SEQ ID NOs: 19-21 respectively, and the VL comprises CDRs 1, 2, 3 with the amino acid sequences set forth in SEQ ID NOs: 1-3, respectively, according to the Kabat numbering scheme.

7. The antibody or antigen-binding fragment thereof of claim 1, wherein the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 4-6, respectively, according to the Chothia numbering scheme.

8. The antibody or antigen-binding fragment thereof of claim 1, wherein the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 4-6, respectively, according to the Chothia numbering scheme.

9. The antibody or antigen-binding fragment thereof of claim 1, wherein the VH comprises CDRs 1, 2, 3 with the amino acid sequences set forth in SEQ ID NOs: 28-30 respectively, and the VL comprises CDRs 1, 2, 3 with the amino acid sequences set forth in SEQ ID NOs: 4-6, respectively, according to the Chothia numbering scheme.

10. The antibody or antigen-binding fragment thereof of claim 1, wherein the VH comprises CDRs 1, 2, 3 with the amino acid sequences set forth in SEQ ID NOs: 31-33, respectively, and the VL comprises CDRs 1, 2, 3 with the amino acid sequences set forth in SEQ ID NOs: 4-6, respectively, according to the Chothia numbering scheme.

11. The antibody or antigen-binding fragment thereof of claim 1, wherein the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 4-6, respectively, according to the Chothia numbering scheme.

12. The antibody or antigen-binding fragment thereof of any one of claims 1-11, wherein the antibody or antigen-binding fragment specifically binds to human HER2 or canine HER2.

13. The antibody or antigen-binding fragment thereof of any one of claims 1-12, wherein the antibody or antigen-binding fragment is a human or humanized antibody or antigen-binding fragment thereof.

14. The antibody or antigen-binding fragment thereof of any one of claims 1-13, wherein the antibody or antigen-binding fragment is a single-chain variable fragment (scFV).

15. An antibody or antigen-binding fragment thereof that binds to HER2 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37;
(5) the selected VH sequence is SEQ ID NO:42, and the selected VL sequence is SEQ ID NO: 37.

16. The antibody or antigen-binding fragment thereof of claim 15, wherein the VH comprises the sequence of SEQ ID NO: 38 and the VL comprises the sequence of SEQ ID NO: 37.

17. The antibody or antigen-binding fragment thereof of claim 15, wherein the VH comprises the sequence of SEQ ID NO: 39 and the VL comprises the sequence of SEQ ID NO: 37.

18. The antibody or antigen-binding fragment thereof of claim 15, wherein the VH comprises the sequence of SEQ ID NO: 40 and the VL comprises the sequence of SEQ ID NO: 37.

19. The antibody or antigen-binding fragment thereof of claim 15, wherein the VH comprises the sequence of SEQ ID NO: 41 and the VL comprises the sequence of SEQ ID NO: 37.

20. The antibody or antigen-binding fragment thereof of claim 15, wherein the VH comprises the sequence of SEQ ID NO: 42 and the VL comprises the sequence of SEQ ID NO: 37.

21. The antibody or antigen-binding fragment thereof of any one of claims 15-20, wherein the antibody or antigen-binding fragment specifically binds to human HER2 or canine HER2.

22. The antibody or antigen-binding fragment thereof of any one of claims 15-21, wherein the antibody or antigen-binding fragment is a human or humanized antibody or antigen-binding fragment thereof.

23. The antibody or antigen-binding fragment thereof of any one of claims 15-22, wherein the antibody or antigen-binding fragment is a single-chain variable fragment (scFV).

24. An antibody or antigen-binding fragment thereof that cross-competes with the antibody or antigen-binding fragment thereof of any one of claims 1-23.

25. An antibody or antigen-binding fragment thereof that binds to HER2 comprising
a heavy chain variable region (VH) comprising VH CDR1, VH CDR2, and VH CDR3 that are identical to VH CDR1, VH CDR2, and VH CDR3 of a selected VH sequence; and
a light chain variable region (VL) comprising VL CDR1, VL CDR2, and VL CDR3 that are identical to VL CDR1, VL CDR2, and VL CDR3 of a selected VL sequence,
wherein the selected VH sequence and the selected VL sequence are one of the following:
(1) the selected VH sequence is SEQ ID NO: 38, and the selected VL sequence is SEQ ID NO: 37;
(2) the selected VH sequence is SEQ ID NO: 39, and the selected VL sequence is SEQ ID NO: 37;
(3) the selected VH sequence is SEQ ID NO: 40, and the selected VL sequence is SEQ ID NO: 37;
(4) the selected VH sequence is SEQ ID NO: 41, and the selected VL sequence is SEQ ID NO: 37;
(5) the selected VH sequence is SEQ ID NO: 42, and the selected VL sequence is SEQ ID NO: 37.

26. The antibody or antigen-binding fragment thereof of any one of claims 1-25, wherein the antibody or antigen-binding fragment thereof is a bispecific or a multispecific antibody or an antigen-binding fragment thereof.

27. The antibody or antigen-binding fragment thereof of claim 26, wherein the antibody or antigen-binding fragment thereof further specifically binds to TROP2.

28. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7-9, respectively, and wherein the VH, when paired with a light chain variable region (VL) comprising the amino acid sequence set forth in SEQ ID NO: 37, binds to HER2;
(2) an immunoglobulin heavy chain or a fragment thereof comprising a VH comprising CDRs 1, 2, and 3 comprising the amino acid sequences set forth in SEQ ID NOs: 10-12, respectively, and wherein the VH, when paired with a VL comprising the amino acid sequence set forth in SEQ ID NO: 37, binds to HER2;
(3) an immunoglobulin heavy chain or a fragment thereof comprising a VH comprising CDRs 1, 2, and 3 comprising the amino acid sequences set forth in SEQ ID NOs: 13-15, respectively, and wherein the VH, when paired with a VL comprising the amino acid sequence set forth in SEQ ID NO: 37, binds to HER2;
(4) an immunoglobulin heavy chain or a fragment thereof comprising a VH comprising CDRs 1, 2, and 3 comprising the amino acid sequences set forth in SEQ ID NOs: 16-18, respectively, and wherein the VH, when paired with a VL comprising the amino acid sequence set forth in SEQ ID NO: 37, binds to HER2;
(5) an immunoglobulin heavy chain or a fragment thereof comprising a VH comprising CDRs 1, 2, and 3 comprising the amino acid sequences set forth in SEQ ID NOs: 19-21, respectively, and wherein the VH, when paired with a VL comprising the amino acid sequence set forth in SEQ ID NO: 37 binds to HER2;
(6) an immunoglobulin heavy chain or a fragment thereof comprising a VH comprising CDRs 1, 2, and 3 comprising the amino acid sequences set forth in SEQ ID NOs: 22-24, respectively, and wherein the VH, when paired with a VL comprising the amino acid sequence set forth in SEQ ID NO: 37, binds to HER2;
(7) an immunoglobulin heavy chain or a fragment thereof comprising a VH comprising CDRs 1, 2, and 3 comprising the amino acid sequences set forth in SEQ ID NOs: 25-27, respectively, and wherein the VH, when paired with a VL comprising the amino acid sequence set forth in SEQ ID NO: 37, binds to HER2;
(8) an immunoglobulin heavy chain or a fragment thereof comprising a VH comprising CDRs 1, 2, and 3 comprising the amino acid sequences set forth in SEQ ID NOs: 28-30, respectively, and wherein the VH, when paired with a VL comprising the amino acid sequence set forth in SEQ ID NO: 37, binds to HER2;
(9) an immunoglobulin heavy chain or a fragment thereof comprising a VH comprising CDRs 1, 2, and 3 comprising the amino acid sequences set forth in SEQ ID NOs: 31-33, respectively, and wherein the VH, when paired with a VL comprising the amino acid sequence set forth in SEQ ID NO: 37, binds to HER2;
(10) an immunoglobulin heavy chain or a fragment thereof comprising a VH comprising CDRs 1, 2, and 3 comprising the amino acid sequences set forth in SEQ ID NOs: 34-36, respectively, and wherein the VH, when paired with a VL comprising the amino acid sequence set forth in SEQ ID NO: 37, binds to HER2;
(11) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 38, binds to HER2;
(12) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 39, binds to HER2;
(13) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 40, binds to HER2;
(14) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 41, binds to HER2;
(15) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 42, binds to HER2;
(16) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 38, binds to HER2;
(17) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 39, binds to HER2;
(18) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 40, binds to HER2;
(19) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 41, binds to HER2; or
(20) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 42, binds to HER2.

29. The nucleic acid of claim 28, wherein the VH when paired with a VL specifically binds to human HER2 or canine HER2.

30. The nucleic acid of any one of claims 28-29, wherein the immunoglobulin heavy chain or the fragment thereof is a human or humanized immunoglobulin heavy chain or a fragment thereof.

31. The nucleic acid of any one of claims 28-30, wherein the nucleic acid encodes a single-chain variable fragment (scFv).

32. The nucleic acid of any one of claims 28-31, wherein the nucleic acid is cDNA.

33. An antibody or antigen-binding fragment thereof that binds to TROP2 (Trophoblast cell-surface antigen 2) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 43-45, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 46-48, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 49-51, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 52-54, respectively, and the selected VL CDRs 1, 2, 3 amino acid sequences are set forth in SEQ ID NOs: 4-6, respectively;
(5) the selected VH CDRs 1, 2, 3 amino acid sequences are set forth in SEQ ID NOs: 55-57, respectively, and the selected VL CDRs 1, 2, 3 amino acid sequences are set forth in SEQ ID NOs: 4-6, respectively;
(6) the selected VH CDRs 1, 2, 3 amino acid sequences are set forth in SEQ ID NOs: 58-60, respectively, and the selected VL CDRs 1, 2, 3 amino acid sequences are set forth in SEQ ID NOs: 4-6, respectively.

34. The antibody or antigen-binding fragment thereof of claim 33, wherein the VH comprises CDRs 1, 2, 3 with the amino acid sequences set forth in SEQ ID NOs: 43-45, respectively, and the VL comprises CDRs 1, 2, 3 with the amino acid sequences set forth in SEQ ID NOs: 1-3, respectively, according to the Kabat numbering scheme.

35. The antibody or antigen-binding fragment thereof of claim 33, wherein the VH comprises CDRs 1, 2, 3 with the amino acid sequences set forth in SEQ ID NOs: 46-48, respectively, and the VL comprises CDRs 1, 2, 3 with the amino acid sequences set forth in SEQ ID NOs: 1-3, respectively, according to the Kabat numbering scheme.

36. The antibody or antigen-binding fragment thereof of claim 33, wherein the VH comprises CDRs 1, 2, 3 with the amino acid sequences set forth in SEQ ID NOs: 49-51, respectively, and the VL comprises CDRs 1, 2, 3 with the amino acid sequences set forth in SEQ ID NOs: 1-3, respectively, according to the Kabat numbering scheme.

37. The antibody or antigen-binding fragment thereof of claim 33, wherein the VH comprises CDRs 1, 2, 3 with the amino acid sequences set forth in SEQ ID NOs: 52-54, respectively, and the VL comprises CDRs 1, 2, 3 with the amino acid sequences set forth in SEQ ID NOs: 4-6, respectively, according to the Chothia numbering scheme.

38. The antibody or antigen-binding fragment thereof of claim 33, wherein the VH comprises CDRs 1, 2, 3 with the amino acid sequences set forth in SEQ ID NOs: 55-57, respectively, and the VL comprises CDRs 1, 2, 3 with the amino acid sequences set forth in SEQ ID NOs: 4-6, respectively, according to the Chothia numbering scheme.

39. The antibody or antigen-binding fragment thereof of claim 33, wherein the VH comprises CDRs 1, 2, 3 with the amino acid sequences set forth in SEQ ID NOs: 58-60 respectively, and the VL comprises CDRs 1, 2, 3 with the amino acid sequences set forth in SEQ ID NOs: 4-6, respectively, according to the Chothia numbering scheme.

40. The antibody or antigen-binding fragment thereof of any one of claims 33-39, wherein the antibody or antigen-binding fragment specifically binds to human TROP2 or canine TROP2.

41. The antibody or antigen-binding fragment thereof of any one of claims 33-40, wherein the antibody or antigen-binding fragment is a human or humanized antibody or antigen-binding fragment thereof.

42. The antibody or antigen-binding fragment thereof of any one of claims 33-41, wherein the antibody or antigen-binding fragment is a single-chain variable fragment (scFV).

43. An antibody or antigen-binding fragment thereof that binds to TROP2 comprising a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 61, and the selected VL sequence is SEQ ID NO: 37;
(2) the selected VH sequence is SEQ ID NO: 62, and the selected VL sequence is SEQ ID NO: 37;
(3) the selected VH sequence is SEQ ID NO: 63, and the selected VL sequence is SEQ ID NO: 37.

44. The antibody or antigen-binding fragment thereof of claim 43, wherein the VH comprises the sequence of SEQ ID NO: 61 and the VL comprises the sequence of SEQ ID NO: 37.

45. The antibody or antigen-binding fragment thereof of claim 43, wherein the VH comprises the sequence of SEQ ID NO: 62 and the VL comprises the sequence of SEQ ID NO: 37.

46. The antibody or antigen-binding fragment thereof of claim 43, wherein the VH comprises the sequence of SEQ ID NO: 63 and the VL comprises the sequence of SEQ ID NO: 37.

47. The antibody or antigen-binding fragment thereof of any one of claims 43-46, wherein the antibody or antigen-binding fragment specifically binds to human TROP2 or canine TROP2.

48. The antibody or antigen-binding fragment thereof of any one of claims 43-47, wherein the antibody or antigen-binding fragment is a human or humanized antibody or antigen-binding fragment thereof.

49. The antibody or antigen-binding fragment thereof of any one of claims 43-46, wherein the antibody or antigen-binding fragment is a single-chain variable fragment (scFV).

50. An antibody or antigen-binding fragment thereof that cross-competes with the antibody or antigen-binding fragment thereof of any one of claims 33-49.

51. An antibody or antigen-binding fragment thereof that binds to TROP2 comprising a heavy chain variable region (VH) comprising VH CDR1, VH CDR2, and VH CDR3 that are identical to VH CDR1, VH CDR2, and VH CDR3 of a selected VH sequence; and
a light chain variable region (VL) comprising VL CDR1, VL CDR2, and VL CDR3 that are identical to VL CDR1, VL CDR2, and VL CDR3 of a selected VL sequence,
wherein the selected VH sequence and the selected VL sequence are one of the following:
(1) the selected VH sequence is SEQ ID NO: 61, and the selected VL sequence is SEQ ID NO: 37;
(2) the selected VH sequence is SEQ ID NO: 62, and the selected VL sequence is SEQ ID NO: 37;
(3) the selected VH sequence is SEQ ID NO: 63, and the selected VL sequence is SEQ ID NO: 37.

52. The antibody or antigen-binding fragment thereof of any one of claims 33-51, wherein the antibody or antigen-binding fragment thereof is a bispecific or multispecific antibody or an antigen-binding fragment thereof.

53. The antibody or antigen-binding fragment thereof of any one of claims 33-52, wherein the antibody or antigen-binding fragment thereof further specifically binds to HER2.

54. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 43-45, respectively, and wherein the VH, when paired with a light chain variable region (VL) comprising the amino acid sequence set forth in SEQ ID NO: 37, binds to TROP2;
(2) an immunoglobulin heavy chain or a fragment thereof comprising a VH comprising CDRs 1, 2, and 3 comprising the amino acid sequences set forth in SEQ ID NOs: 46-48, respectively, and wherein the VH, when paired with a VL comprising the amino acid sequence set forth in SEQ ID NO: 37, binds to TROP2;
(3) an immunoglobulin heavy chain or a fragment thereof comprising a VH comprising CDRs 1, 2, and 3 comprising the amino acid sequences set forth in SEQ ID NOs: 49-51, respectively, and wherein the VH, when paired with a VL comprising the amino acid sequence set forth in SEQ ID NO: 37, binds to TROP2; or
(4) an immunoglobulin heavy chain or a fragment thereof comprising a VH comprising CDRs 1, 2, and 3 comprising the amino acid sequences set forth in SEQ ID NOs: 52-54, respectively, and wherein the VH, when paired with a VL comprising the amino acid sequence set forth in SEQ ID NO: 37, binds to TROP2;
(5) an immunoglobulin heavy chain or a fragment thereof comprising a VH comprising CDRs 1, 2, and 3 comprising the amino acid sequences set forth in SEQ ID NOs: 55-57, respectively, and wherein the VH, when paired with a VL comprising the amino acid sequence set forth in SEQ ID NO: 37, binds to TROP2;
(6) an immunoglobulin heavy chain or a fragment thereof comprising a VH comprising CDRs 1, 2, and 3 comprising the amino acid sequences set forth in SEQ ID NOs: 58-60, respectively, and wherein the VH, when paired with a VL comprising the amino acid sequence set forth in SEQ ID NO: 37, binds to TROP2;
(7) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 61, binds to TROP2;
(8) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 62, binds to TROP2;
(9) an immunoglobulin light chain or a fragment thereof comprising a light chain variable region (VL) comprising complementarity determining regions (CDRs) 1, 2, and 3 comprising the amino acid sequences set forth in SEQ ID NOs: 1-3, respectively, and wherein the VL, when paired with a heavy chain variable region (VH) comprising the amino acid sequence set forth in SEQ ID NO: 63, binds to TROP2;
(10) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 61, binds to TROP2;
(11) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 62, binds to TROP2;
(12) an immunoglobulin light chain or a fragment thereof comprising a light chain variable region (VL) comprising complementarity determining regions (CDRs) 1, 2, and 3 comprising the amino acid sequences set forth in SEQ ID NOs: 4-6, respectively, and wherein the VL, when paired with a heavy chain variable region (VH) comprising the amino acid sequence set forth in SEQ ID NO: 63, binds to TROP2.

55. The nucleic acid of claim 54, wherein the VH when paired with a VL specifically binds to human TROP2 or canine TROP2.

56. The nucleic acid of any one of claims 54-55, wherein the immunoglobulin heavy chain or the fragment thereof is a human or humanized immunoglobulin heavy chain or a fragment thereof.

57. The nucleic acid of any one of claims 54-56, wherein the nucleic acid encodes a single-chain variable fragment (scFv).

58. The nucleic acid of any one of claims 54-57, wherein the nucleic acid is cDNA.

59. An antigen-binding protein construct, comprising: a first antigen-binding domain that specifically binds to HER2; and a second antigen-binding domain that specifically binds to TROP2.

60. The antigen-binding protein construct of claim 59, wherein the first antigen-binding domain comprises a first heavy chain variable region (VH1) and a first light chain variable region (VL1); and the second antigen-binding domain comprises a second heavy chain variable region (VH2) and a second light chain variable region (VL2).

61. The antigen-binding protein construct of claim 60, wherein
the first heavy chain variable region (VH1) comprising complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprising CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, the selected VL1 CDRs 1, 2, and 3 amino acid sequences are one of the following:
(1) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 22-24, respectively, and the selected VL1 CDRs 1, 2, 3 amino acid sequences are set forth in SEQ ID NOs: 4-6, respectively;
(7) the selected VH1 CDRs 1, 2, 3 amino acid sequences are set forth in SEQ ID NOs: 25-27, respectively, and the selected VL1 CDRs 1, 2, 3 amino acid sequences are set forth in SEQ ID NOs: 4-6, respectively;
(8) the selected VH1 CDRs 1, 2, 3 amino acid sequences are set forth in SEQ ID NOs: 28-30, respectively, and the selected VL1 CDRs 1, 2, 3 amino acid sequences are set forth in SEQ ID NOs: 4-6, respectively;
(9) the selected VH1 CDRs 1, 2, 3 amino acid sequences are set forth in SEQ ID NOs: 31-33, respectively, and the selected VL1 CDRs 1, 2, 3 amino acid sequences are set forth in SEQ ID NOs: 4-6, respectively;
(10) the selected VH1 CDRs 1, 2, 3 amino acid sequences are set forth in SEQ ID NOs: 34-36, respectively, and the selected VL1 CDRs 1, 2, 3 amino acid sequences are set forth in SEQ ID NOs: 4-6, respectively.

62. The antigen-binding protein construct of claim 60 or claim 61, wherein
the second heavy chain variable region (VH2) comprising CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprising CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(2) the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(3) the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(4) the selected VH2 CDRs 1, 2, 3 amino acid sequences are set forth in SEQ ID NOs: 52-54, respectively, and the selected VL2 CDRs 1, 2, 3 amino acid sequences are set forth in SEQ ID NOs: 4-6, respectively;
(5) the selected VH2 CDRs 1, 2, 3 amino acid sequences are set forth in SEQ ID NOs: 55-57, respectively, and the selected VL2 CDRs 1, 2, 3 amino acid sequences are set forth in SEQ ID NOs: 4-6, respectively;
(6) the selected VH2 CDRs 1, 2, 3 amino acid sequences are set forth in SEQ ID NOs: 58-60, respectively, and the selected VL2 CDRs 1, 2, 3 amino acid sequences are set forth in SEQ ID NOs: 4-6, respectively.

63. The antigen-binding protein construct of any one of claims 60-62, wherein
(1) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(2) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(3) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(4) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(5) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(6) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(7) the selected VH1 CDRs 1, 2, 3 amino acid sequences are set forth in SEQ ID NOs: 7-9, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(8) the selected VH1 CDRs 1, 2, 3 amino acid sequences are set forth in SEQ ID NOs: 10-12, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(9) the selected VH1 CDRs 1, 2, 3 amino acid sequences are set forth in SEQ ID NOs: 19-21, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(10) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(11) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(12) the selected VH1 CDRs 1, 2, 3 amino acid sequences are set forth in SEQ ID NOs: 13-15, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively;
(13) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 49-51, respectively, and the selected VL2 CDRs 1, 2, 3 amino acid sequences are set forth in SEQ ID NOs: 1-3, respectively;
(14) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 46-48, respectively, and the selected VL2 CDRs 1, 2, 3 amino acid sequences are set forth in SEQ ID NOs: 1-3, respectively;
(15) the selected VH1 CDRs 1, 2, 3 amino acid sequences are set forth in SEQ ID NOs: 16-18, respectively, and the selected VL1 CDRs 1, 2, 3 amino acid sequences are set forth in SEQ ID NOs: 1-3, respectively, and the selected VH2 CDRs 1, 2, 3 amino acid sequences are set forth in SEQ ID NOs: 43-45, respectively, and the selected VL2 CDRs 1, 2, 3 amino acid sequences are set forth in SEQ ID NOs: 1-3, respectively.

64. The antigen-binding protein construct of claim 60, wherein the first heavy chain variable region comprises a sequence that is at least 80%, 85%, 90%, or 95% identical to SEQ ID NO: 39, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 63, and the second light chain variable region comprises a sequence that is at least 80%, 85%, 90%, or 95% identical to SEQ ID NO: 37.

65. The antigen-binding protein construct of claim 60, wherein the first heavy chain variable region comprises a sequence that is at least 80%, 85%, 90%, or 95% identical to SEQ ID NO: 42, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 63, and the second light chain variable region comprises a sequence that is at least 80%, 85%, 90%, or 95% identical to SEQ ID NO: 37.

66. The antigen-binding protein construct of claim 60, wherein the first heavy chain variable region comprises a sequence that is at least 80%, 85%, 90%, or 95% identical to SEQ ID NO: 38, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 63, and the second light chain variable region comprises a sequence that is at least 80%, 85%, 90%, or 95% identical to SEQ ID NO: 37.

67. The antigen-binding protein construct of claim 60, wherein the first heavy chain variable region comprises a sequence that is at least 80%, 85%, 90%, or 95% identical to SEQ ID NO: 38, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 62, and the second light chain variable region comprises a sequence that is at least 80%, 85%, 90%, or 95% identical to SEQ ID NO: 37.

68. The antigen-binding protein construct of claim 60, wherein the first heavy chain variable region comprises a sequence that is at least 80%, 85%, 90%, or 95% identical to SEQ ID NO: 39, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 62, and the second light chain variable region comprises a sequence that is at least 80%, 85%, 90%, or 95% identical to SEQ ID NO: 37.

69. The antigen-binding protein construct of claim 60, wherein the first heavy chain variable region comprises a sequence that is at least 80%, 85%, 90%, or 95% identical to SEQ ID NO: 42, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 62, and the second light chain variable region comprises a sequence that is at least 80%, 85%, 90%, or 95% identical to SEQ ID NO: 37.

70. The antigen-binding protein construct of claim 60, wherein the first heavy chain variable region comprises a sequence that is at least 80%, 85%, 90%, or 95% identical to SEQ ID NO: 38, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 61, and the second light chain variable region comprises a sequence that is at least 80%, 85%, 90%, or 95% identical to SEQ ID NO: 37.

71. The antigen-binding protein construct of claim 60, wherein the first heavy chain variable region comprises a sequence that is at least 80%, 85%, 90%, or 95% identical to SEQ ID NO: 39, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 61, and the second light chain variable region comprises a sequence that is at least 80%, 85%, 90%, or 95% identical to SEQ ID NO: 37.

72. The antigen-binding protein construct of claim 60, wherein the first heavy chain variable region comprises a sequence that is at least 80%, 85%, 90%, or 95% identical to SEQ ID NO: 42, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 61, and the second light chain variable region comprises a sequence that is at least 80%, 85%, 90%, or 95% identical to SEQ ID NO: 37.

73. The antigen-binding protein construct of claim 60, wherein the first heavy chain variable region comprises a sequence that is at least 80%, 85%, 90%, or 95% identical to SEQ ID NO: 40, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 63, and the second light chain variable region comprises a sequence that is at least 80%, 85%, 90%, or 95% identical to SEQ ID NO: 37.

74. The antigen-binding protein construct of claim 60, wherein the first heavy chain variable region comprises a sequence that is at least 80%, 85%, 90%, or 95% identical to SEQ ID NO: 41, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 63, and the second light chain variable region comprises a sequence that is at least 80%, 85%, 90%, or 95% identical to SEQ ID NO: 37.

75. The antigen-binding protein construct of claim 60, wherein the first heavy chain variable region comprises a sequence that is at least 80%, 85%, 90%, or 95% identical to SEQ ID NO: 40, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 62, and the second light chain variable region comprises a sequence that is at least 80%, 85%, 90%, or 95% identical to SEQ ID NO: 37.

76. The antigen-binding protein construct of claim 60, wherein the first heavy chain variable region comprises a sequence that is at least 80%, 85%, 90%, or 95% identical to SEQ ID NO: 41, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 62, and the second light chain variable region comprises a sequence that is at least 80%, 85%, 90%, or 95% identical to SEQ ID NO: 37.

77. The antigen-binding protein construct of claim 60, wherein the first heavy chain variable region comprises a sequence that is at least 80%, 85%, 90%, or 95% identical to SEQ ID NO: 40, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 61, and the second light chain variable region comprises a sequence that is at least 80%, 85%, 90%, or 95% identical to SEQ ID NO: 37.

78. The antigen-binding protein construct of claim 60, wherein the first heavy chain variable region comprises a sequence that is at least 80%, 85%, 90%, or 95% identical to SEQ ID NO: 41, the first light chain variable region comprises a sequence that is at least 80%, 85%, 90%, or 95% identical to SEQ ID NO: 37, the second heavy chain variable region comprises a sequence that is at least 80%, 85%, 90%, or 95% identical to SEQ ID NO: 61, and the second light chain variable region comprises a sequence that is at least 80%, 85%, 90%, or 95% identical to SEQ ID NO: 37.

79. The antigen-binding protein construct of any one of claims 59-78, wherein the antigen-binding protein construct is a bispecific antibody.

80. The antigen-binding protein construct of any one of claims 60-78, wherein the first light chain variable region and the second light chain variable region are identical.

81. A vector comprising one or more of the nucleic acids of any one of claims 28-32 and 54-58, or a nucleic acid encoding the antibody or antigen-binding fragment thereof of any one of claims 1-27 and 33-53, or a nucleic acid encoding the antigen-binding protein construct of any one of claims 59-80.

82. A cell comprising the vector of claim 81.

83. The cell of claim 82, wherein the cell is a CHO cell.

84. A cell comprising one or more of the nucleic acids of any one of claims 28-32 and 54-58, or a nucleic acid encoding the antibody or antigen-binding fragment thereof of any one of claims 1-27 and 33-53, or a nucleic acid encoding the antigen-binding protein construct of any one of claims 59-80.

85. A method of producing an antibody or an antigen-binding fragment thereof, or an antigen-binding protein construct, the method comprising
(a) culturing the cell of any one of claims 82-84 under conditions sufficient for the cell to produce the antibody or the antigen-binding fragment thereof, or the antigen-binding protein construct; and
(b) collecting the antibody or the antigen-binding fragment thereof, or the antigen-binding protein construct produced by the cell.

86. An antibody-drug conjugate comprising a therapeutic agent covalently bound to:
(a) the antibody or antigen-binding fragment thereof of any one of claims 1-27 and 33-53; or
(b) the antigen-binding protein construct of any one of claims 59-80.

87. The antibody drug conjugate of claim 86, wherein the therapeutic agent is a cytotoxic or cytostatic agent.

88. The antibody drug conjugate of claim 86 or claim 87, wherein the therapeutic agent is MMAE or MMAF.

89. A method of treating a subject having cancer, the method comprising administering a therapeutically effective amount of a composition comprising the antibody or antigen-binding fragment thereof of any one of claims 1-27 and 33-53, the antigen-binding protein construct of any one of claims 59-80, or the antibody-drug conjugate of any one of claims 86-88, to the subject.

90. The method of claim 89, wherein the subject has a solid tumor.

91. The method of claim 89, wherein the cancer is thyroid cancer, urothelial cancer, breast cancer, colorectal cancer, renal cancer, cervical cancer, ovarian cancer, lung cancer, endometrial cancer, skin cancer, stomach cancer, esophageal carcinoma, pancreatic cancer, prostate cancer, liver cancer, lymphoma, or glioma.

92. The method of claim 89, wherein the cancer is cervical cancer, prostate cancer, thyroid cancer, urothelial cancer, head and neck cancer, endometrial cancer, ovarian cancer, lung cancer, breast cancer, carcinoid, skin cancer, liver cancer, or testis cancer.

93. The method of claim 89, wherein the cancer is multiple myeloma or renal carcinoma.

94. The method of any one of claims 89-93, wherein the subject is a human.

95. The method of any one of claims 89-93, wherein the subject is a non-human animal.

96. A method of decreasing the rate of tumor growth, the method comprising contacting a tumor cell with an effective amount of a composition comprising the antibody or antigen-binding fragment thereof of any one of claims 1-27 and 33-53, the antigen-binding protein construct of any one of claims 59-80, or the antibody-drug conjugate of any one of claims 86-88.

97. A method of killing a tumor cell, the method comprising contacting a tumor cell with an effective amount of a composition comprising the antibody or antigen-binding fragment thereof of any one of claims 1-27 and 33-53, the antigen-binding protein construct of any one of claims 59-80, or the antibody-drug conjugate of any one of claims 86-88.

98. A pharmaceutical composition comprising a pharmaceutically acceptable carrier and
(a) the antibody or antigen-binding fragment thereof of any one of claims 1-27 and 33-53,
(b) the antigen-binding protein construct of any one of claims 59-80, or
(c) the antibody-drug conjugate of any one of claims 86-88.
